(19) United States
(12) Patent Application Publication
(10) Pub. No.: US 2005/0229260 A1
(43) Pub. Date: Oct. 13, 2005

(54) Compound screens relating to insulin deficiency or insulin resistance

(75) Inventors: Richard Feichtinger, Gent (BE); Thierry Bogaert, Kortrijk (BE)

Correspondence Address: WOLF GREENFIELD & SACKS, PC;FEDERAL RESERVE PLAZA, 600 ATLANTIC AVENUE, BOSTON, MA, 02210-2211, US

(73) Assignee: DevGen NV, Zwijnaarde (BE)

(21) Appl. No.: 10/297,336

(22) PCT Filed: Jun. 8, 2001
(86) PCT No.: PCT/IB01/01199

§ 371 Date: Jul. 18, 2003

(30) Foreign Application Priority Data
Jun. 8, 2000 (GB) 0014009.5

(51) Int. Cl. A01K 67/033

(52) U.S. Cl. 800/3; 800/8

(57) ABSTRACT

The invention is concerned with use of the model organism C. elegans as a research tool to screen for compounds active in insulin signalling. In particular, the invention relates to improved screening methods based on release of C. elegans from the dauer larval state.

The present invention is concerned with using the model organism C. elegans as a research tool to effectively screen compound libraries for compounds active in insulin signalling, in particular compounds which act downstream of the insulin receptor. Specifically the invention relates to improved screening methods based on release of C. elegans from the dauer larval state.

In a particular embodiment, the invention provides improved screening methods using C. elegans carrying mutations in one or more gene(s) involved in the insulin signalling pathway, such as the Daf-genes. In one particular embodiment, (at least one of) said mutation(s) is in the daf-2 gene, which is homologous to the insulin receptor subfamily of receptor tyrosine kinases. One the basis of the homology between daf-2 and the insulin receptor subfamily it is proposed that worms mutant in the daf-2 gene may serve as models for insulin-related diseases and disease risks, as for example diabetes mellitus, obesity, insulin resistance and impaired glucose tolerance (Kimura et al. 1997, Science 277, 942-946).

General techniques and methodology for performing in vivo assays using the nematode worm Caenorhabditis elegans (C. elegans) as a model organism have been described in the art, most notably in the following applications by applicant: PCT/EP99/09710 (published on 15 Jun. 2000 as WO 00/34438); PCT/EP99/04718 (published on Jan. 15, 2000 as WO/00/01846); PCT/IB00/00575 (published on Oct. 26, 2000 as WO 00/63427); PCT/IB00/00557 (published on Oct. 26, 2000 as WO 00/63425); PCT/IB00/00558 (published on Oct. 26, 2000 as WO 00/63426); as well as for instance PCT/US98/10080 (published on Nov. 19, 1998 as WO 98/51351), PCT/US99/13650, PCT/US99/01361 (published on 29-07-1999 as WO99/37770), and PCT/EP00/05102.

As described in these applications, one of the main advantages of assays involving the use of C. elegans is that such assays can be carried out in multi-well plate format (with each well usually containing a sample of between 2 and 100 worms) and—also because of this—may also be carried out in an automated fashion, i.e. using suitable robotics (as are described in the aforementioned applications and/or as may be commercially available). This makes assays involving the use of C. elegans ideally suited for screening of libraries of chemical compounds, in particular at medium to high throughput. Such automated screens may for instance be used in the discovery and/or development of new compounds (e.g. small molecules) for pharmaceutical, veterinary or agrochemical/pesticidal (e.g. insecticidal and/or nematocidal) use.

Some other advantages associated with the use of C. elegans as a model organism (e.g. in the assay techniques referred to above) include, but are not limited to:
C. elegans has a short life-cycle of about 3 days. This not only means that these nematodes (and suitable mutants, transgenics and/or stable lines thereof) can be cultivated/generated quickly and in high numbers, but also allows assays using C. elegans to test, in a relatively short period of time and at high throughput, the nematode worms over one or more, and up to all, stages of life/development, and even over one or more generations. Also, because of this short life span, in C. elegans based-assays, compounds may be tested over one or more, and up to essentially all, stages of development, without any problems associated with compound stability and/or (bio)availability;
C. elegans is transparent, allowing—with advantage—for visual or non-visual inspection of internal organs and internal processes, and also the use of markers such as fluorescent reporter proteins, even while the worms are still alive. Also, as further mentioned below, such inspection may be carried out in automated fashion using suitable equipment such as plate readers;
C. elegans is a well-established and well-characterized model organism. For example, the genome of C. elegans has been fully sequenced, and also the complete lineage and cell interactions (for example of synapses) are known. In addition, C. elegans has full diploid genetics, and is capable of both sexual reproduction (e.g. for crossing) as well as reproduction as a self-fertilizing hermaphrodite. All this may provide many advantages, not only for the use of C. elegans in genetic and/or biological studies, but also for the use of C. elegans in the discovery, development and/or pharmacology of (candidate) drugs for human or animal use.

Techniques for transforming, handling, cultivating, maintaining and storing (e.g. as frozen samples, which offers great practical advantages) C. elegans are well established in the art, for instance from the handbooks referred to below. For example, C. elegans may be used as one or more samples with essentially fully isogenic genotype(s).

Generally, in the assays described above, the nematodes are incubated in suitable vessel or container—such as a compartment or well of a multi-well plate—on a suitable medium (which may be a solid, semi-solid, viscous or liquid medium, with liquid and viscous media usually being preferred for assays in multi-well plate format). The nematodes are then contacted with the compound(s) to be tested, e.g. by adding the compound to the medium containing the worms. After a suitable incubation time (i.e. sufficient for the compound to have its effect—if any—on the nematodes), the worms are then subjected to a suitable detection technique, i.e. to measure/determine a signal that is representative for the influence of the compound(s) to be tested on the nematode worms, which may then be used as a measure for the activity of the compound(s) in the in vivo assay.

Often, in particular for automated assays, such a detection technique involves a non-visual detection method (as further described in the applications mentioned above), such as measurement of fluorescence or another optical method, measurement of a particular marker (either associated with worms or associated with the medium) such as autonomous fluorescent proteins (AFP's) such as green fluorescent proteins (GFP's), aequorin, alkaline phosphatase, luciferase, Beta-glucoronidase, Beta-lactamase, Beta-galactosidase, acetohydroxyacid, chloramphenicol acetyl transferase, horse radish peroxidase, nopaline synthase, or octapine synthase. For example, for automated assays carried out in multi-well plates, so called (multi-well) “plate readers” may be used for detecting/measuring said signal.

For a further description of the above and other assay techniques involving the use of nematodes as a model organism, reference is made to the prior art, such as the applications by applicant referred to above.

For general information on C. elegans and techniques for handling this nematode worm, reference is made to the standard handbooks, such as W. B. Wood et al., “The nematode Caenorhabditis elegans”, Cold Spring Harbor Laboratory Press (1988) and D. L. Riddle et al., “C. ELEGANS II”, Cold Spring Harbor Laboratory Press (1997).

The use of C. elegans based assays in the field of metabolic diseases—such as obesity and diabetes—has been described in a number of applications, most notably in PCT US 98/10800 and U.S. Pat. No. 6,225,120, which relate to the use of daf-2 mutant C. elegans nematodes for selecting compounds active in impaired glucose tolerance and diabetes, as a model for insulin resistance.

One of the main objects of the present invention is to provide improved methods for the selection of compounds for the field of metabolic diseases—including but not limited to obesity, impaired glucose tolerance and type-II diabetes—which methods may be used for drug discovery, development, pharmacology and testing. In particular, it is an object of the invention to provide such improved assays as compared to the assay techniques described in PCT US 98/10800 and U.S. Pat. No. 6,225,120.

Generally, the invention solves this problem by the use, in such assays, of nematode strains (such as m41) which have increased sensitivity of the insulin signalling pathway compared to the strains used in PCT US 98/10800 and U.S. Pat. No. 6,225,120.

Diabetes mellitus is a major growing public health problem in both developed and developing countries. Including clinical complications it accounts for 5% of the total healthcare expenditure in Europe. Depending on the type of diabetes, current drug therapy strategy for diabetes consist of a diet supported by either application of exogenous insulin of different origin, application of drugs that increase production and/or release of endogenous insulin, enhance sensitivity of peripheral organs to insulin or mimic insulin effects. Drugs acting directly in the insulin pathway downstream of the receptor are potentially beneficial in both major types of diabetes but they are not existing today. The major drawback of currently available drugs is the body weight gain that comes on top of an existing obesity in the vast majority (80%) of patients. This side effect is also the main reason why pharmacological intervention in the middle range of disease development is not as intense and aggressive, as it should be to achieve optimal efficacy. New drugs that are devoid of this side effect would already reduce risk of complications by 12 to 30% (United Kingdom prospective diabetes study. Turner et al. 1998, BMJ 316: 823-828; Turner et al. 1999, JAMA 281: 2005-2012).

Novel glitazones, such as troglitazone, that act on nuclear receptors which regulate carbohydrate metabolism that have been launched in Japan and the US were withdrawn due to an elevated risk of liver toxicity. Hence the medical need for well tolerated orally-active anti-diabetics with mild benign side-effects remains high. A compound that directly interacts downstream the insulin receptor pathway could establish a breakthrough especially since it could be a drug that acts both in Type I and Type II diabetes. A compound that has as a clinical result an insulin sparing effect could also be of extremely high therapeutic value.

From animal studies inorganic vanadates are known to favourably combine increase in insulin sensitivity and reduction of hyperlipidemia together with body weight stability or loss, but are devoid of body weight gain (Brichard and Henquin 1995, TiPS 16: 265-270). Due to unresolved toxicity issues, however, they are not available in drug formulas. Although inorganic vanadium compounds are currently in clinical trial, the issue of side effects still raises doubts for this class of compounds to have to specification of a drug, which has to be well tolerated in multiple doses per day for decades.

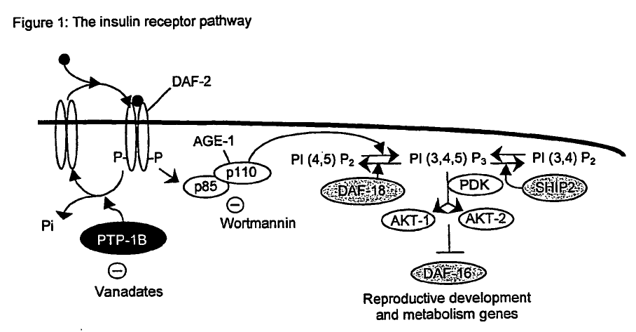

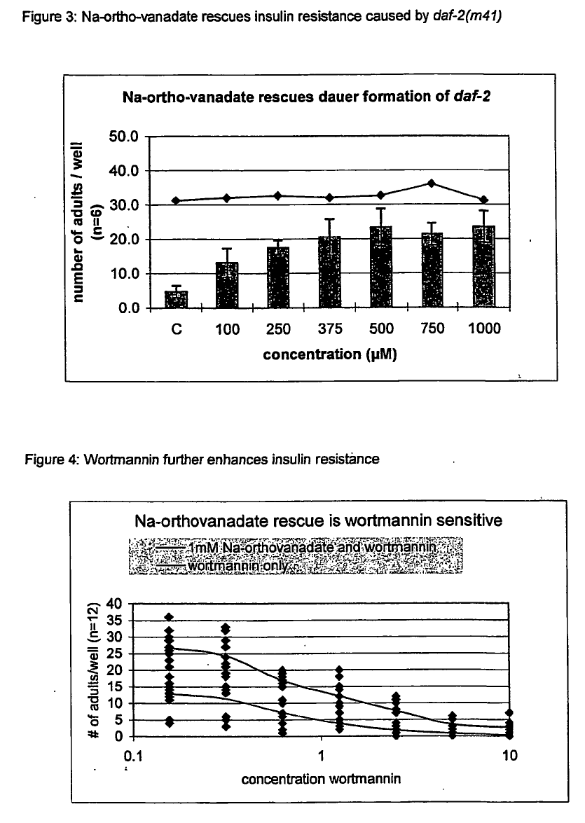

Nevertheless, the recognition of protein tyrosine phosphatase 1B as the major target of vanadates and the validation of this target as strongly increasing insulin sensitivity when inactivated in mice points towards the insulin receptor pathway as valuable for finding active compounds to ameliorate insulin resistance (Elchebly et al. 1999, Science 283: 1544-1548). PTP-1B is a negative regulator of insulin receptor tyrosine phosphorylation and kinase activity, its inactivation is raising insulin signalling with given constant insulin levels (FIG. 1). The present inventors have shown that vanadates can rescue the genetic insulin resistance caused by daf-2 mutations in Caenorhabditis elegans, thereby validating the genetic model for insulin-deficient and insulin-resistant related disease by pharmacological means (FIG. 3). Wortmannin, an inhibitor of the downstream effector phosphatidyl-inositol-3-phosphat kinase (FIG. 1), further increases insulin resistance, confirming the sensitivity of the invented assay for the pathway (FIG. 4). The possible known targets in the insulin-receptor pathway shown in FIG. 1 are listed in table 1.

The inventors have made two key adaptations which enable them to use C. elegans mutant strains to effectively screen large compound libraries for activities mimicking vanadates using screens based on rescue of the phenotype dauer formation and other phenotypic traits which are caused by interventions in the insulin signalling pathway, such as, for example, mutations in the insulin receptor gene homologue daf-2. The first adaptation is the use of C. elegans with a sensitized genetic background; the second adaptation is manipulation of the assay conditions such that a basal level of release from the dauer larval state is present even in the absence of test compounds. The daf-2 gene had previously been disregarded as useful target for compound screens due to a failure of obtaining active compounds from large compound libraries (Carl Johnson, Axys pharmaceuticals, Nemapharm division, disclosed at the Cold Spring Harbor worm course). The new developments described herein overcome sensitivity problems previously encountered with screens based on daf-2.

In the invention, generally nematode strains are used that show sensitivity of the insulin signalling pathway.

In particular, these strains are used in assays involving the use of a dauer stage and/or dauer phenotype as a read out. These may for instance be assays based on “dauer rescue” and/or on “dauer formation/bypass” (of which dauer bypass is usually preferred, as it may avoid the problems associated with the limited uptake of the compound(s) to be tested by worms in the dauer state).

In the former type of assay, a sample of worms in the dauer state is provided, and the efficacy of the compound(s) to be tested in bringing the worms of said sample out of the dauer state is determined. Generally, compounds with the desired activity will bring the worms out of the dauer state (i.e. to a greater degree than a reference without compound, and preferably in a dose/concentration-dependant manner) and thus provide adults (i.e. more adults than without the presence of the compound(s) to be tested).

In the latter type of assay, a sample of worms (in particular eggs, L1 or l2 worms, and preferably L1 worms) is kept under conditions which, without the presence of any compound(s) to be tested, would cause (most and preferably essentially all) of the worms in the sample to enter the dauer state, and the efficacy of the compound(s) to be tested in preventing the worms, under these conditions, to enter the dauer state (i.e. to bypass the dauer state) is determined. Generally, compounds with the desired activity will prevent the worms from entering the dauer state (i.e. to a greater degree than a reference without compound, and preferably in a dose/concentration-dependant manner) and thus provide adults (i.e. more adults than without the presence of the compound(s) to be tested, and preferably in a dose-dependant manner). Conditions such that the worm strain(s) used will enter the dauer state without the presence of the compound(s) to be tested will depend on the specific worms strain used and will be clear to the skilled person, also in view of the preferred conditions described hereinbelow. Also, these conditions are preferably such that, under the conditions of the assay, a reference compound with the desired activity (such as vanadate at a concentration of between 0.5 and 2 milliMolar) will allow a measurable amount of worms to bypass the dauer state (e.g. between 40 to 70%, or even more). If necessary, the results obtained with such a reference compound may also serve as a positive control or comparative reference for the compound(s) to be tested.

As will be clear to the skilled person, for both the dauer rescue and the dauer bypass assays described above, and during or at the end of the assay, either the number of dauer larvae in the sample and/or the number of adults may be determined (with the sum of the number of dauer larvae and the number of adults being essentially equal to the number of worms present in the original sample). Techniques for determining the number of adults and/or dauer larvae in a sample will be clear to the skilled person and may include visual inspection of the sample (e.g. counting) as well as the automated non-visual detection techniques referred to above.

In the context of the present invention, the insulin signalling pathway may generally be described in all enzymatic conversions and other signal transduction events that are involved in (transmembrane) receptor-mediated (cellular) signal transduction in response to the (extracellular) presence insulin signals (e.g. the extracellular presence of insulin or insulin-like compounds). Some of the most important (but non-limiting) examples of the different enzymatic conversions involved in said signalling have already been mentioned hereinabove.

By “sensitivity of the insulin signalling pathway” is generally meant that
1) the nematode shows one or more biological response(s) to the presence of an insulin, to the presence of an insulin-like compound, and/or to the presence of a compound that can provide and/or or mimic a biological response similar to the biological response(s) provided by insulin or the insulin-like molecules (which three categories are also collectively referred to herein as “insulin-like signals”); and that
2) said one or more biological responses change when (the amount of) the compound(s) to which the nematode is exposed (and/or with which said nematode comes into contact) changes or is altered (for instance, due to a change in the concentration of said insulin like signal in the medium.

The biological response may be any response or combination of responses, such as one or more changes in physiology, biochemistry, development, behaviour, exitation, or other phenotypical properties.

In one particularly preferred embodiment, these may essentially be one or more of the biological responses that are (also) obtained upon (over)expression of insulin the nematode.

One particularly suited biological response may be the dauer-behaviour, e.g. the entry, exit, rescue or bypass of the dauer state, and/or other phenotypical properties that result from and/or are associated with the so-called dauer decision.

In the invention, (one or more strains of) nematodes are used that show increased sensitivity of the insulin pathway, compared to at least the wildtype, and preferably also compared to the reference strain CB1370 (containing the daf-2 reference mutation e1370. This strain is publicly available, for example from the Caenorhabditis Genetics Center (CGC), Minnesota, USA).

By “increased sensitivity of the insulin signalling pathway” is generally meant that the change in the biological response of the nematode (as described above) to a change in (the concentration of) the insulin-type signal is greater than the change that is obtained with the wildtype and/or CB1370 (i.e. for the same change in (the concentration of) the insulin-type signal).

For example, when a change in (e.g. an increase or reduction of) the concentration of an insulin-type signal gives, for the wildtype and/or CB1370, a change in (e.g. an increase or reduction of) the biological response of by a factor of x, than the same change will give, for a strain suitable for use in the invention, a change in the same biological response of more than x (e.g. 1.05 times x, preferably 1.1 times x, more preferably 1.5 times x or even 2 times x or 10 times x, depending on the biological response, the insulin-type signal, the change in concentration, and the specific strain(s) used). In case there is no change observed in wildtype and/or the reference strain CB1370, any change observed determines a strain to be of “increased sensitivity to a insulin-type signal”.

For example, an “insulin-type signal” as used herein may be:
an insulin or insulin-like molecule (e.g. from any suitable source, including but not limited to nematodes, humans or other animals), for which reference is made to PCT/US99/08522, published as WO99/54436 on 28.10.99; Genes & Development 15:672-686, 2001;
a vanadate or a vanadate-type compound, such as sodium orthovanadate;
a PTB-1B inhibitor such as described in Journal of Medicinal Chemistry 43:1293-1310, 25.02.2000, for example compound 66;
wortmannin or a wortmannin-type compound, such as LY 294002 or other PI3-kinase inhibitors.

In this respect, it should be noted that an increase in the concentration of an insulin-type signal may provide an increase in the biological response (in which said increase will be more pronounced for the strain of the invention than for the wildtype and/or for CB1370), or may provide a decrease in the biological response (in which said decrease will be more pronounced for the strain of the invention than for the wildtype and/or for CB1370). For example, an increase in the concentration of a wortmannin will provide an increase in the biological response (for example more dauer), which will be even more pronounced for the strains of the invention (e.g. even more dauer compared to wildtype/CB1370 per increased concentration of wortmannin), whereas an increase in the concentration of a vanadate will provide a decrease in the biological response (for example less dauer), which will be even more pronounced for the strains of the invention (e.g. even less dauer compared to wildtype/CB1370 per increased concentration of vanadate). In case the number of nematodes grown up, i.e. non-dauer, are counted, positive (i.e. increased) and negative (i.e. decreased) biological response are reversed into each other. Both types of insulin-type signals may be used for to determine whether a specific nematode strain has “increased sensitivity of the insulin signalling pathway” compared to wildtype and/or CB1370, and which may be used within the scope of the present invention.

Preferably, the insulin-type signal that is used to determine whether a specific nematode strain has “increased sensitivity of the insulin signalling pathway” is a vanadate-type compound. The vanadate may be used as a free base or as a suitable water-soluble salt, such as sodium orthovanadate. Preferably, the vanadate is used in an amount of between 0.01 and 100 millimolar, more preferably between 0.1 and 10 millimolar, such as 0.5 millimolar or 2.0 millimolar.

Some specific conditions under which vanadates may be used to determine whether a specific nematode strain has “increased sensitivity of the insulin signalling pathway” will be further described below.

Thus, as will be clear from the above, the “insulin-type factor(s)” described above may be used to determine whether a strain has increased sensitivity of the insulin signalling pathway (i.e. compared to the wildtype and/or CB1370) and thus may be used within the scope of the invention.

Generally, such a nematode strain useful in the invention will have “increased sensitivity of the insulin signalling pathway” due to a mutation and/or an other genetically determined factor that provides such increased sensitivity. Such strains will also be referred to below as having a “sensitized genetic background”, and some preferred examples thereof, such as DR1564 and CB1368, will be further described below.

However, it is also within the scope of the invention to provide the strain(s) used with “increased sensitivity of the insulin signalling pathway” by other means, such as exposure to pheromones which increase such sensitivity, by gene suppression techniques such as RNAi, and/or by growing/cultivating the nematodes in the presence of an inducing or suppressing factor (such as population density, food concentration and temperature).

In particular, the nematode strain used may be a weak Daf mutant (i.e. a mutation abnormal in dauer formation), in particular a Daf mutant that is weaker then the reference strain CB1370. For instance, it may be a age-1 mutant, or one of the other daf mutants mentioned herein.

In particular, the nematode strain used may be a weak daf-2 mutant, in particular a daf-2 mutant that is weaker then the reference strain CB1370.

For instance, the reference strain used may be have a Class-I mutation (as mentioned in Gems et al., supra), a mutation which provides a phenotype similar to—and preferably essentially the same as—a Class-I mutation, and/or a(nother) mutation in the ligand binding domain, such that the mutated receptor still has an active kinase domain, but the sensitivity to insulin-like signalling is impaired. However, in its broadest scope, the invention is not limited thereto, and other mutations may also be present, including Class II mutations, as long as the strain having the mutation still has increased sensitivity of the insulin signalling pathway, compared to the wildtype and/or the reference strain C. elegans CB1370.

It is also possible, in the assays of the invention, to use two or more different strains, e.g. one or more which have increased sensitivity of the insulin signalling pathway, and/or one or more references, e.g. wildtype or CB1370.

In one preferred, but non-limiting aspect of the invention, the sensitivity of the insulin signalling pathway of the nematode strain used may be expressed in terms of the “Insulin Sensitivity Value” (ISV), which may be determined in the following manner:

A sample of nematode worms (preferably in the L1 stage) is incubated for between 48 and 96 hours (preferably about 72 hours) separately with and without an insulin-type signal (preferably a vanadate-type compound), at a temperature of between 20 and 25° C. (such as 20, 21, 22, 23, 24 or 25° C.), in the presence of a suitable source of food (such as bacteria, e.g. between 0.05 and 0.5% w/v, preferably about 0.125% w/v), and using a suitable medium (such as S-buffer, M9 or one of the media described in the applications referred to above, and preferably S-buffer).

After incubation, for both the sample with the insulin-type signal and the sample without the insulin-type signal compound, the number of worms in the sample that enter into the dauer state is determined, as a percentage of the number of worms in the original sample, i.e. as follows:
1) for the sample without the insulin-type signal: ([the number of worms that enter the dauer state without insulin-type signal] divided by [the total number of L1 worms in the original sample]) times [100%].
   This percentage is herein referred to as “Percentage A”.
2) for the sample with the insulin-type signal: ([the number of worms that enter the dauer state with the insulin-type signal] divided by [the total number of L1 worms in the original sample]) times [100%].
   This percentage is herein referred to as “Percentage B”.

The Insulin Sensitivity Value may then be expressed as the absolute difference between “Percentage A” and “Percentage B” (i.e. as absolute value of [“Percentage A” minus “Percentage B”]).

As the ISV is calculated as a difference between two percentages A and B, the ISV itself will be a percentage (for instance, when Percentage A is 90%, and percentage B is 10%, the ISV will be 90%-10%=80%), and always positive as the absolute value is calculated (for instance, when Percentage A is 10% and Percentage B is 90%, the ISV will be |10%-90%|=|−80%|=80%.

In the invention, the nematode strain used preferably has an ISV that is greater than the ISV for CB1370. In particular, the nematode strain used may be such that its ISV is more than 1% greater, preferably more than 5% greater, more preferably more than 10% greater, even more preferably more than 20% greater than the ISV for CB1370 (e.g. calculated as the absolute difference between the ISV for the strain used and the ISV for CB1370, e.g. [ISV strain used] minus [ISV CB1370]).

For example, depending upon the specific conditions of the test, CB1370 will usually have an ISV of <20%, more usually <10%, and often <5% (in essence, this means that under the conditions of the test, for CB1370, there is little no difference between the presence and the absence of the insulin type signal). The ISV for wildtype will usually be even lower than the ISV for CB1370.

For the strain used in the invention, under the same conditions of the test, the ISV will usually be >30%, and is preferably >40%, and is even more preferably >50%. (in essence, this means that under the conditions of the test, for the strain used, the difference between the presence and the absence of the insulin-type signal is preferably (much) larger than for CB1370).

Preferably, the ISV is determined using a vanadate-type compound such as sodium orthovanadate, although the invention in its broadest sense is not limited thereto.

Thus, by determining the ISV in the manner outlined above, it can be determined whether a strain has increased sensitivity of the insulin signalling pathway, compared to the wild-type and/or the reference strain CB1370.

Generally, the invention is based on the insight that such nematode strains having increased sensitivity of the insulin signalling pathway can be used with advantage to provide improved methods for the selection of compounds for the field of metabolic diseases, in particular compared to the assay techniques described in PCT US 98/10800 and U.S. Pat. No. 6,225,120. As mentioned above, these methods may be used for drug discovery, development and pharmacology, for instance to discover and/or develop new small molecules and/or small peptides suitable for use in preventing or treating metabolic diseases in human or vertebrates (such as mammals).

For the purposes of the present disclosure, a “small molecule” generally means a molecular entity with a molecular weight of less than 1500, preferably less than 1000. This may for example be an organic, inorganic or organometallic molecule, which may also be in the form or a suitable salt, such as a water-soluble salt.

The term “small molecule” also covers complexes, chelates and similar molecular entities, as long as their (total) molecular weight is in the range indicated above.

In a preferred embodiment, such a “small molecule” has been designed according, and/or meets the criteria of, at least one, preferably at least any two, more preferably at least any three, and up to all of the so-called Lipinski rules for drug likeness prediction (vide Lipinksi et al., Advanced Drug Delivery Reviews 23 (1997), pages 3-25). As is known in the art, small molecules which meet these criteria are particularly suited (as starting points) for the (design and/or) development of drugs (e.g) for human use, e.g. for use in (the design and/or compiling of) chemical libraries for (high throughput screening), (as starting points for) hits-to-leads chemistry, and/or (as starting points for) lead development.

In a preferred embodiment, such a “small molecule” has been designed according, and/or meets the criteria of, at least one, preferably at least any two, more preferably at least any three, and up to all of the so-called Lipinski rules for rational drug design (vide Lipinksi et al., Advanced Drug Delivery Reviews 23 (1997), pages 3-25). As is known in the art, small molecules which meet these criteria are particularly suited (as starting points for) the design and/or development of drugs (e.g) for human use

Also, for these purposes, the design of such small molecules (as well as the design of libraries consisting of such small molecules) preferably also takes into account the presence of pharmacophore points, for example according to the methods described by I. Muegge et al., J. Med. Chem. 44, 12 (2001), pages 1-6 and the documents cited herein.

The term “small peptide” generally covers (oligo)peptides that contain a total of between 2 and 35, such as for example between 3 and 25, amino acids (e.g. in one or more connected chains, and preferably a single chain). It will be clear that some of these small peptides will also be included in the term small molecule as used herein, depending on their molecular weight.

Thus, the methods of the invention may in particular be used to test and/or screen (libraries of) such small molecules and/or peptides, in the manner as further outlined herein.

Thus, in one aspect, the invention relates to the use of at least one nematode worm which has an increased sensitivity of the insulin signalling pathway (compared to the wildtype and/or the reference strain CB1370), in an assay for the identification of a compound, such as a small molecule and/or a small peptide, which is capable of modulating insulin signalling pathways (for example in C. elegans and/or vertebrates, such as humans and/or other mammals), more generally of altering and/or effecting the biological response to insulin signalling, and even more generally for use in (the preparation of compositions for) the prevention and/or treatment of metabolic diseases or disorders (as mentioned above), in vertebrates such as humans or other mammals.

In addition to the identification of small molecules and/or small peptides, according to the inventions, the nematode worms with an increased sensitivity of the insulin signalling pathway may also be used for determining the influence or effect of gene suppression (e.g. by RNAi techniques), and of specific or non-specific mutations (e.g. due to non-specific or (site-)specific mutagenesis).

Preferably, the nematode worm with increased sensitivity of the insulin signalling pathway has a sensitized genetic background (compared to the wildtype and/or the reference strain CB1370), as defined above.

Even more preferably, the nematode worm with increased sensitivity of the insulin signalling pathway (e.g. a sensitized genetic background) has an ISV which is greater than the ISV for wildtype and/or CB1370, and even more preferably an ISV as defined above.

Some preferred, but non limited examples of suitable C. elegans strains include, but are not limited to: DR1564: daf-2(m41), CB1368: daf-2(e1368) and some of the (other) strains mentioned in Gems et al., supra. Other suitable strains will be clear to the skilled person, based upon the disclosure herein.

The most preferred nematode strain is DR1564: daf-2 (m41).

The sample of nematodes may comprise any suitable number of worms, depending on the size of the container/vessel used. Usually, the sample will comprise between 2 and 500, in preferably between 3 and 300, more preferably between 5 and 200, even more preferably between 10 and 100 nematodes. When the assay is carried out in multi-well plate format, each well usually contains between 15 and 75 worms, such as 20 to 50 worms. Although not preferred, it is not excluded that a sample may consist of a single worm.

Usually, each such individual sample of worms will consist of worms that—at least at the start of the assay—are essentially the same, in that they are of the same strain, in that they contain the same mutation(s), in that they are essentially of an isogenic genotype, in that they show essentially the same phenotype(s), in that they are essentially “synchronised” (i.e. at essentially the same stage of development, such as L1 or dauer. It should however be noted that this stage of development may—and usually will—change during the course of the assay, and not for all worms in the sample at the same rate and/or in the same way), in that they have been grown/cultivated in essentially the same way, and/or in that they have been grown under and/or exposed to essentially the same conditions, factors or compounds, including but not limited to pheromones, gene suppression (such as by RNAi), gene- or pathway-inducing factors or (small) molecules, and/or gene- or pathway-inhibiting factors or (small) molecules. However, in its broadest sense, the invention is not limited thereto.

The medium may further contain all factors, compounds and/or nutrients required to carry out the assay and/or required for the survival, maintenance and/or growth of the worms. For this, reference is again made to the prior art, such as the applications and handbooks referred to above. In one specific embodiment, the medium may also contain a suitable source of food for the worms—such as bacteria (for example a suitable strain of E. coli)—in a suitable amount.

In the method of the invention, the sample of nematodes can be kept—e.g. maintained, grown or incubated—in any suitable vessel or container, but is preferably kept in a well of a multi-well plate, such as standard 6, 24, 48, 96, 384, 1536, or 3072 well-plates (in which each well of the multi-well plate may contain a separate sample of worms, which may be the same or different). Such plates and general techniques and apparatus for maintaining/handling nematode worms in such multi-well plate format are well known in the art, for instance from the applications mentioned hereinabove.

The sample of nematodes may be kept in or on any suitable medium—including but not limited to solid and semi-solid media—but is preferably kept in a suitable liquid or viscous medium (e.g. with a viscosity at the temperature of the assay that is equal to a greater than the viscosity of M9 medium, as measured by a suitable technique, such as an Ubbelohde, Ostwald and/or Brookfield viscosimeter).

Generally, suitable media for growing/maintaining nematode worms will be clear to the skilled person, and include for example the media generally used in the art, such as M9, S-buffer, and/or the further media described in the applications and handbooks mentioned hereinabove.

Preferably, the assays of the invention are based on the dauer phenotype as a biological read out, e.g. the entry into, the bypass of and/or the rescue from the dauer state, and/or any other property which results from and/or is associated with the so-called dauer decision.

For instance, an assay based upon entry into/bypass of the dauer state may comprise the following steps:
a) providing a sample of nematode worms (preferably eggs, L1 or L2 worms, and most preferably L1 worms);
b) keeping said sample under conditions such, without the presence of any compound(s) to be tested, at least 50%, and preferably at least 60%, and more preferably at least 70%, even more preferably at least 80%, such as 85-100% of the nematodes present in said sample would enter the dauer state (at least during the time used for the assay, such as at least 1 day, for example 2-4 days—e.g. about 72 hours—as further described below);
c) exposing the sample to the compound(s) to be tested;
d) measuring either the number of worms that enter the dauer state, and/or measuring the number of worms that grow into adults.

Preferably, in such an assay, the conditions used in step b) are such that, in the presence of a reference compound (such as a vanadate compound, e.g. sodium orthovanadate) at a suitable concentration (such as between 0.5 and 2 milliMolar, which is particularly suited for vanadate), the amount of worms that enter the dauer state is at least 10% less (i.e. lower in absolute difference of percentages as also referred to above), preferably at least 20% less, more preferably at least 30% less, than the amount of worms that enter the dauer state without the presence of any such reference compound (at least during the time used for the assay, such as at least 1 day, for example 2-4 days—e.g. about 72 hours—as further described below).

For instance, the conditions used in step b) may be such that, in the presence of a reference compound (such as a vanadate compound, e.g. sodium orthovanadate) at a suitable concentration (such as between 0.5 and 2 milliMolar, which is particularly suited for vanadate), the amount of worms that enter the dauer state is less than 50%, preferably less than 40%, even more preferably less than 30% (at least during the time used for the assay, such as at least 1 day, for example 2-4 days—e.g. about 72 hours—as further described below, and depending on the amount of worms that would enter the dauer state without the presence of the reference), although the invention in its broadest sense is not limited thereto.

An assay based upon rescue from the dauer state may comprise the following steps:
a) providing a sample of nematode worms in the dauer state;
b) keeping said sample under conditions such that, without the presence of any compound to be tested, least 50%, and preferably at least 60%, and more preferably at least 70%, even more preferably at least 80%, such as 85-100% of the nematodes present in said sample would remain in the dauer state (at least for the time of the assay, such as between 1 and 96 hrs, such as between 12 and 72 hours, such as about 24-48 hours);
c) exposing the sample to the compound(s) to be tested;
d) measuring either the number of worms that remain in the dauer state, and/or measuring the number of worms that go out of the dauer state (e.g. become adults).

Preferably, in such an assay, the conditions used in step b) are such that, in the presence of a reference compound (such as a vanadate compound, e.g. sodium orthovanadate) at a suitable concentration (such as between 0.5 and 2 milliMolar, which is particularly suited for vanadate), the amount of worms that remain in the dauer state is at least 10% less (i.e. lower in absolute difference of percentages as also referred to above), preferably at least 20% less, more preferably at least 30% less, than the amount of worms that remain in the dauer state without the presence of any such reference compound (at least during the time used for the assay, such as between 1 and 96 hrs, such as between 12 and 72 hours, such as about 24-48 hours).

For instance, the conditions used in step b) may be such that, (such as a vanadate compound, e.g. sodium orthovanadate) at a suitable concentration (such as between 0.5 and 2 milliMolar, which is particularly suited for vanadate), the amount of worms that remain in the dauer state is less than 50%, preferably less than 40%, even more preferably less than 30% (at least during the time used for the assay, such as between 1 and 96 hrs, such as between 12 and 72 hours, such as about 24-48 hours, and depending on the amount of worms that would remain in the dauer state without the presence of the reference), although the invention in its broadest sense is not limited thereto.

Techniques for distinguishing, in a sample, and preferably in an automated and/or multi-well plate format, the number of adults and/or the number of dauers will be clear to the skilled person and may include visual/manual techniques, and/or the non-visual detection techniques described in the applications referred to above.

In the assays of the invention, each individual sample of nematode worms will generally be exposed to a single compound to be tested, at a single concentration; with different samples (e.g. as present in the different wells of the multi-well plate used) being exposed either to different concentrations of the same compound (e.g. to establish a dose response curve for said compound), to one or more different compounds (which may for instance be part of a chemical library and/or of a chemical class or series, such as a series of closely related structural analogues), or both (e.g. to the same and/or different compounds at different concentrations).

It is also within the scope of the invention to expose the (sample of) nematodes to two or more compounds—at essentially the same time or sequentially (e.g. with an intermediate washing step)—for example to determine whether the two compounds have an effect which is the same or different from both the compounds separately (e.g. to provide a synergistic effect or an inhibitory or competitive effect).

Furthermore, it is within the scope of the invention to use one or more reference samples, e.g. samples without any compound(s) present, and/or with a predetermined amount of a reference compound. The invention also includes the use, in an assay, of two or more samples of nematode worms of different strains, e.g. to compare (the effect of the compound(s) to be tested on) the different strains, in which said different strains may also be reference strains, such as wildtype, N2 or Hawaiian.

In a preferred embodiment, an assay based on dauer entry/bypass is carried out in a multiwell plate format, under the following conditions:
use of a sample of between 2 and 100, preferably between 10 and 80, more preferably between 15 and 60 worms, such as 20 or 50 worms, preferably eggs, L1 or L2, most preferably L1.
a temperature of between 10° C. and 30° C., preferably between 20° C. and 27° C., such as 21, 22, 23, 24, 25 or 26° C., depending on the specific strain used.
   For example, for DR1564: daf-2(m41), usually a temperature of about 21, 22, 23, 24° C. will be preferred, with a temperature of between 21 and 22° C. being particularly preferred.
   For CB1368: daf-2(e1368), usually a temperature of 24, 25 or 26° C. will be preferred, with 25° C. being particularly preferred.
a concentration of the compound(s) to be tested of between 0.1 nanomolar and 100 milimolar, preferably between 1 nanomolar and 10 milimolar, more preferably between 1 micromolar and 200 micromolar, such as about 20 micromolar. The compound may be taken up by the nematodes in any suitable manner, such as by drinking, soaking, via the gastrointestinal tract (e.g. the gut), via the cuticle (e.g. by diffusion or an active transport mechanism), and/or via openings in the cuticle, such as amphid sensory neurons. Generally, the compound will be mixed with or otherwise incorporated into the medium used;
a time of contact with the compound(s) to be tested of between 0.1 minute and 100 hours, preferably between 1 minute and 90 hours, such as about 1 hour to 72 hours. For instance, the sample of nematodes may be contacted with the compound(s) to be tested for only a brief period of time, e.g. between 1 minute and 2 hours, such as between 20 minutes and 1.5 hours, upon which the sample of nematodes may be washed and further cultivated on fresh medium (i.e. without compound), or the sample of nematodes may be contacted with the compound(s) to be tested for essentially the entire duration of the assay (e.g. for 1-3 days or more). For assays involving (the bypass of) dauer formation (e.g. starting from L1), the time of contact will generally encompass two or mores stages of development, and most preferably be between 1 and 4 days, such as about 2-3 days (e.g. 48 to 72 hours).
a (total) time of incubation of the sample of between 0.1 minute and 100 hours, preferably between 1 minute and 90 hours, such as about 1 hour to 72 hours. For assays involving dauer entry/bypass (e.g. starting from L1), the total incubation time will generally encompass two or mores stages of development, and most preferably be between 1 and 4 days, such as about 2-3 days (e.g. 48 to 72 hours);
the use of a liquid or viscous medium (in which viscous is as defined above), such as S-buffer, M9 or one of the other media referred to in the patent applications mentioned above (as referred to above), with S-buffer being particularly preferred.
The presence of a suitable source of food—for example bacteria such as E. coli—in a suitable amount, e.g. between 0.001 and 10% (w/v), preferably between 0.01 and 1%, more preferably between 0.1 and 0.2%, such as about 0.125% w/v, based on the total medium.

Conditions for assays based on dauer rescue are further described below and/or in PCT US 98/10800 and U.S. Pat. No. 6,225,120.

Although the conditions described above are particularly preferred, more generally, according to the invention, the nematode strains with increased sensitivity of the insulin signalling pathway (as further defined above) may be used with advantage in any C. elegans-based assay technique involving and/or relating to insulin-signalling, insulin signal transduction, biological responses to insulin and/or insulin-type compounds, and/or the insulin pathway. These assays may be based on any suitable phenotypical read out, including but not limited to dauer entry, bypass and/or rescue as described above.

Therefore, in accordance with one aspect of the invention, there is provided a method for the identification of a compound which is capable of modulating insulin signalling pathways, which method comprises:
providing C. elegans larvae of a strain of sensitized genetic background to the insulin signalling pathway;
contacting said larvae with a test compound in growth favouring conditions, i.e. including food; and
screening for growth to adulthood, i.e. bypass of or release from the dauer larval state.

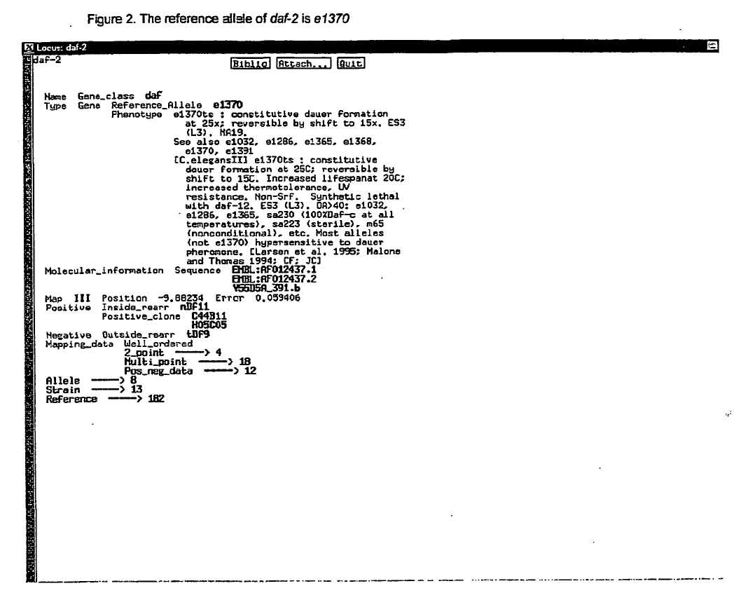

A “sensitized genetic background” may be defined herein by comparison to the reference daf-2 allele, e1370 (FIG. 2 is a print of the acedb database entry on daf-2). The term “sensitized genetic background” encompasses C. elegans strains which exhibits greater sensitivity to test compounds than the daf-2(e1370) allele.

The method of the invention is suitable for use with essentially any C. elegans strain which exhibits a dauer phenotype as a result of defect, for example a mutation, in a gene encoding a component of the insulin signalling pathway or other intervention affecting the insulin signalling pathway and which exhibits a “sensitized genetic background” as compared to the daf-2(e1370) mutant.

In a preferred embodiment the method of the invention may be carried out using C. elegans strain DR1564 containing the daf-2(m41) mutation which exhibit a dauer-constitutive phenotype. Use of strains carrying this allele in compound screens based on bypass of/rescue from dauer is illustrated in the accompanying Examples. Table 6 compares the activity of 94 compounds, which were found to be positive in a primary screen of 8,000 compounds using DR1564: daf-2(m41), as part of Example 1, in a retest on the m41 allele bearing strain DR1564 and on the daf-2 alleles bearing strains CB1368: daf-2(e1368) and daf-2(e1370). DR1564: daf-2(m41) was found to be more sensitive to compound activities than CB1368: daf-2(e1368), with 56% and 27% confirmation rate, respectively. The strain CB1370 containing the daf-2 reference allele e1370 could not be rescued by any of the 94 compounds.

Other sensitized backgrounds in addition to daf-2(m41) may be used in accordance with the invention. Since both m41 and e1368 belong to class I alleles in the classification of Gems et al. 1998, Genetics 150: 129-155, while e1370 belongs to class II, it is likely that other class I alleles are also useful as sensitized genetic background. Typically class I alleles are mutations in the ligand binding domain, and class II mutations are located in the kinase domain. The precise molecular lesion of m41 is unknown.

Other C. elegans strains with sensitized genetic backgrounds which may be used in accordance with the invention include strains exhibiting a dauer phenotype which comprise loss of function or reduction of function mutations in genes downstream of the insulin receptor (daf-2). A particular example is the age-1 mutation, a mutation in the catalytic subunit of the PI3-kinase (see FIG. 1 and table 1). While gain of function alleles of akt-1 or pdk-1 are not able to rescue daf-2(e1370), they do rescue age-1 mutations (Paradis and Ruvkun 1998, Genes & Dev 12:2488-2489, Paradis and Ruvkun 1999, Genes & Dev 13:1438-1452).

While there are no mutations known in the regulatory subunit of the PI3-kinase (located on the yac clones Y119C1 and Y110A7), knock-out mutations in these genes may be generated by methods known by the art (Zwaal et al. 1993, PNAS 90: 7431-35; Liu et al. 1999, Genome Research 9:859-867). Other suitable strains carry loss of function mutations in the genes encoding AKT protein kinases. Since there are two redundantly acting AKT potein kinases (Paradis and Ruvkun 1998, Genes & Dev 12:2488-2489), a double mutation of knock-outs of both akt-1 and akt-2 may be to be constructed by simple crossing. Another potential useful mutation is the loss of function mutation in pdk-1(sa680), as described in Paradis and Ruvkun 1999, above cit.

In a further embodiment of the method of the invention, a C. elegans strain having a sensitized genetic background may be obtained by inhibiting proteins of the insulin-receptor pathway using specific inhibitor compounds. In particular, inhibitors of the PI3-kinase are known, such as Wortmannin and LY294002. Barbar et al. 1999, Neurobiol Aging 20:513-519 demonstrate the activity of LY294002 in inducing dauer formation. The inventors own experiments also illustrate the activity of Wortmannin (FIG. 4).

RNAi inhibition is still another method of generating C. elegans strains with loss of function phenotypes suitable for use in the method of the invention. Methods of inhibiting expression of specific genes in C. elegans using RNAi are well known in the art and described, for example by Fire et al., Nature 391:801-811 (1998); Timmins and Fire, Nature 395:854 (1998) and Plaetinck et al., WO 00/01846. Most preferred are the techniques described in WO 00/01846 which use special bacterial strains as food source to obtain double stranded RNA inhibition.

In yet another embodiment of the present invention, sensitized strains may be used which comprise gain of function mutations of daf-18 or daf-16 or of the C. elegans homologs of PTP-1B or SHIP2. Generation of gain of function mutations of serine or threonine phosphorylation sites, as disclosed for daf-16 by Paradis and Ruvkun 1998, above cit., and by Kops et al. 1999, Nature 398: 630-634, is straightforward for researchers experienced in the state of the art, as demonstrated by Nakae et al. 2000, EMBO 19: 989-996 for FKHR, a human homologue of daf-16.

Yet another sensitized genetic background may be derived by using mutants defective in perception of environmental signals that regulate insulin signalling, such as pheromone, food and temperature signals, or mutations in the neural processing of said signals, or mutations in the secretion of insulin-like molecules or in one of the genes encoding for an insulin-like molecule. In a preferred embodiment tph-1(mg280) is used, a mutant deficient in tryptophan hydroxylase, necessary for serotonin biosynthesis. C. elegans worms with this mutation accumulate large stores of fat and to some extend form dauer larvae because of inability to process the food sensation, together with impaired temperature sensation (Sze et al. 2000, Nature 403: 560-564). Other suitable sensitized genetic backgrounds comprise daf-c mutations in daf-1, daf-4, daf-7, daf-8, daf-11, daf-14, daf-21, daf-19 or daf-28. Furthermore, dominant activation mutations in neuronal G proteins, as described by Zwaal et al. 1997, Genetics 145: 715-727, may also serve as sensitized background.

Several synthetic dauer forming mutations are known, which enhance other genetic backgrounds to form dauer mutations. One specific example, the double unc-64(e246); unc-31(e928), is given by Ailion et al. 1999, PNAS 96, 7394-7397. Since unc-64 encodes for a homolog of syntaxin, a protein involved in synaptic transmission and other types of Ca ²⁺-reulated secretion and unc-31 encodes for a homolog of CAPS, Ca²⁺-dependent activator protein for secretion and insulin release in pancreatic β cells is determined by Ca²⁺-regulated secretion the simplest model is that the Daf-c phenotype of the double mutation is caused by a shut down of release of either insulin like molecules themselves or of neurotransmitters that stimulate insulin release (Ailion et al. 1999, PNAS 96, 7394-7397).

Sensitized worm strains which comprise any combination of two or more synthetic dauer formation mutations amongst each other, or in combination with dauer constitutive mutations, as examples are provided above, or any combination of dauer constiutive mutations with each other may be used in the method of the invention. An example can be drawn from Ogg et al. 1997, Nature 389: 994-999, where a daf-2; daf-1 double mutant induces dauer formation at temperatures far below temperatures necessary for each of the single mutation to induce dauer formation.

The disclosed screening method is based on bypass of/release from the dauer larval state. There are several different ways in which to screen for bypass of/release from the dauer state which may be used in accordance with the invention, as described below. Furthermore, it is possible to use phenotypes of Daf genes other than dauer, including but limited to, fat storage, regulation of metabolic enzymes or stress resistance pathways or any other biochemically, transcriptionally or posttranscriptionally regulated effect that is measurable as the basis of an assay read-out in accordance with the invention.

In accordance with a second aspect the invention also provides a method for the identification of a compound which is capable of modulating insulin signalling pathways, which method comprises:
providing C. elegans larvae of a strain of sensitized genetic background to the insulin signalling pathway;
contacting said larvae with a test compound in growth favouring conditions, i.e. including food; and
screening for growth to adulthood, i.e. bypass of or release from the dauer larval state, wherein conditions of assay are selected such that a basal level of bypass of or release from the dauer larval state is observed in the absence of the test compound.

The second aspect of the present invention comprises of a sensitized assay condition, in contrary to tight screening conditions usually performed in screens to isolate genetic suppressors of daf-2, e.g. daf-16 alleles (Riddle et al. 1981, Nature 290:668-671; Gottlieb & Ruvkun 1994, Genetics 137: 107-120).

The inventors provide a method of setting the assay conditions in way that a basal level of release from the dauer larval state is already present in controls. The basal level of release from the dauer larval state may for example be measured by counting the number of worms growing beyond the dauer stage in a sufficiently large number of control wells (containing the solvent alone but no test compounds). The basal level of release from the dauer larval state will preferably be between 0.1% and 60% rescue, more preferably between 1% and 50% rescue and most preferably between 2% and 40% rescue, such as 10% to 20% rescue. While the minimal number of growing worms or residual activity is derived from sensitizing the assay conditions, the maximal number is derived from experience to optimise signal to noise ratio.

Although in a preferred embodiment the method of the invention uses the temperature sensitivity of daf-2 mutations, such as m41, to sensitize assay conditions, any set of conditions that sensitize the assay over the strict genetic screen conditions is within the scope of the invention, in particular conditions that show growth between 0.1% and 60%, preferentially between 1% and 50%, most preferentially between 2% and 40%, such as 10% to 20%, in cases where the readout of the assay is related to bypass of or release from the dauer-constitutive phenotype.

Another embodiment of the invention uses genetic means to sensitize assay conditions to the desired basal level of release from the dauer larval state. For example Ogg & Ruvkun (1998), Mol. Cell 2: 887-893, disclose a double mutation daf-2; daf-18, which gives rescue (L4 and adults) at a level of 2.2%. In addition, mutations known as Daf-d for dauer defective, especially weak mutations, can be used in the present invention. Also gain of function mutations, as there are known pdk-1(mg142), (Paradis and Ruvkun 1999, Genes & Dev 13:1438-1452) and akt-1(mg144), (Paradis and Ruvkun 1998, Genes & Dev 12:2488-2489), can be used to rescue from dauer formation to a certain percentage. Furthermore, gain of function, in particular at phosphorylation sites, or loss of function mutations can be generated by methods known in the art (see citations in the section further above).

Also suitable for use in the method of the invention are C. elegans strains which comprise a mutation in a gene downstream of the insulin receptor in the insulin signalling pathway which leads to a reduction in the function of the product of the mutated gene but not a complete loss of function. Residual activity of the product encoded by the gene mutated in such strains may be sufficient to confer a basal level of release from the dauer larval state.

Another embodiment of the invention comprises the incomplete loss of function typically seen with RNAi experiments. Since the disclosed methods rely on growth of worms in presence of E. coli, methods of obtaining RNA inhibition via feeding of appropriately engineered bacterial strains may be used as discribed in Plaetinck et al., WO 00/01846.

Still another embodiment of the invention comprises incomplete rescue typically obtained by heterologous transgenes. For example, a strain daf-16; daf-2; Ex[daf-16b::hsFKHR] has been constructed in which daf-16 loss of function, in itself rescuing from daf-2 induced dauer formation, is rescued by the human homolog FKHR under the C. elegans daf-16b promoter. This rescue is incomplete, to about 60% dauer formation, so that 40% grow to adulthood (Gary Ruvkun, personal communication). Any other homologue of daf-16, for example the human genes FKHRL1 or AFX, or others, mammalian or human, could be used in combination of suitable promoters, either one of the endogenous daf-16 promoters, daf-16a or daf-16b or both, or a heterologous promoter, preferably with ubiquitous expression or nervous system expression.

Still another embodiment of the invention is based on the addition of pheromone preparations so that the fraction of worms growing adults is driven below 60%, preferably below 40%, more preferably below 40%, such as between 10% and 20%. As already mentioned, Sze and co-workers (Nature 403: 560-564) generated a tph-1(mg280) mutation, which induces dauer arrest at 15%, mimicking low food supply and with some resistance to temperature control. However, since the dauer arrest can be enhanced to 80% using a daf-7 mutation, which are defective in production of a TGFβ like molecule signalling the absence of pheromone, addition of pheromone could achieve the desired level of 80% dauer formation as an alternative to the double mutant. Pheromone preparations may be obtained after the method of Golden & Riddle 1984, PNAS 81: 819-823.

This screening method of the invention is again based on bypass of/release from the dauer larval state and there are several different ways of screening for bypass of/release from dauer which may be used in accordance with the invention, see below. The invention can as well be based on any other phenotype relating to the insulin pathway, such as are observed in daf-2 mutations, including but not exclusive to fat storage, regulation of metabolic enzymes or stress resistance pathways or any other biochemically, transcriptionally or posttranscriptionally regulated effect that is measurable.

Set out below are ways of screening for bypass of or release from the dauer larval state which may be used in accordance with the invention.

One of the simplest and most exact methods of, measuring bypass of/rescue from dauer larvae formation is counting of adults. Counting of adults may be achieved using automated means, e.g. automatic plate readers, allowing the screen to be performed in mid-to-high throughput format in multiwell microtiter plates.

A further method of screening for bypass of or rescue from the dauer phenotype exemplified herein is based on staining of adults using Nile Red an automated data acquisition (Example 2). Other methods of screening for release from the dauer larval state are also encompassed by the invention.

As an alternative to direct counting of adults indirect measurements, for example the consumption of food by measuring turbidity, may form a usable readout.

Further methods of screening for bypass of/release from the dauer larval state are based on the use of reporter transgene. Suitable reporter transgene constructs generally comprise a promoter or promoter fragment operably linked to a reporter gene. The promoter or promoter fragment is one which is capable of directing strong gene expression in adult C. elegans but no or weak gene expression in dauer larvae, such as a promoter which is regulated by the daf-2 signalling pathway (e.g. promoters regulated by the transcription factor daf-16) or vice versa (i.e. no or weak expression in adult, strong expression in dauer larvae. The term “operably linked” refers to a juxtaposition in which both components function in their intended manner, i.e. the promoter drives expression of the reporter gene. One example of a suitable transgene is a construct comprising the C. elegans vit-2 promoter operably linked to a luciferase reporter gene. Any other promoter that shows strong expression in adults but no or weak expression in dauer larvae may be used as an alternative to the vit-2 promoter. Other reporter genes may be used as alternatives to luciferase. Preferably the reporter gene will be one encoding a product which is directly or indirectly detectable in the worm, for example a fluorescent, luminescent or coloured product, e.g. GFP or lacZ. Preferably expression of the reporter gene product in the worm will be measurable using an automated plate reader.

The inventors provide methods for constructing ctl-1::luciferase and a sod-3::luciferase reporter transgenes, the ctl-1 and sod-3 genes encoding respective a cytosolic catalase with markedly increase expression in daf-2 dauer larvae (Taub et al. 1999, Nature 399:162-166) and a manganese superoxide dismutase strongly up-regulated in daf-2 mutant adults (Honda and Honda 1999, FASEB 13: 1385-1393). The regulation of a mitochondrial manganese superoxide dismutase by daf-2 is of particular interest, since it has recently been shown that overexpression of a Mn-SOD in vascular endothelial cells can suppress several pathways involved in hyperglycaemic damage, indicating that those damages are caused by production of superoxides (Nishikawa et al. 2000, Nature 404: 787-790).

To perform a screen using a reporter transgene the transgene must first be introduced into the C. elegans used in the screen. This may be achieved using standard techniques for the construction of transgenic C. elegans well known in the art and described, for example, in Methods in Cell Biology, Vol 48, Ed. H. F. Epstein and D. C. Shakes, Academic Press.

TABLE 1
targets of the insulin receptor pathway

| | Human | | | Desired |
|---|---|---|---|---|
| Targets | homologs | Function | Validation | intervention |
| DAF-2 | IR | Receptor tyrosin | e1391 equals het. mutation of | + |
| | | kinase | an morbidly obese diabetic | |
| | | | patient | |
| | PTP-1B | Protein tyrosin | Mouse k.o. insulin | B |
| | | phosphatase | hypersensitive | |
| DAF-2 | IRS-1, -2 | Insulin receptor | IR/+; IRS-1/+ age onset | + |
| | | substrate | diabetes, IRS2 diabetic | |
| AGE-1 | p110 | PI3-kinase | p110β insulin responsive | + |
| | | catalytic | | |
| | | subunit | | |
| | p85/p55 | PI3-kinase | p85α k.o. insulin | +/B |
| | | regulatory | hypersensitive | |
| | | subunit | | |
| DAF-18 | PTEN | PI-3′ | maternal and zygotic minus | B |
| | | phosphatase | rescues daf-2(e1370) | |
| | SHIP2 | PI-5′ | Overexpression inhibits AKT | B |
| | | phosphatase | activation | |
| PDK-1 | PDK1 | AKT | gf rescues dauers, lf induces | + |
| | | phosphorylation | dauers | |
| AKT-1, | AKT = PKB | Forkhead TF | gf rescues, double RNAi | + |
| AKT-2 | | phosphorylation | induce dauers | |
| DAF-16 | FKHR, | Transkription | lf rescues daf-2 (e1370) | B |
| | FKHRL1 | factor | | |

The present invention will be further understood with reference to the following Experimental examples, together with the accompanying Figures in which:

FIG. 1 illustrates the insulin receptor signalling pathway of C. elegans.

FIG. 2 is a print of the acedb database entry on daf-2.

FIG. 3 is a graph to show that vanadates can rescue the genetic insulin resistance caused by daf-2 mutations in C. elegans in an assay based on bypass of/rescue from the dauer larval state.

FIG. 4 is a graph to show that wortmannin further enhances insulin resistance caused by daf-2 mutations in C. elegans in an assay based on bypass of/rescue from the dauer larval state.

FIG. 5 scatter plot of mean and variance of controls for the screening experiment described in Example 1(a) screening, (b) DRC.

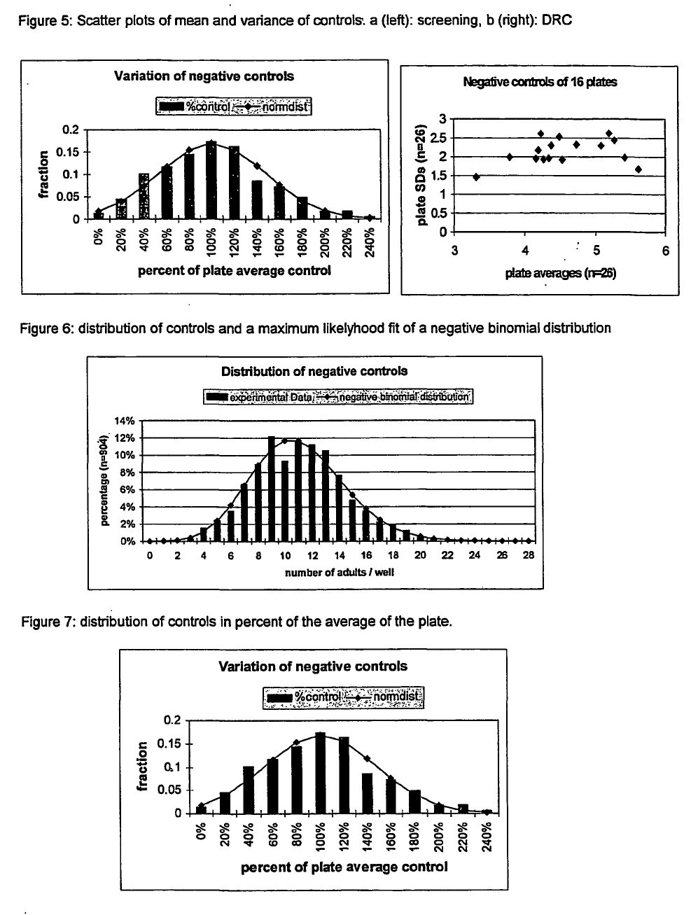

FIG. 6 shows distribution of controls and a maximum likelihood of fit of a negative binomial distribution for data generated in the screening experiment described in Example 1.

FIG. 7 shows distribution of controls in % of the average of the plate for data generated in the screening experiment described in Example 1.

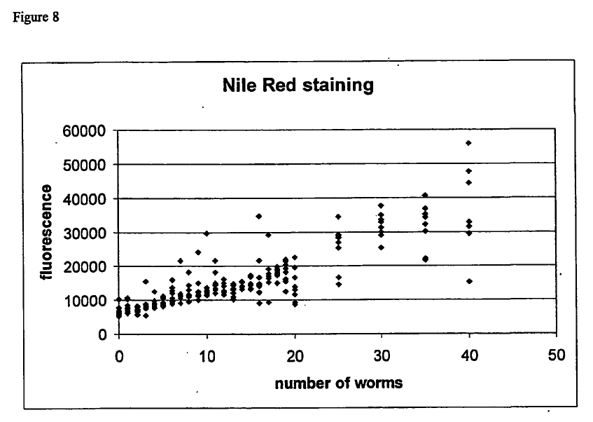

FIG. 8 shows the results of a representative nile red staining experiment (Example 2).

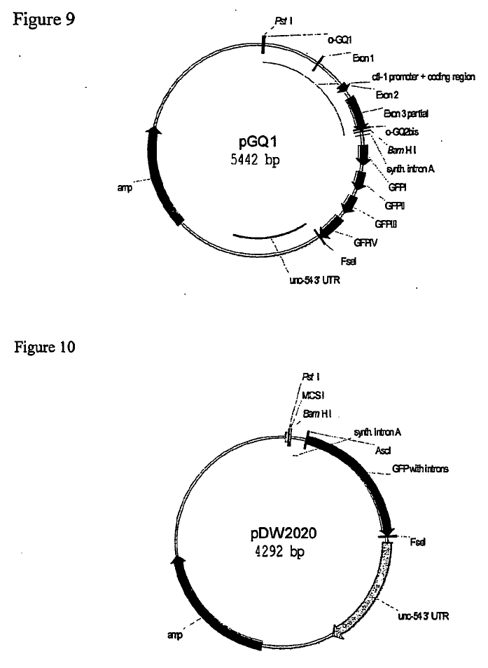

FIG. 9 is a representation of pGQ1.

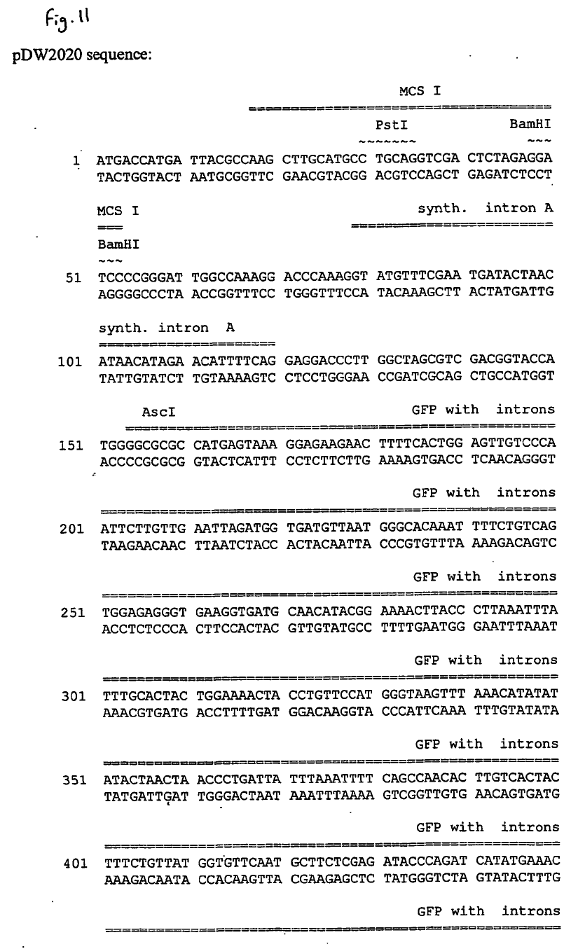

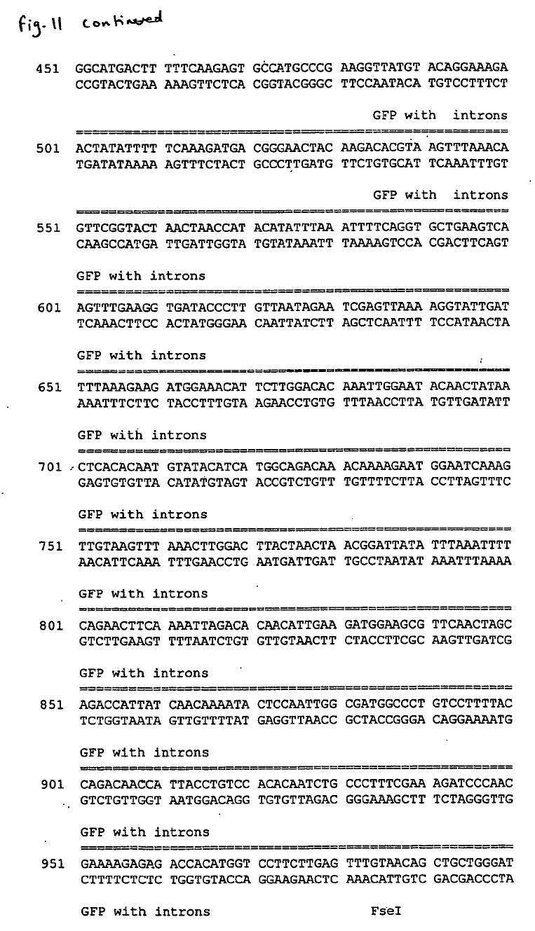

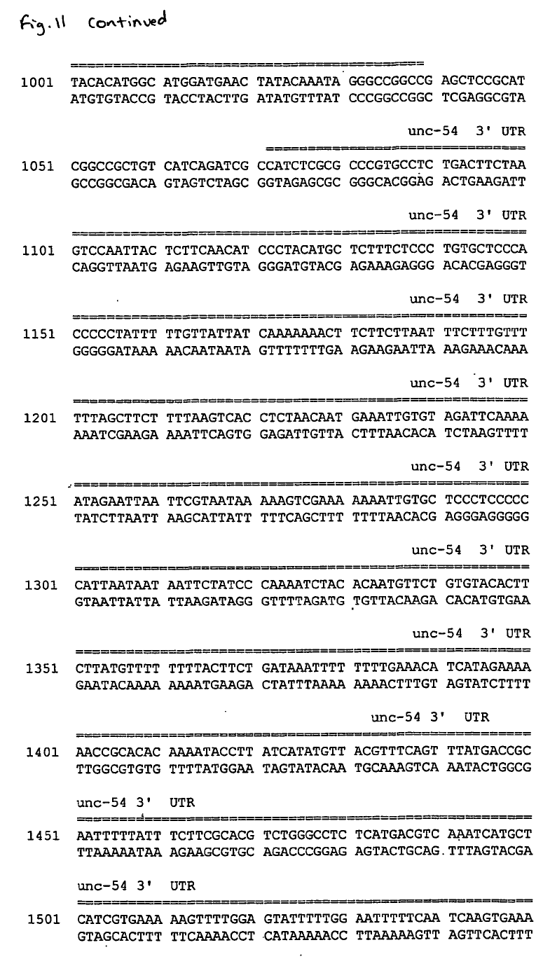

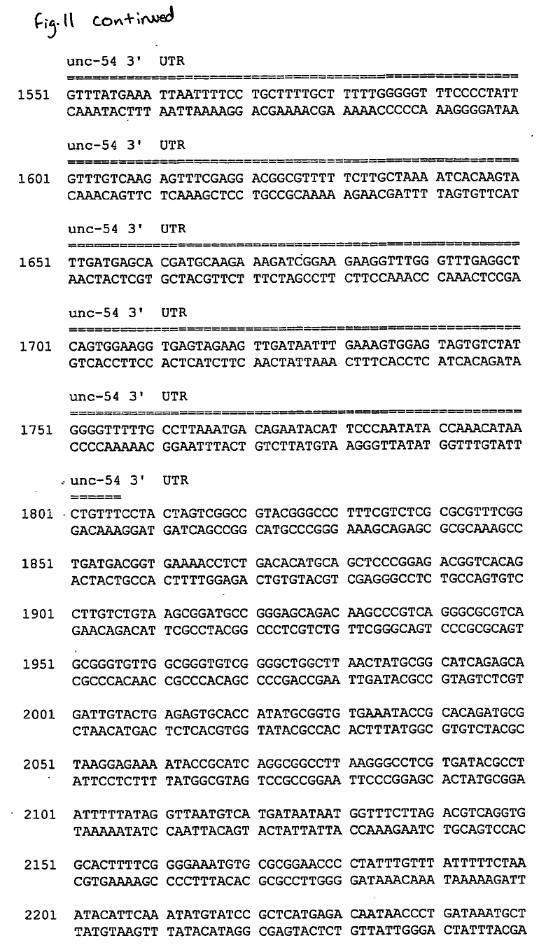

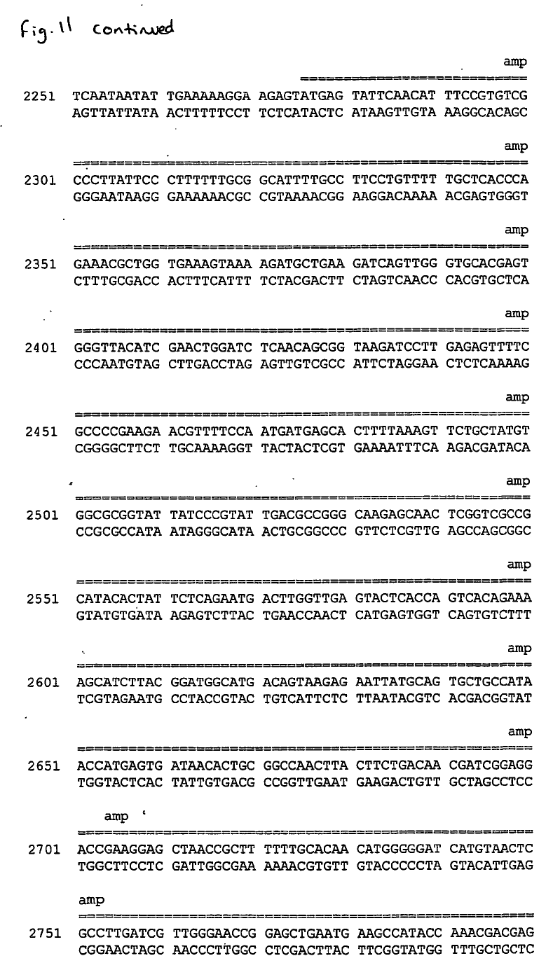

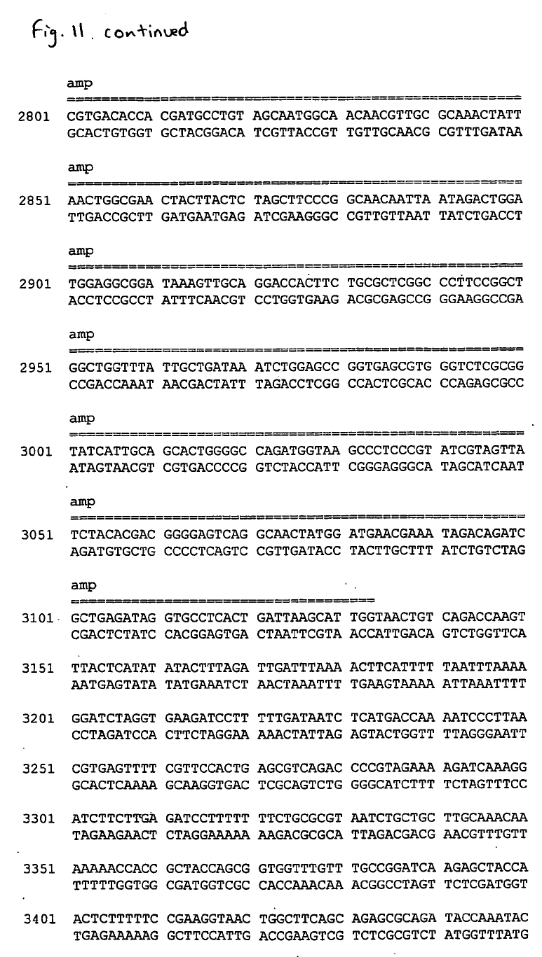

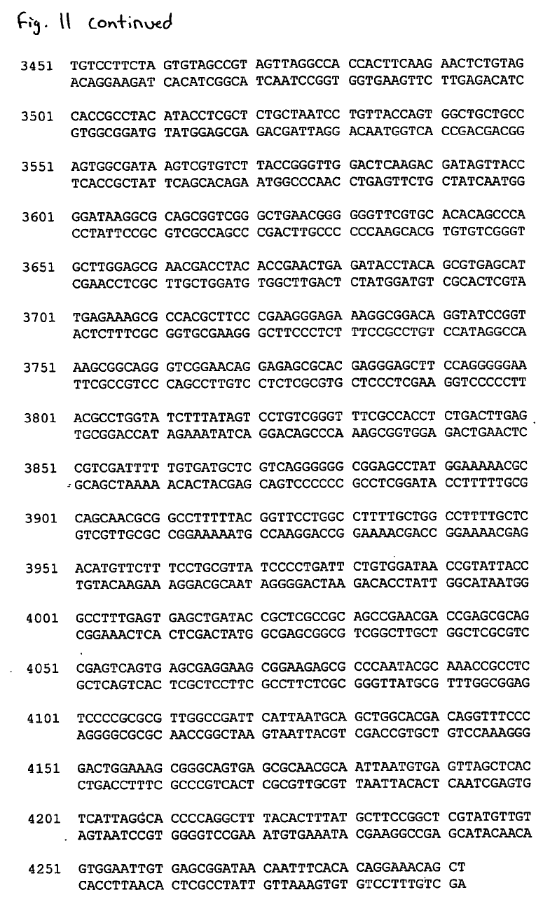

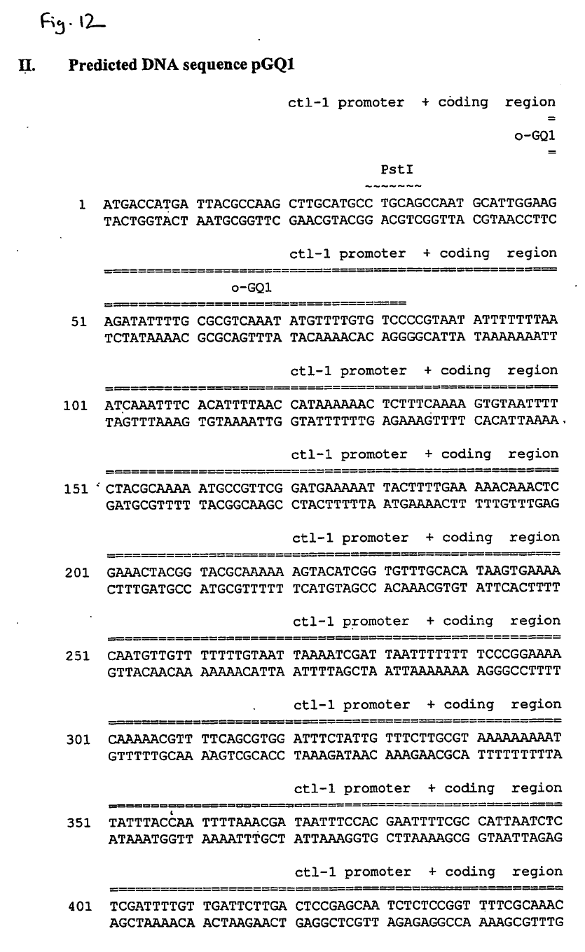

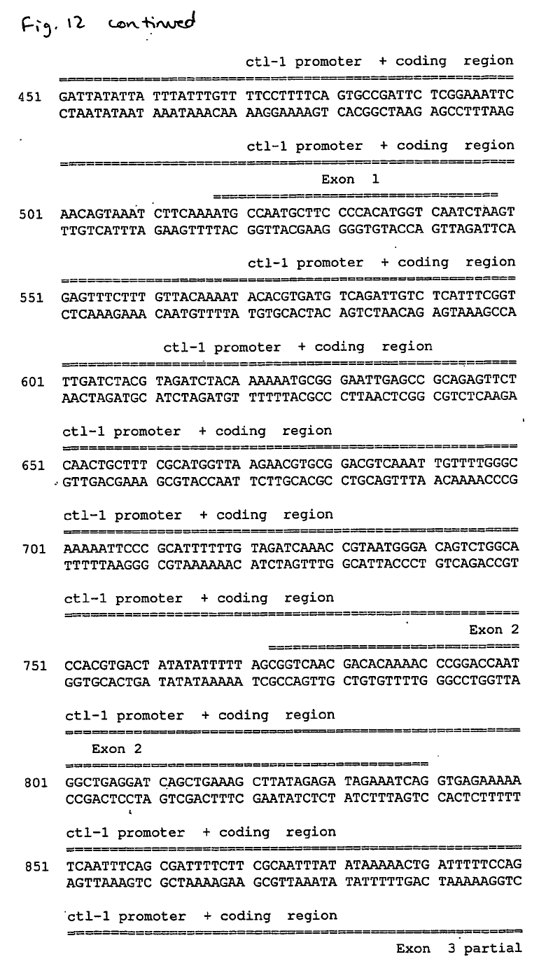

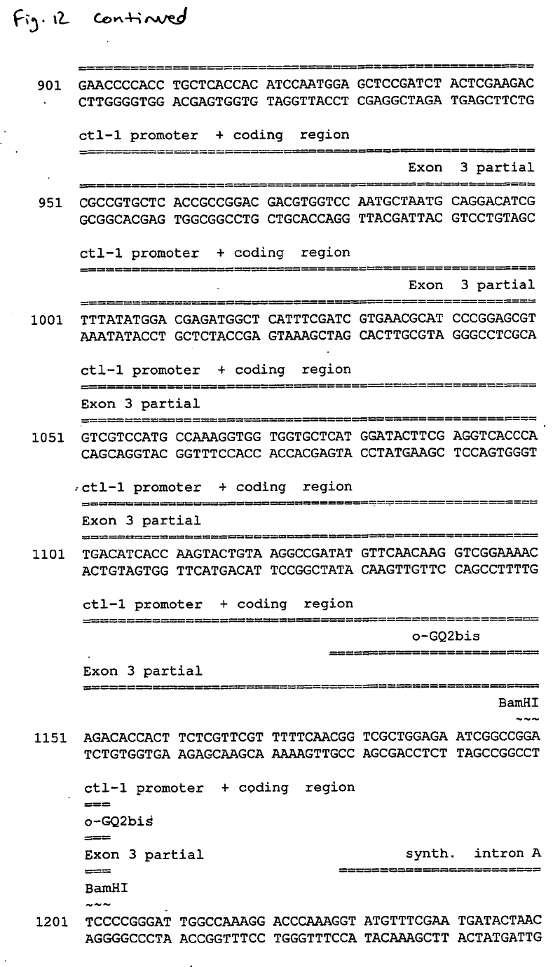

FIG. 10 is a representation of pDW2020.

FIG. 11 shows the complete nucleotide sequence of pDW2020.

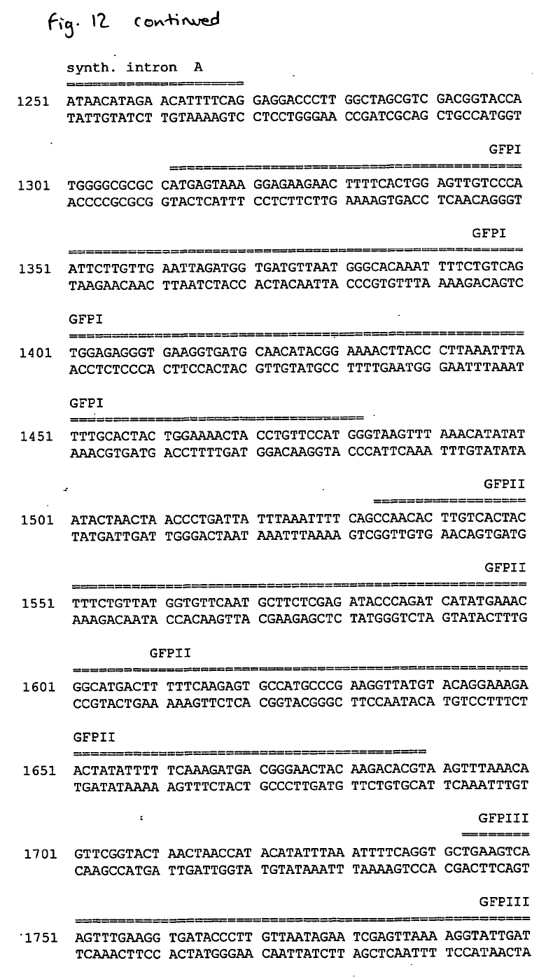

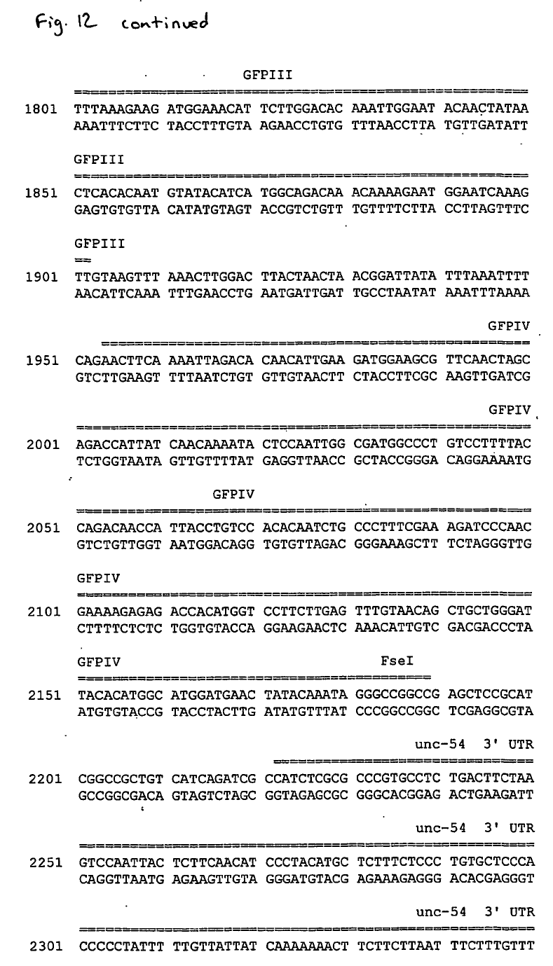

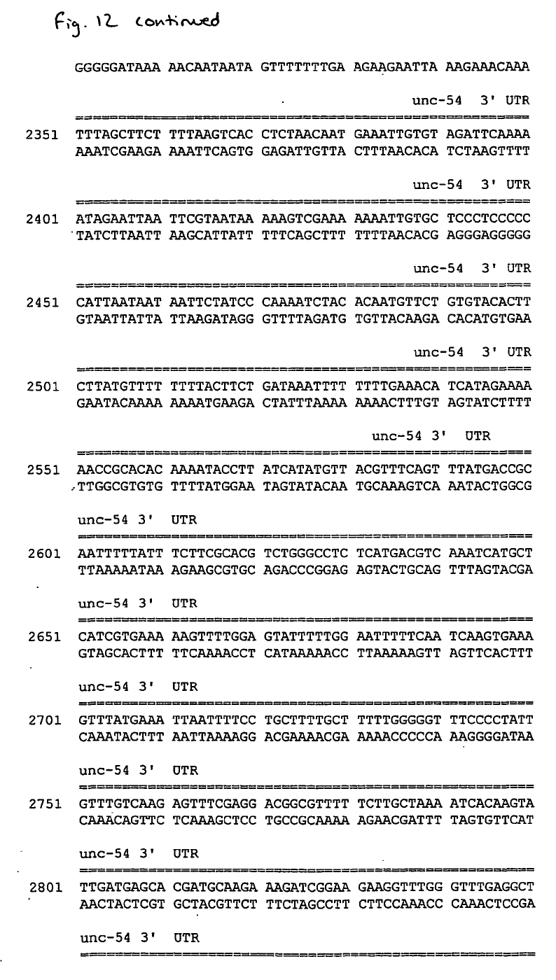

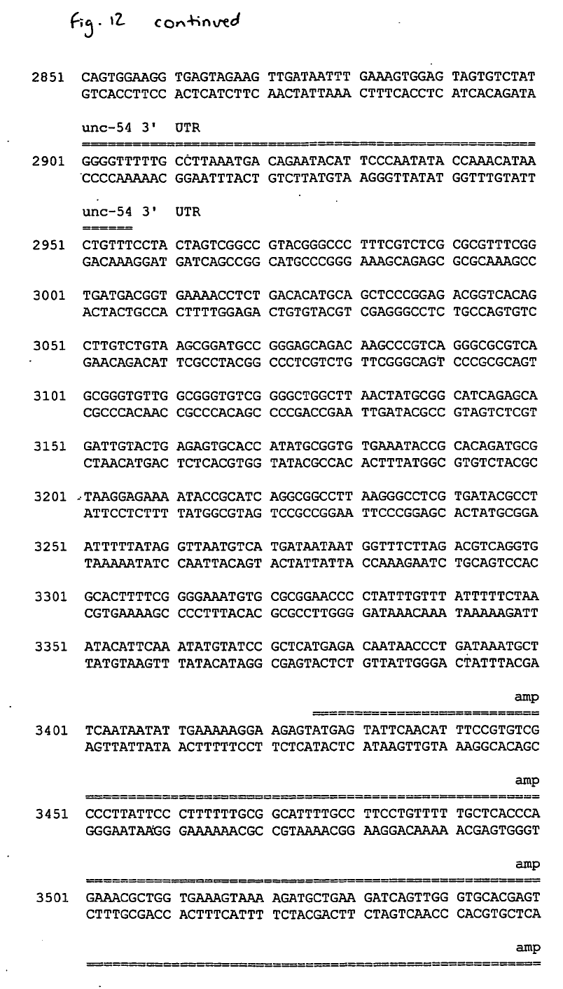

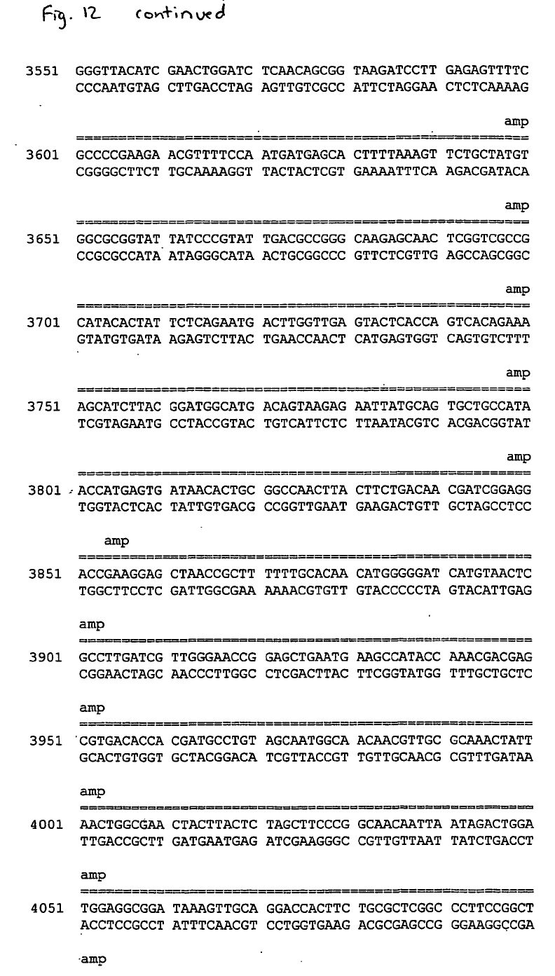

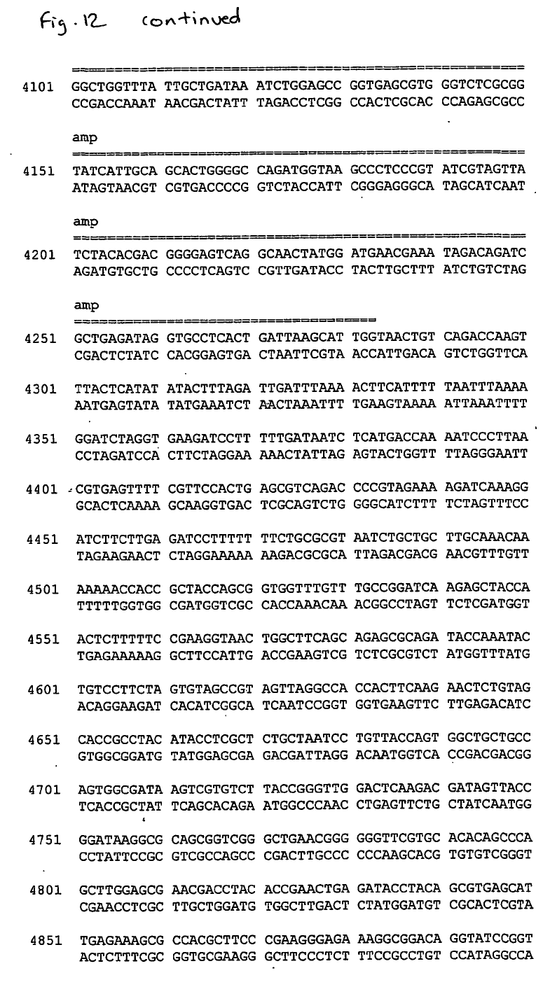

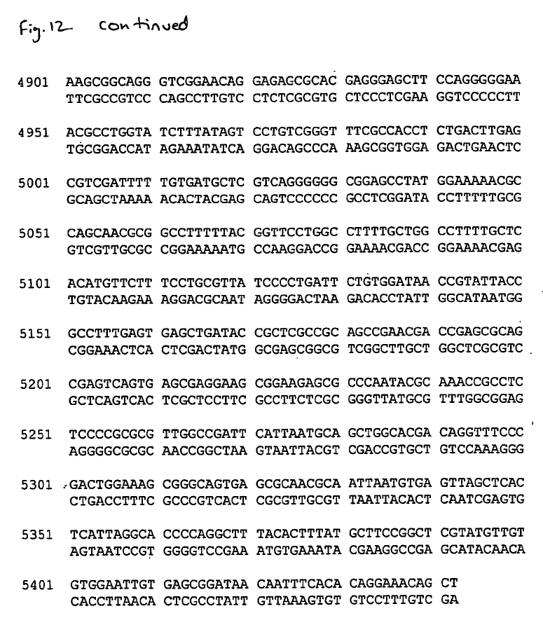

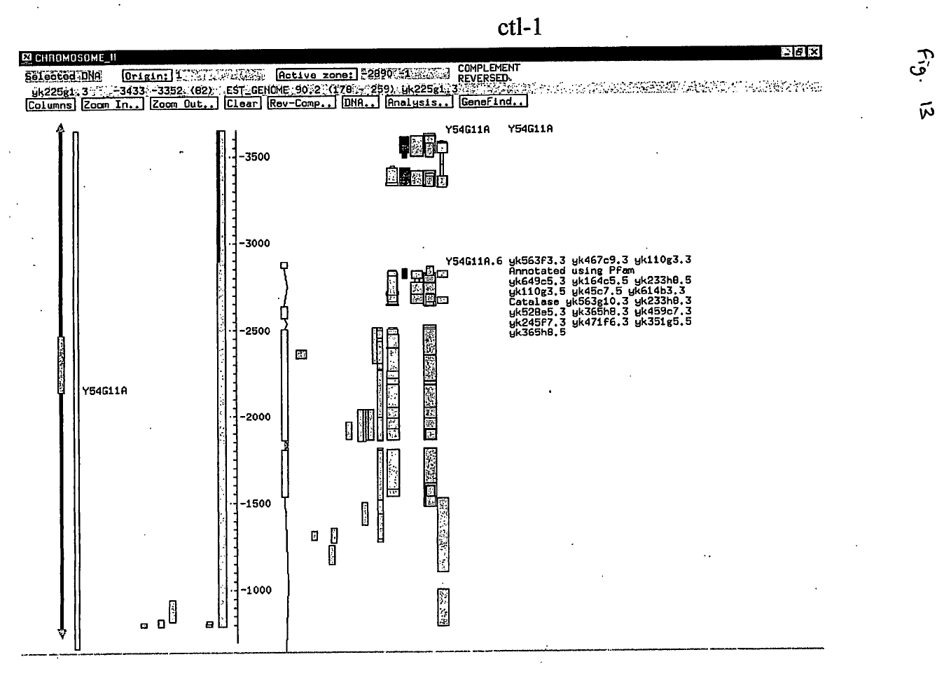

FIG. 12 shows the complete nucleotide sequence of pGQ1.

FIG. 13 is a print of the acedb database entry on ctl-1.

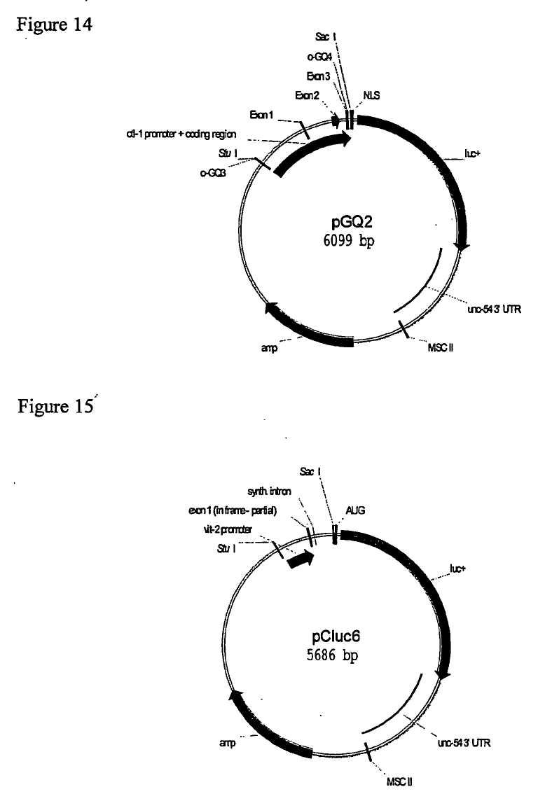

FIG. 14 is a representation of pGQ2.

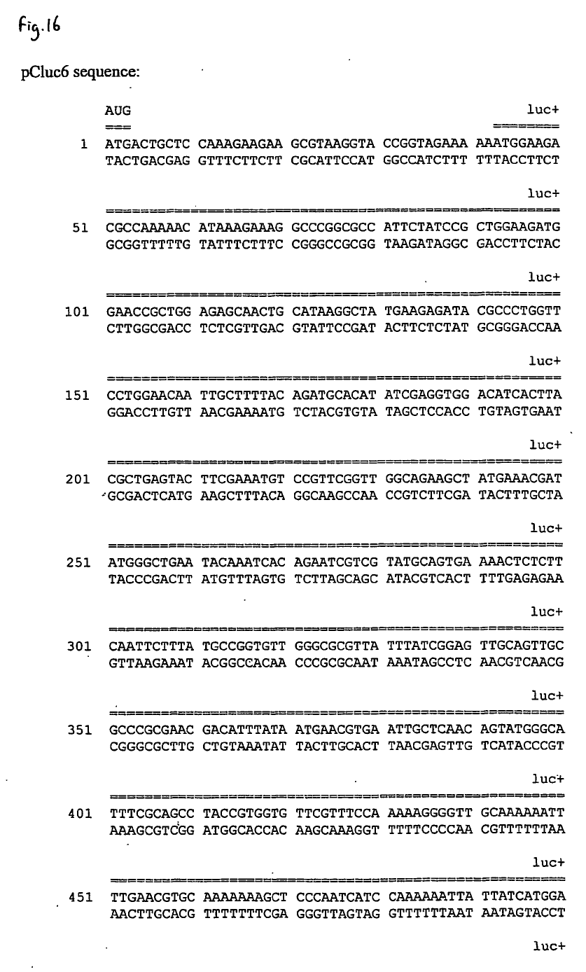

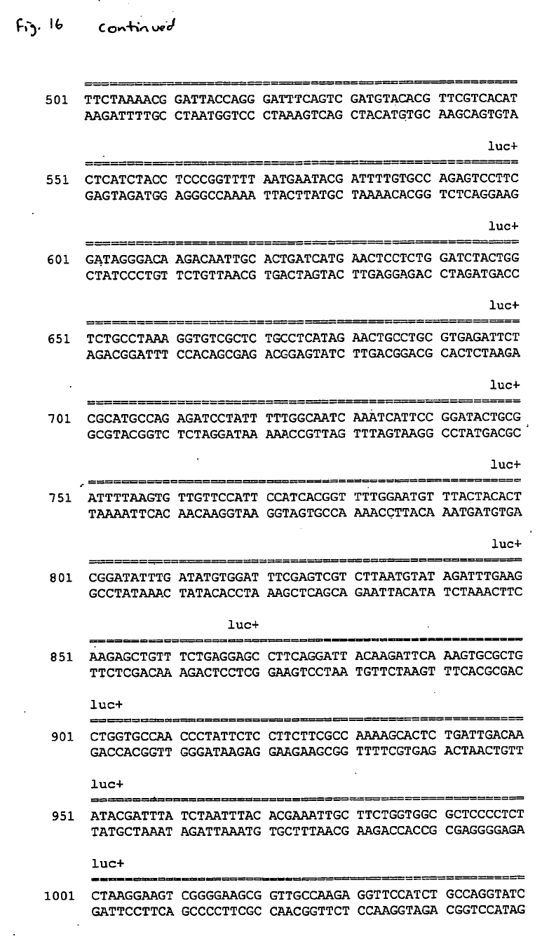

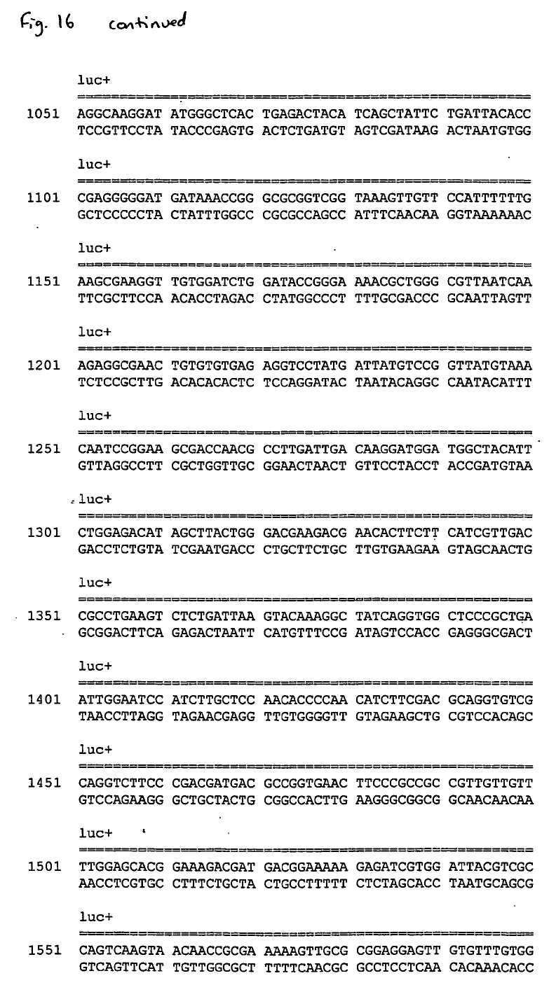

FIG. 15 is a representation of pCluc6.

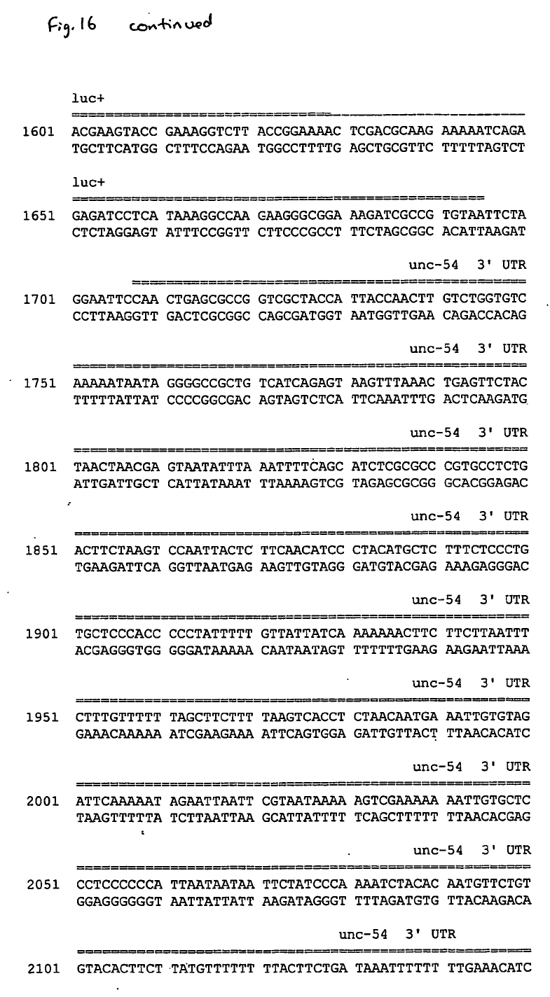

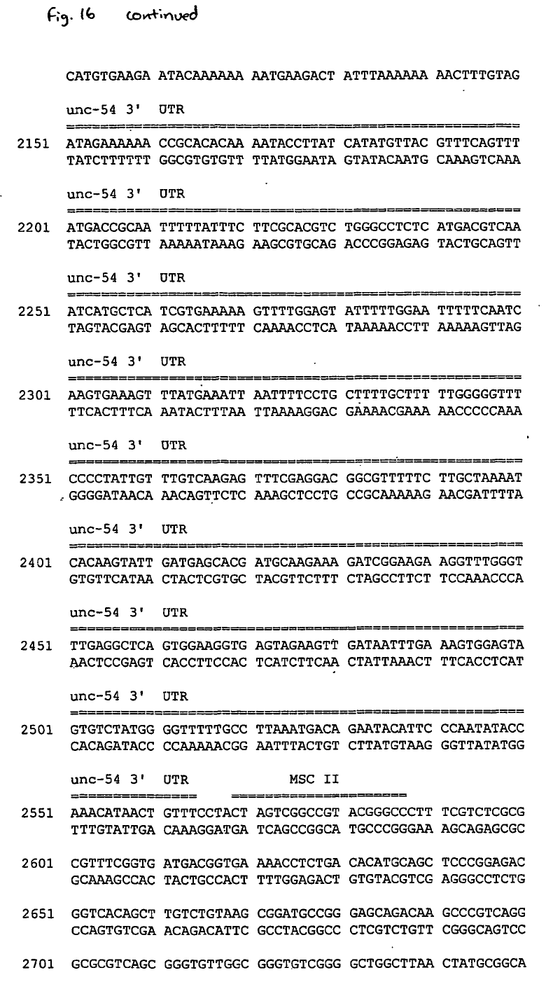

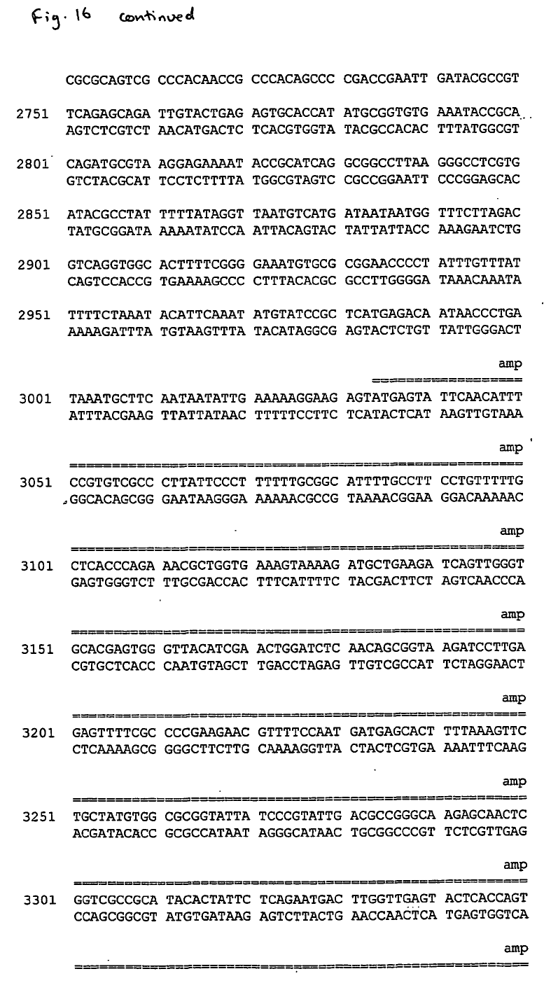

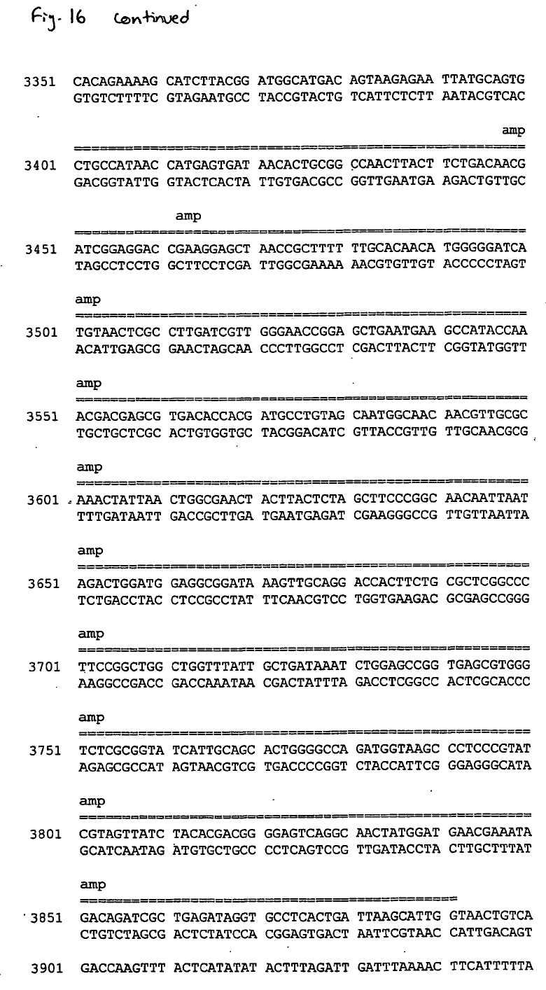

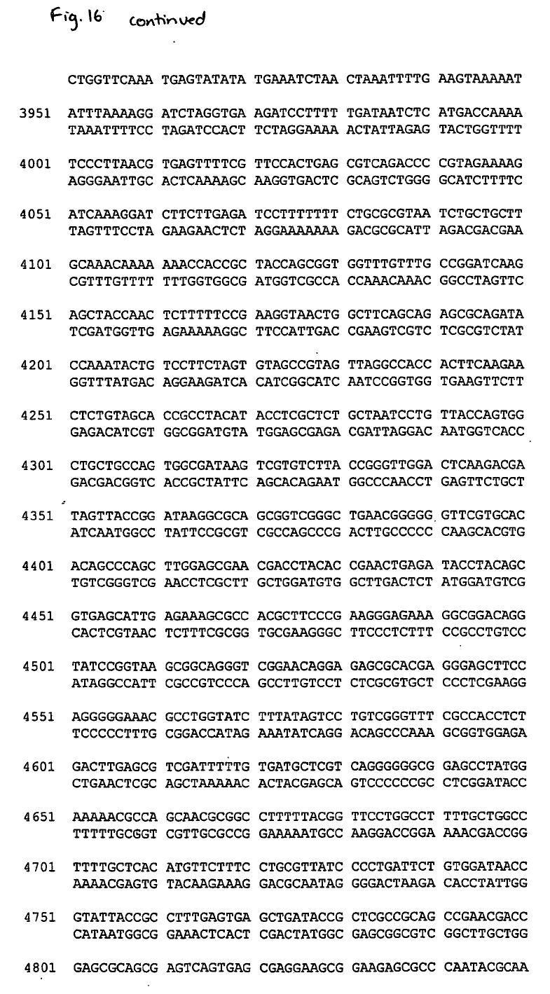

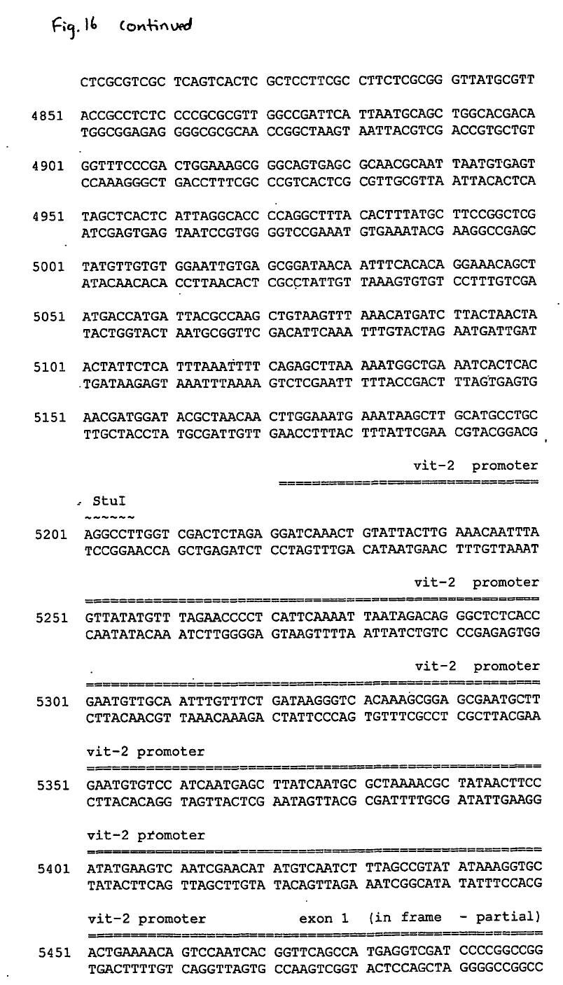

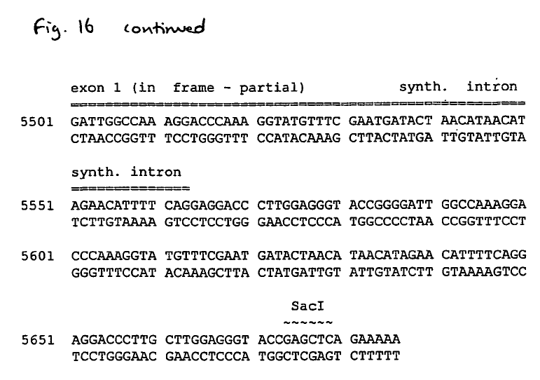

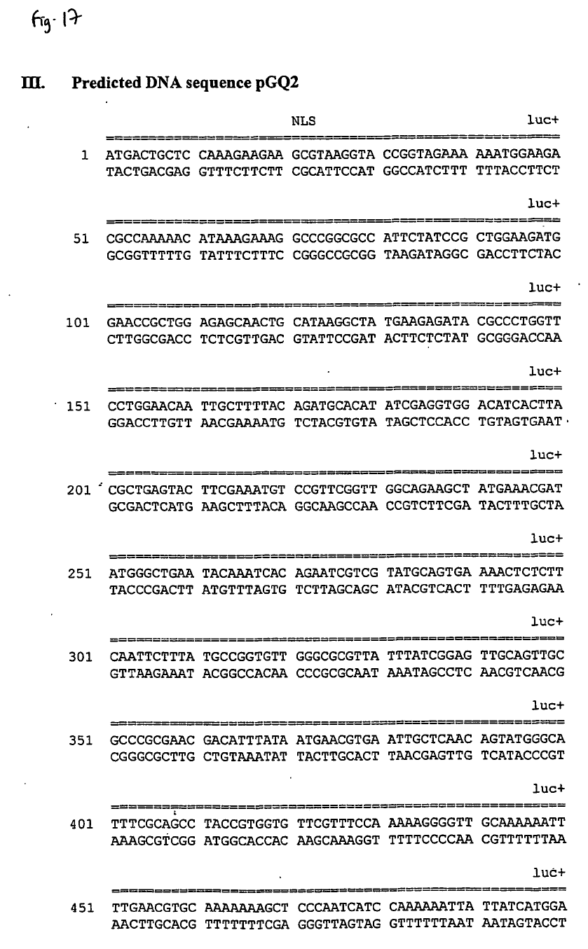

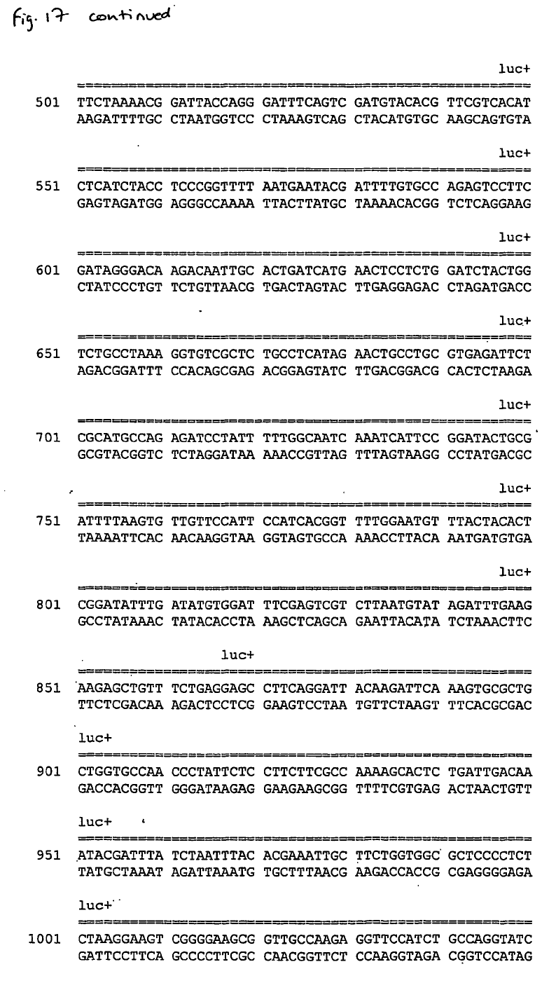

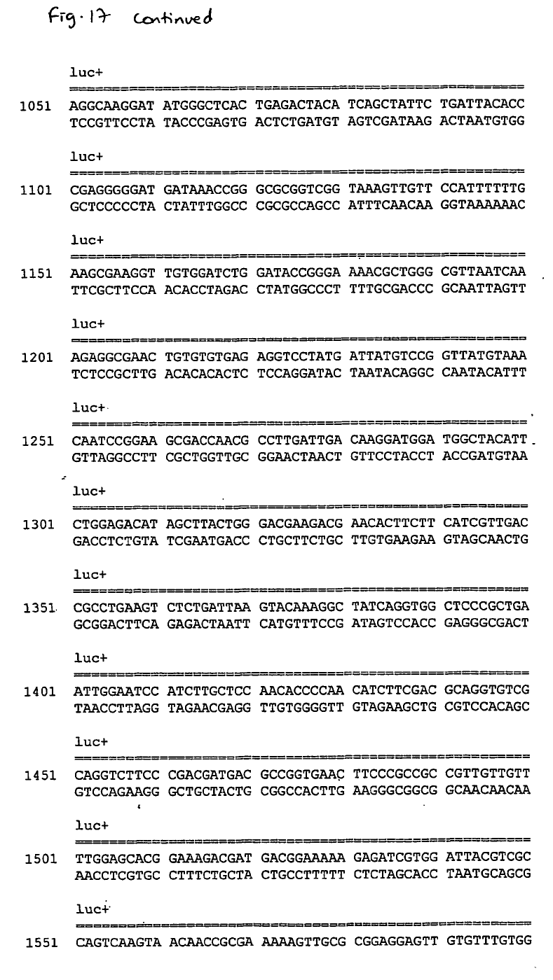

FIG. 16 shows the complete nucleotide sequence of pCluc6.

FIG. 17 shows the complete nucleotide sequence of pGQ2.

FIG. 18 is a print of the acedb database entry on sod-3.

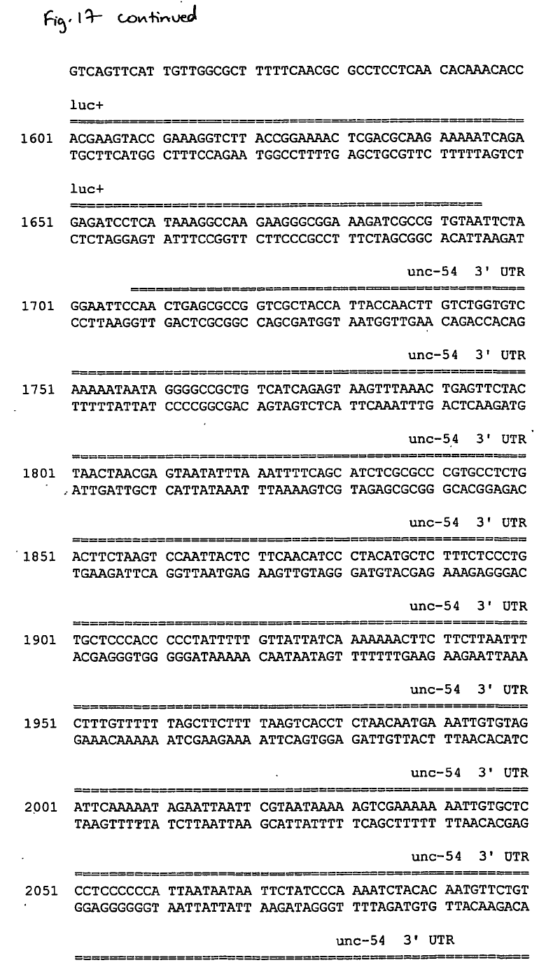

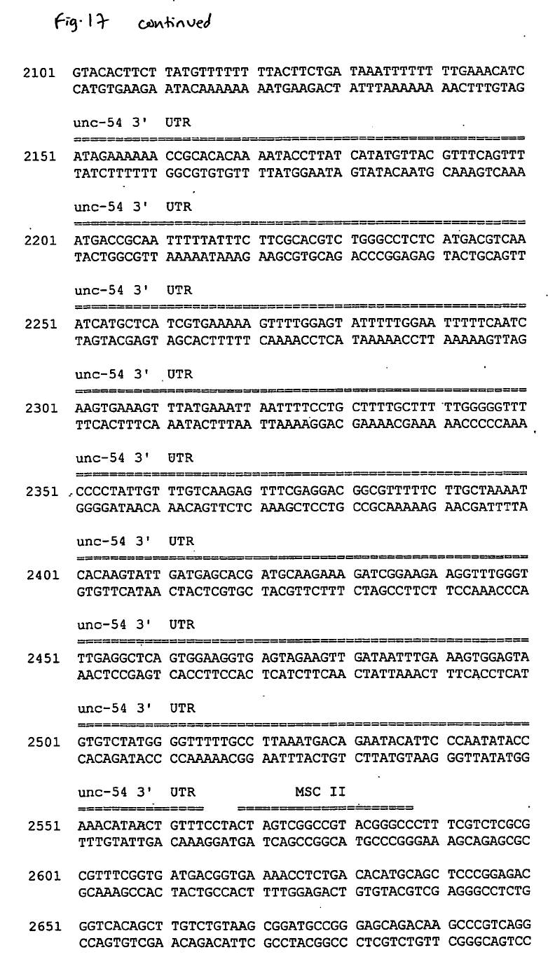

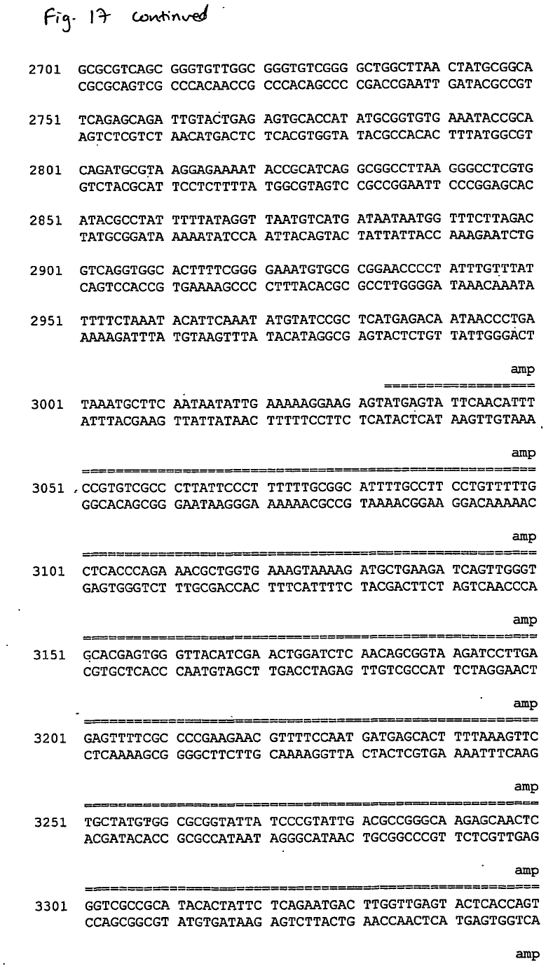

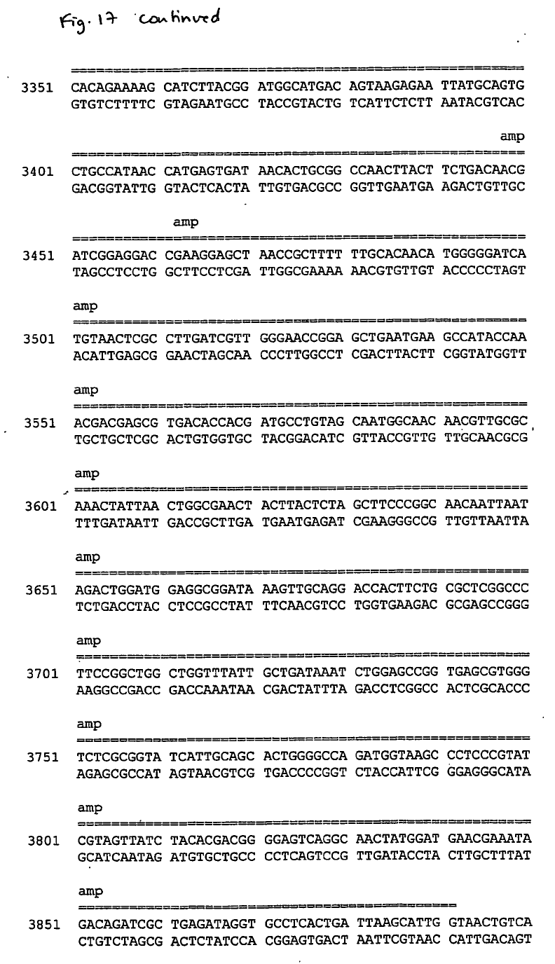

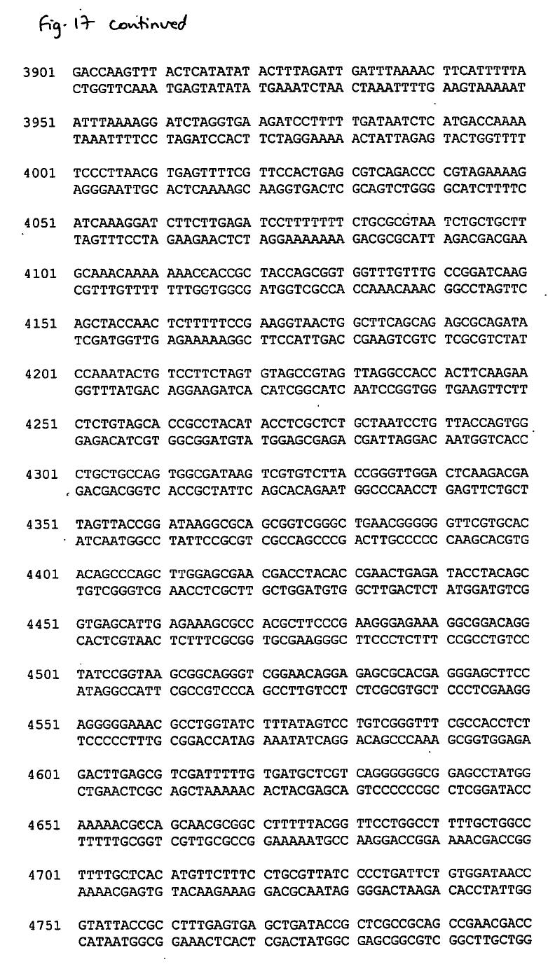

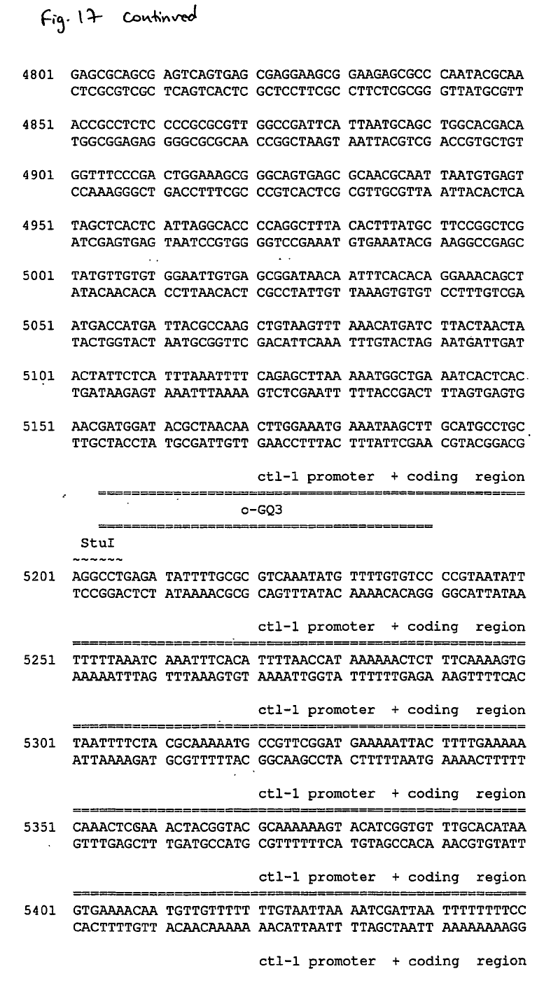

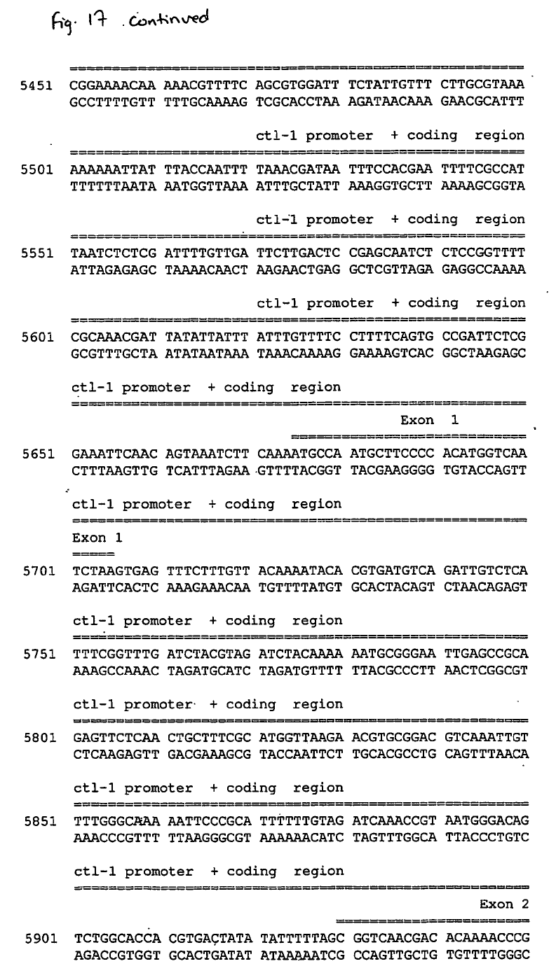

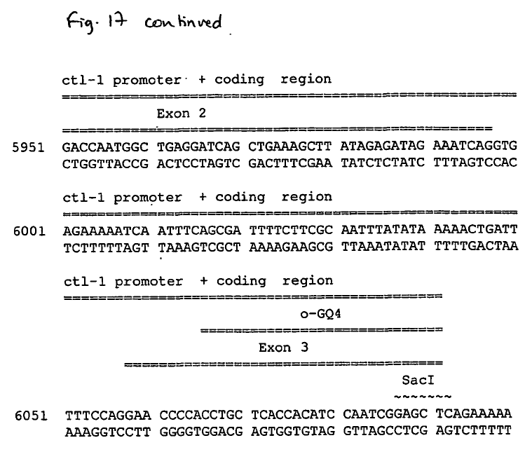

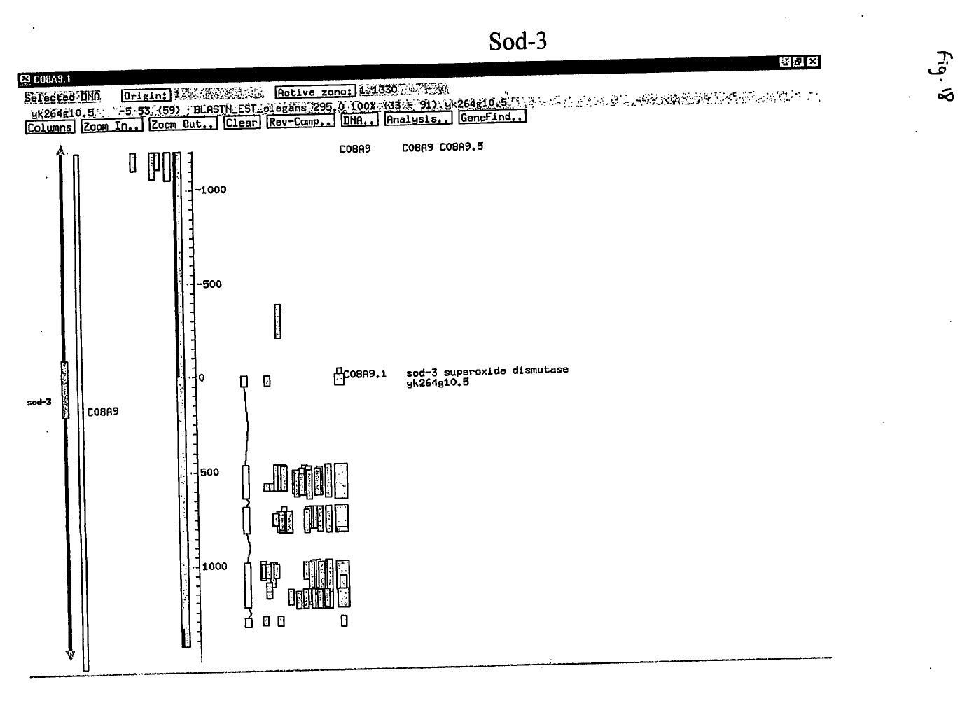

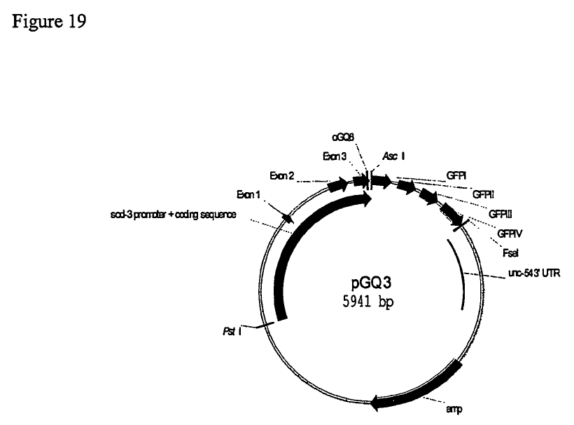

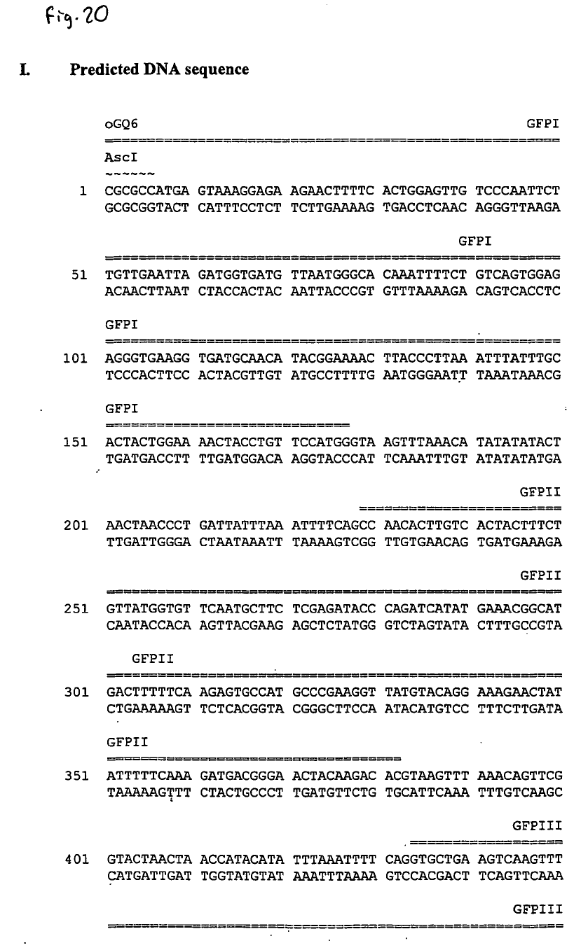

FIG. 19 is a representation of pGQ3.

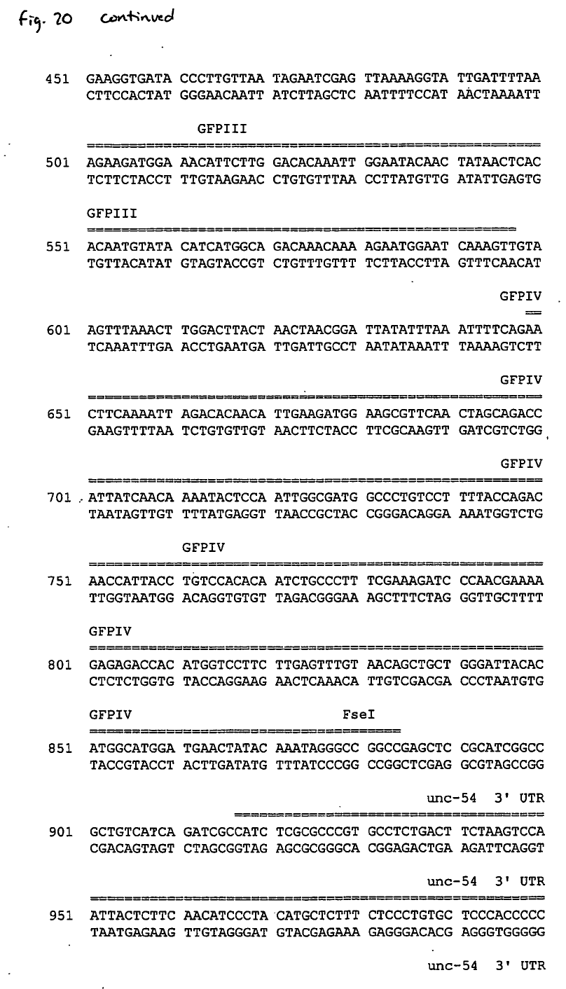

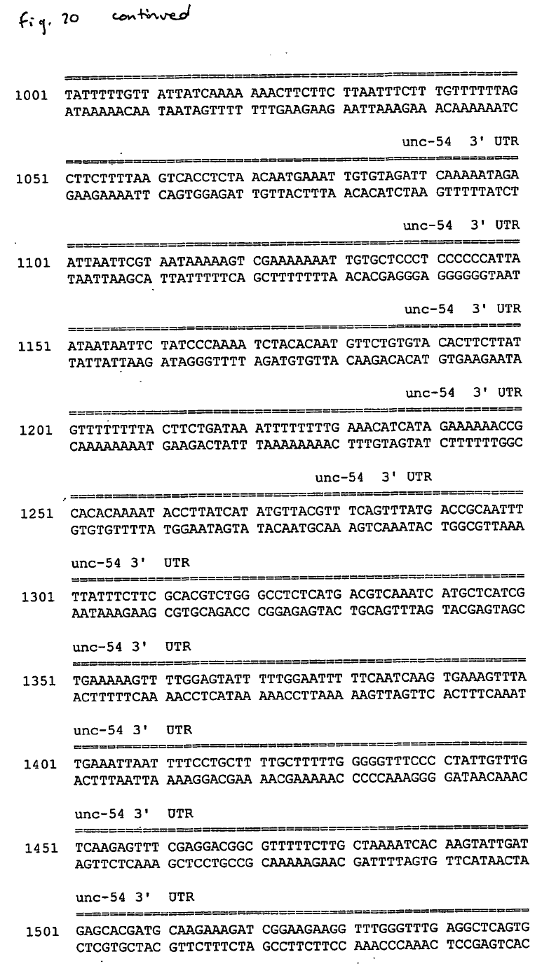

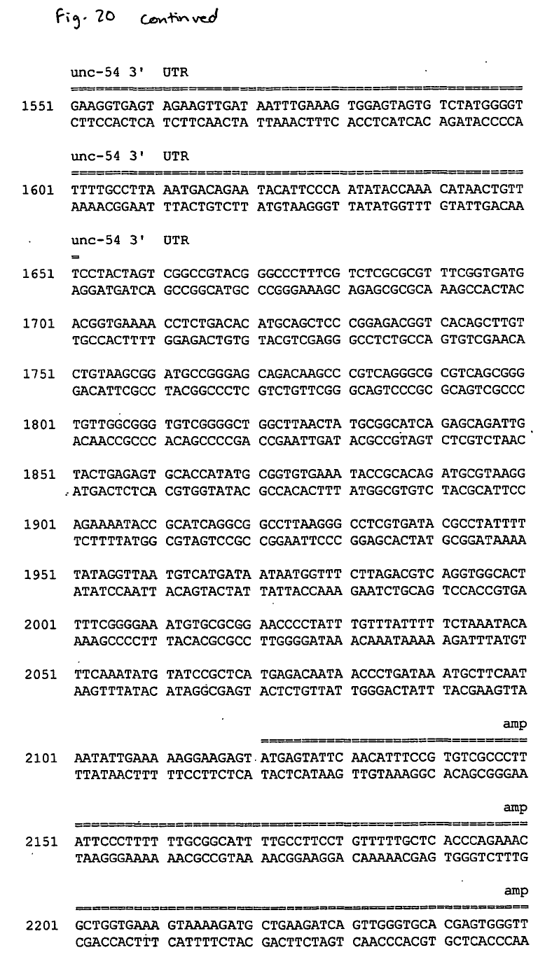

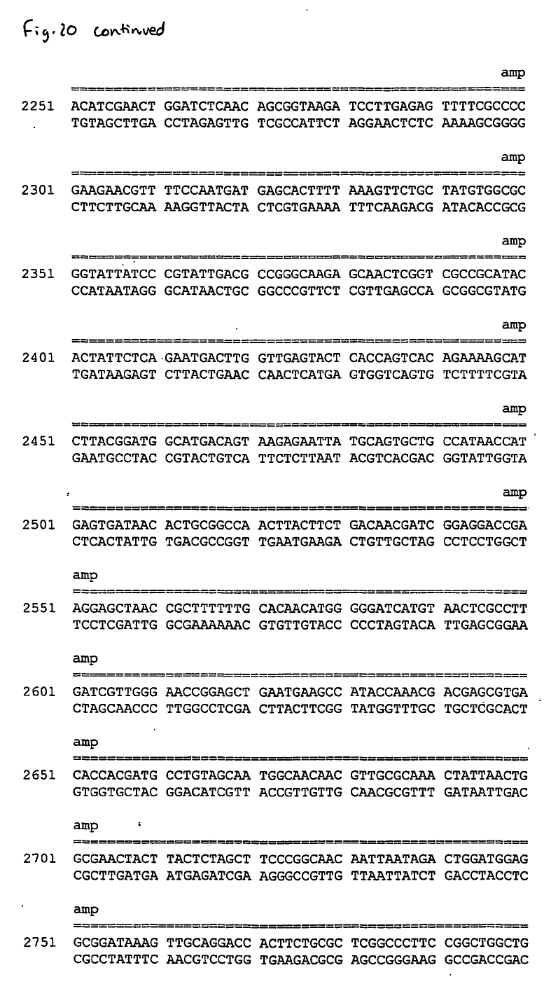

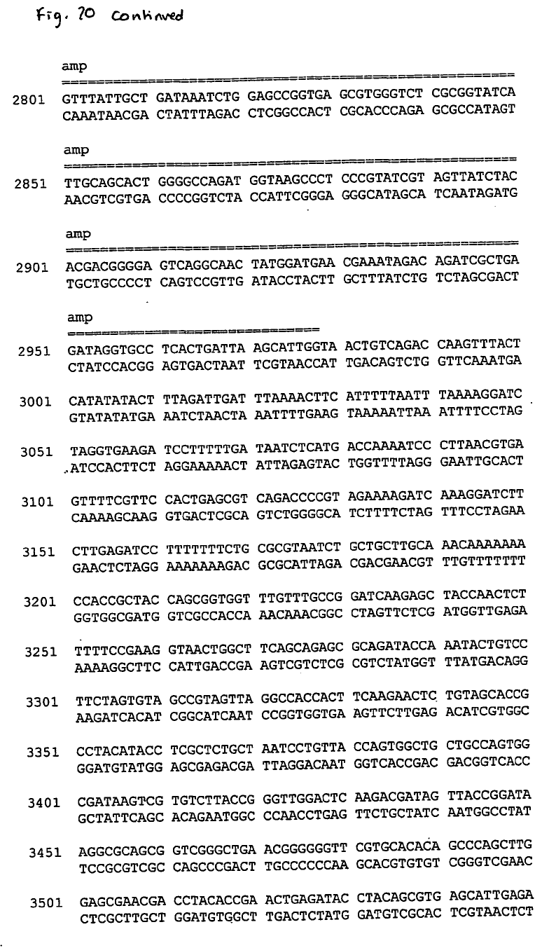

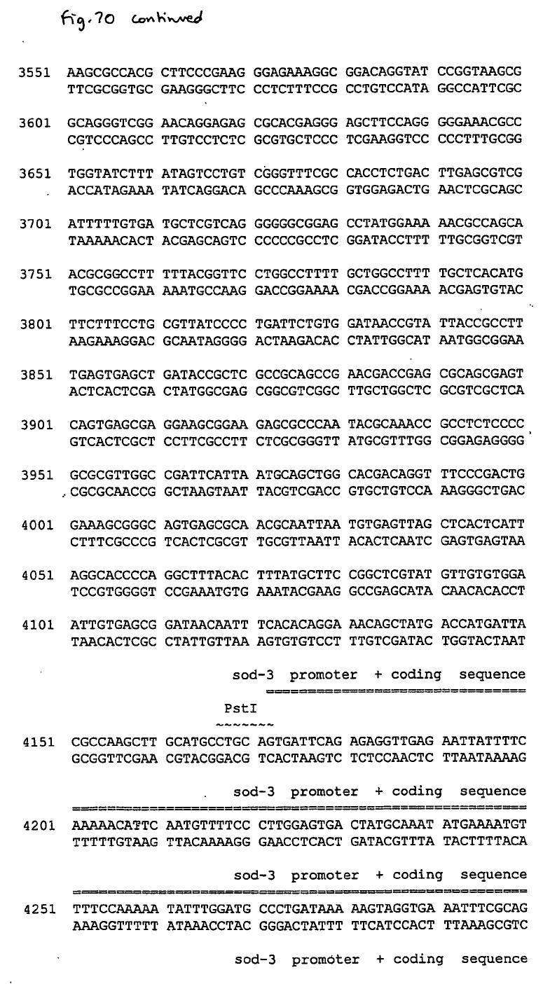

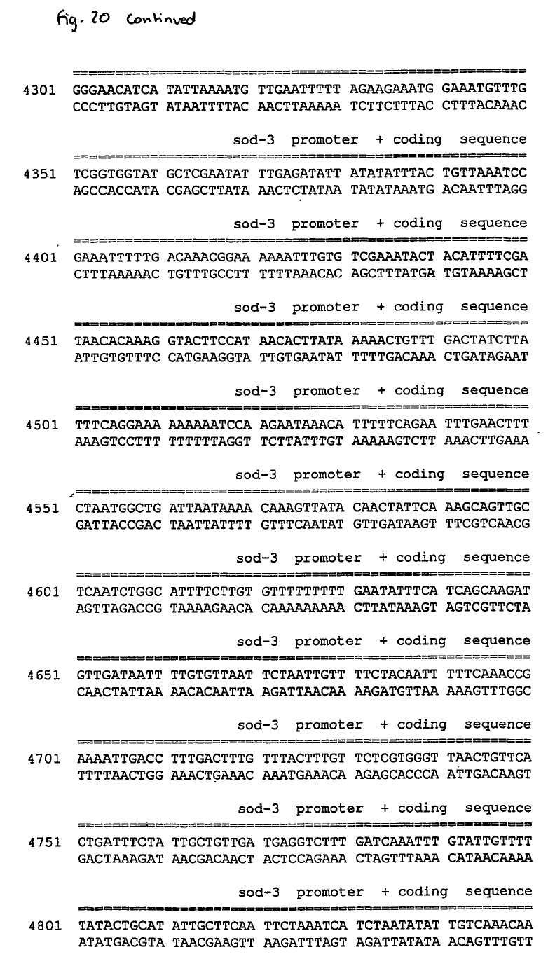

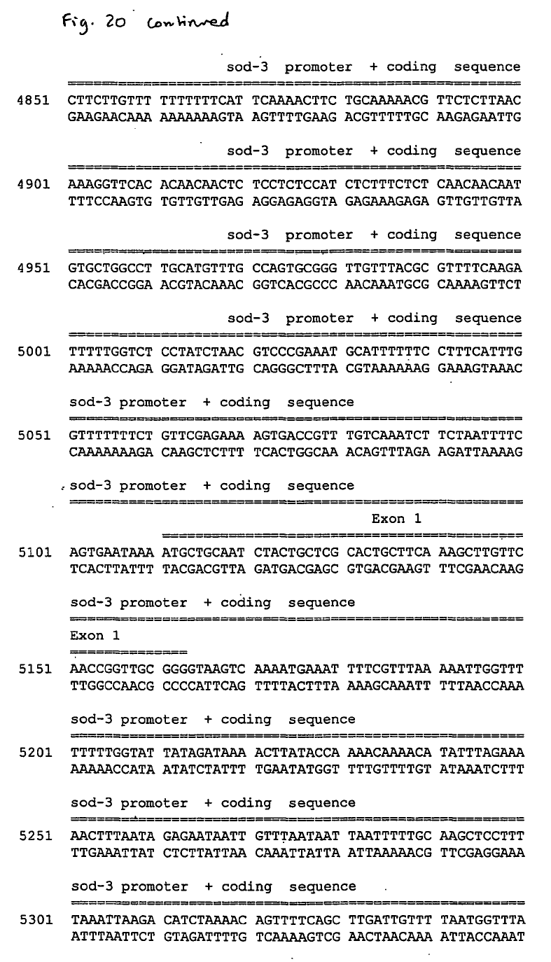

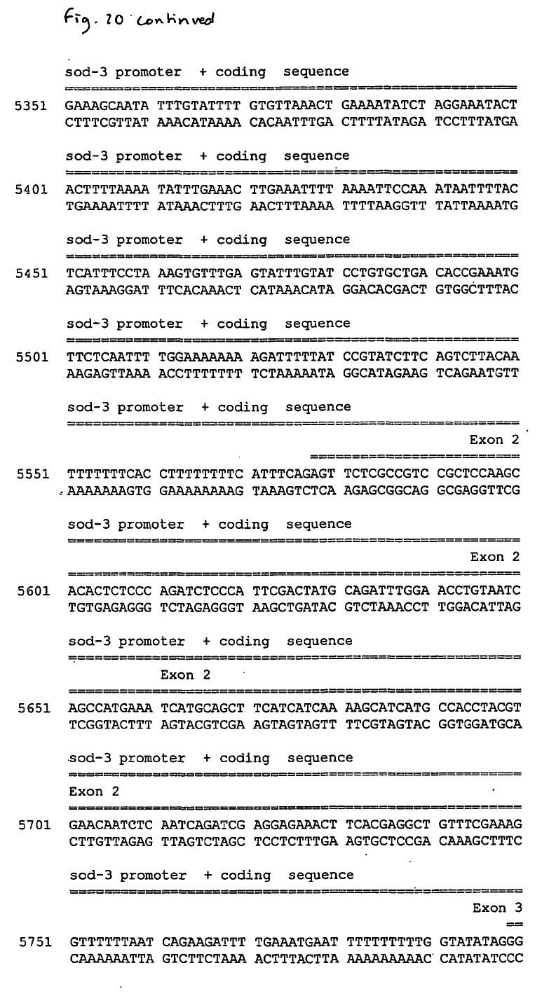

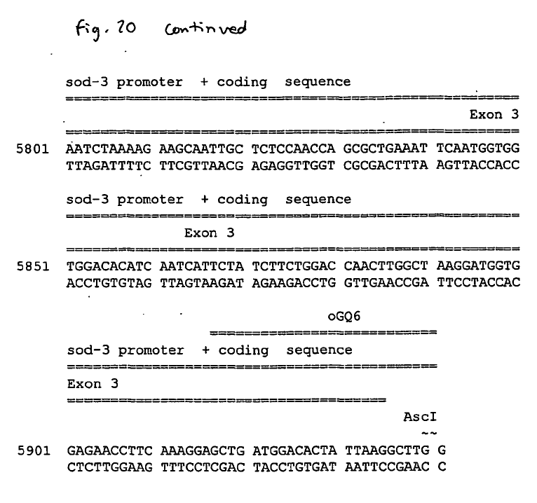

FIG. 20 shows the complete nucleotide sequence of pGQ3.

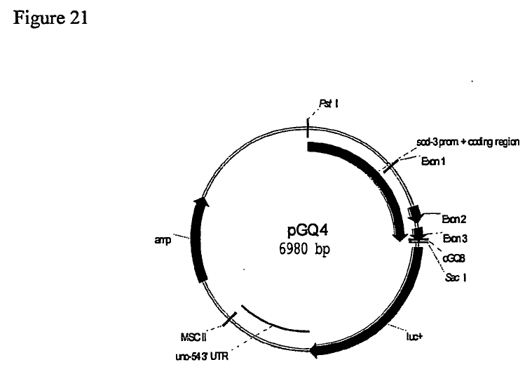

FIG. 21 is a representation of pGQ4.

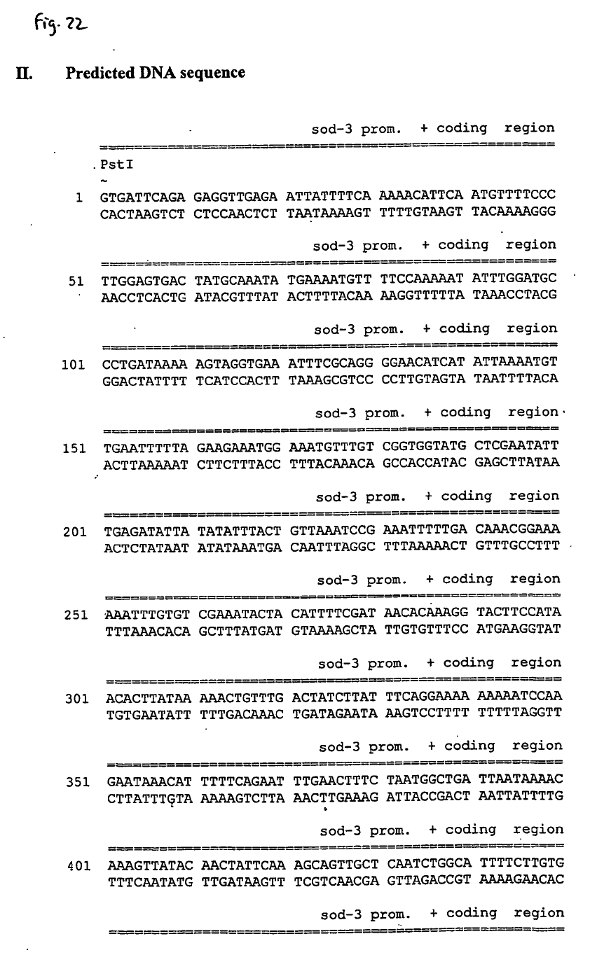

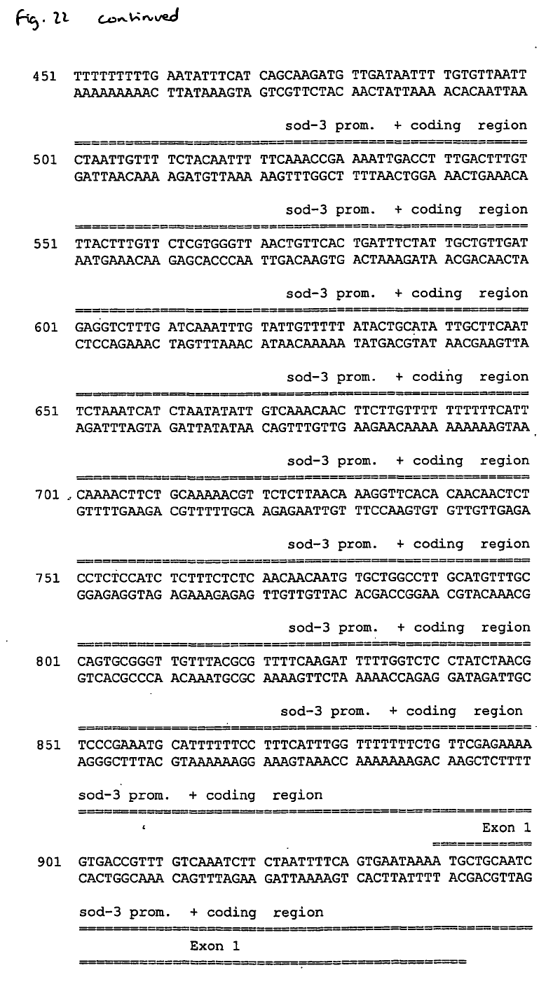

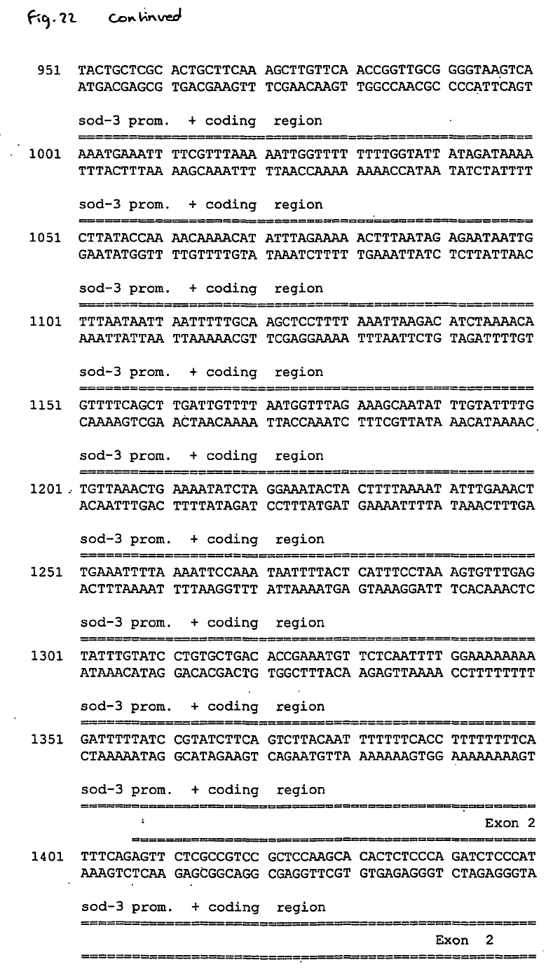

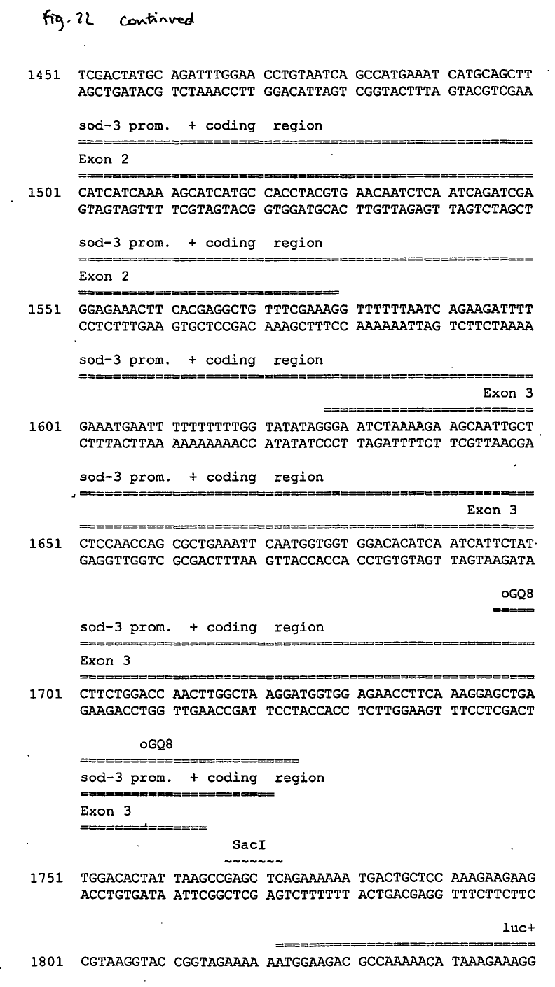

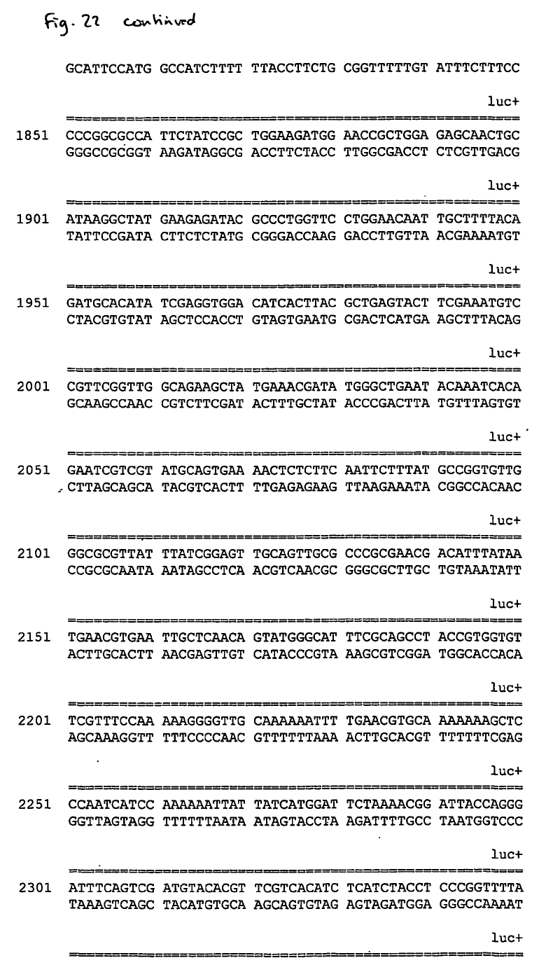

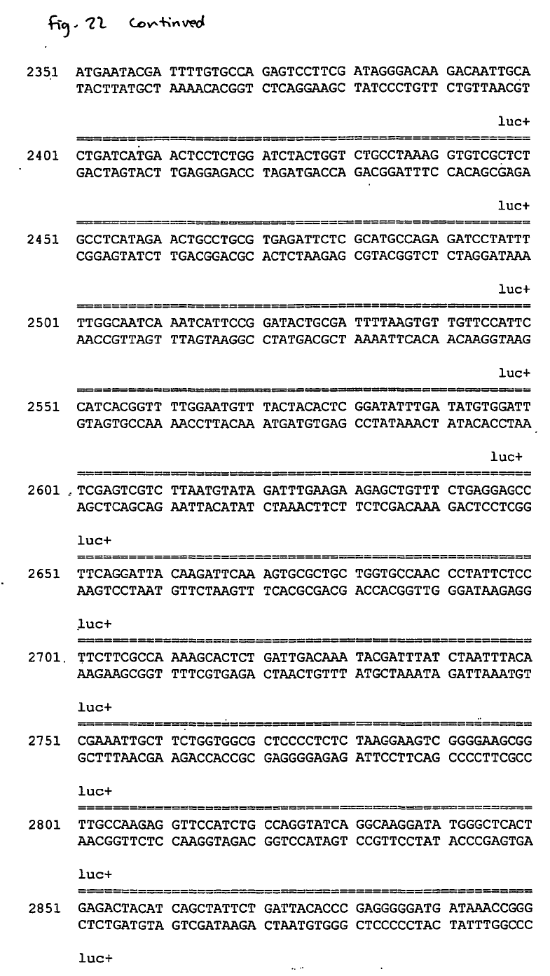

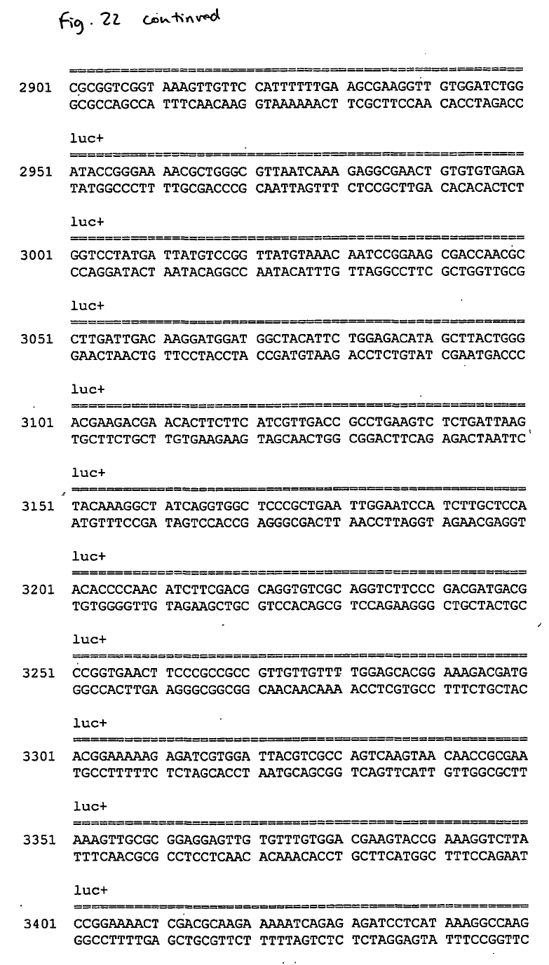

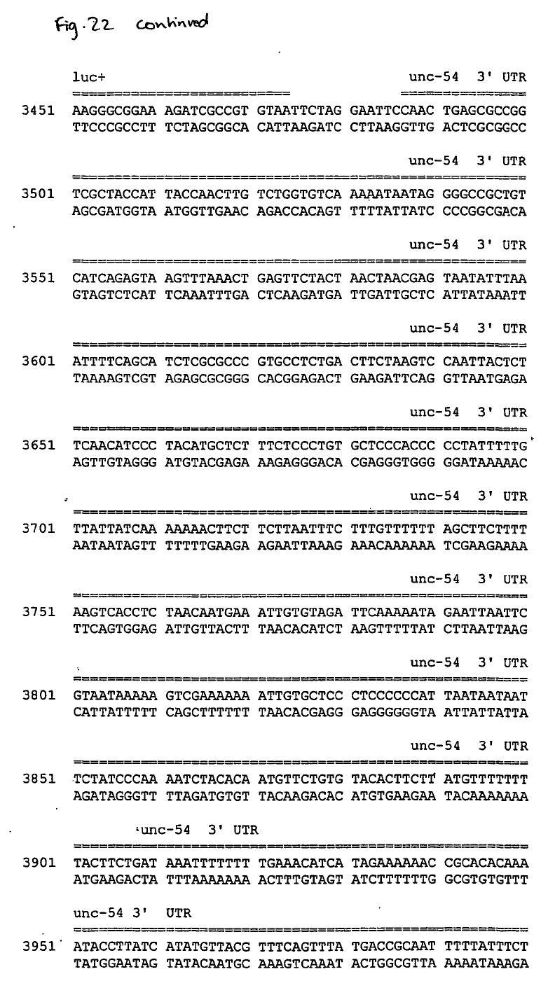

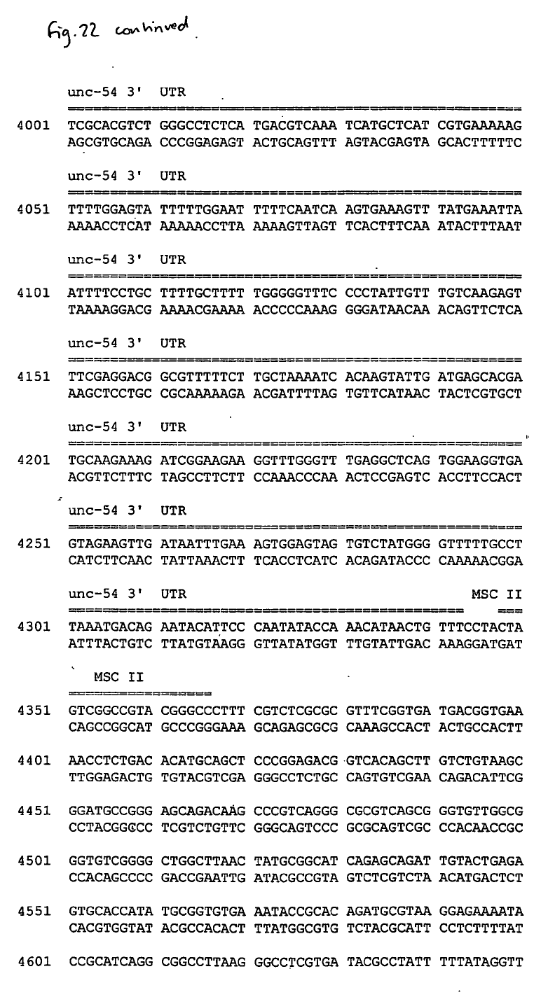

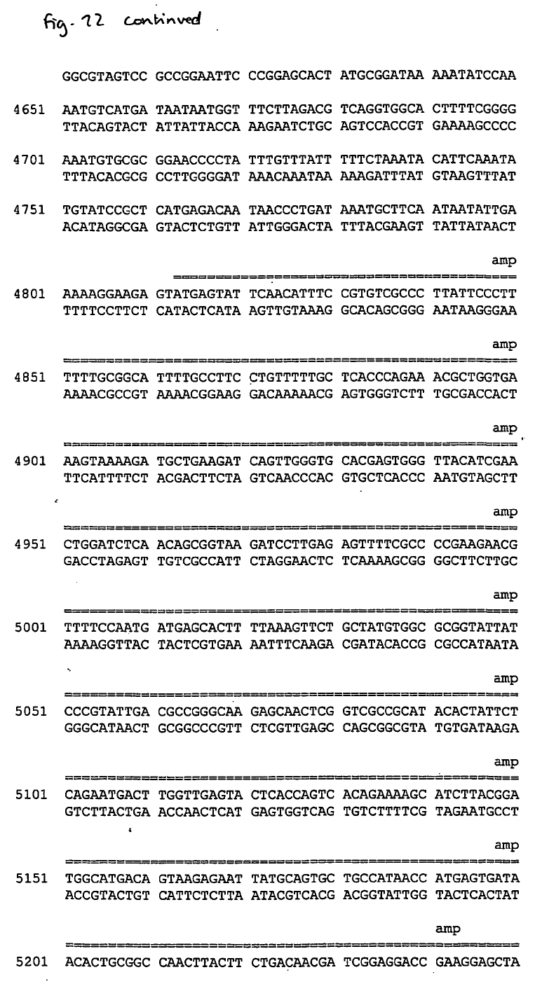

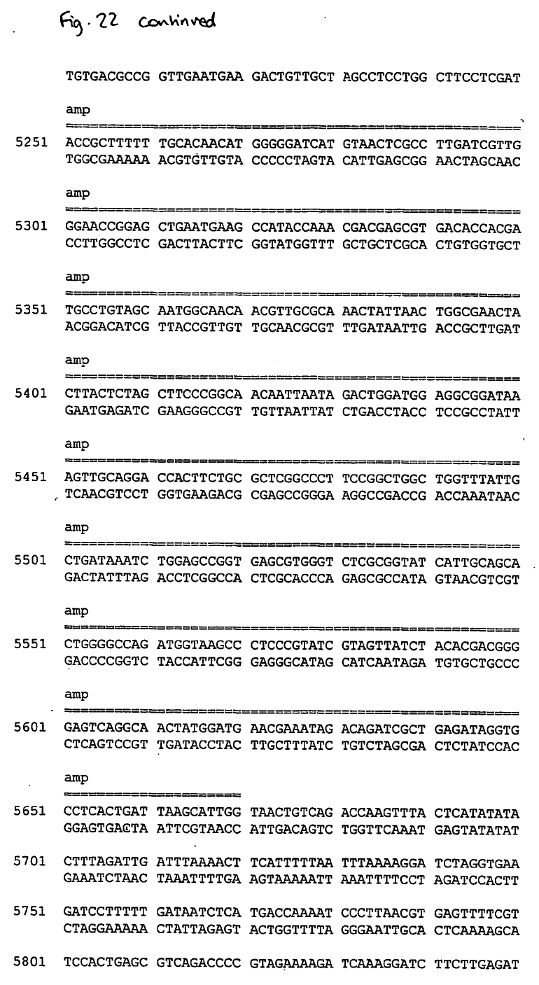

FIG. 22 shows the complete nucleotide sequence of pGQ4.

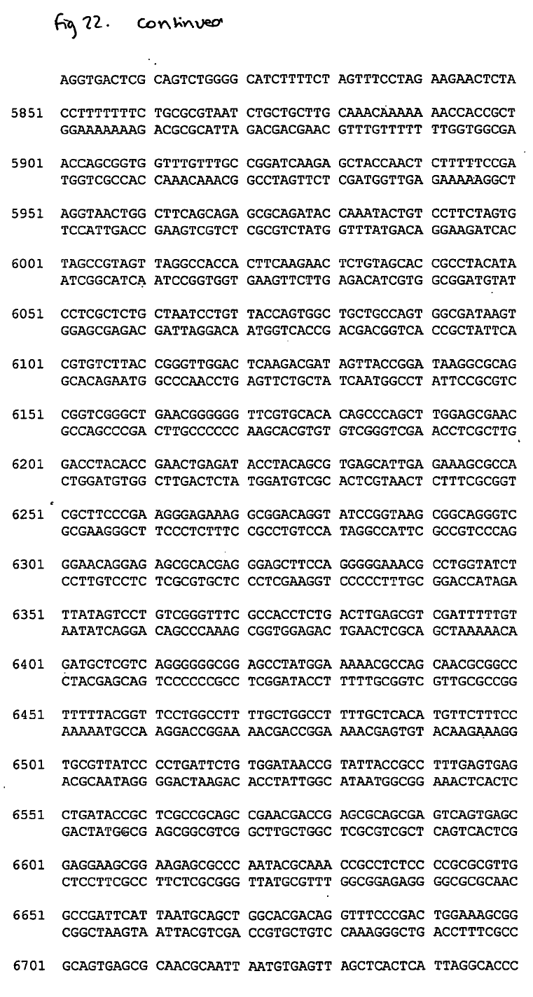

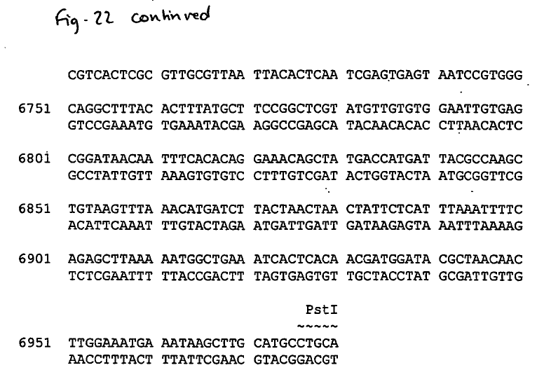

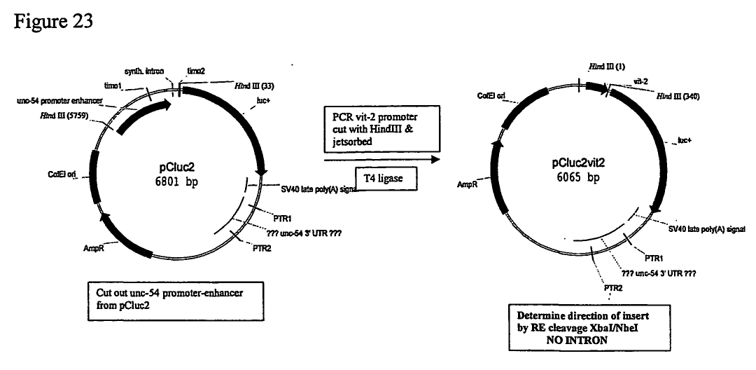

FIG. 23 illustrates the cloning of pCluc6.

### EXAMPLE 1

### Screening 23,040 Compounds for Activity in the Insulin-Receptor Pathway

Materials used

9 cm plates seeded with OP50,
three weeks old stock plates of daf-2(m41)
M9 buffer
S-complete buffer
96-well plates flat bottom NUCLON Surface
96-well plates U-bottom for dilutions compounds
HB101 bacteria (routinely available)
compounds (80 per 96-well plates) 10 mM concentration in 100% DMSO
   Method
   Test of the Batch of Bacteria to be Used as Food:
Growth of HB101
   fill a 2 liter Erlenmeyer sterile with 0.51 DYT medium
   inoculate with E-coli HB101 single colony
   let shake for 24 hours at 250 rpm and 37 C
   centrifuge in sterile 250 ml centrifuge tubes 10 min 10000 rpm.
   resuspend in 120 ml S-basal medium (pipette up and down and shake)
   transfer to 8 15 ml falcon tubes that were weighed in advance
   centrifuge second time 10 min 6000 rpm
   weigh the pellet
   store at 4 C
Test of the batch:
   chunk a couple of plates of m41
   bleach plates after 4 days, let eggs hatch on unseeded plate at 15 C
   wash off L1's after one night
   bring 50 L1 in 80 μl S-complete in one 96 well plate
   add 10 μl 2% DMSO
   add 10 μl of 1.25% of the batch of bacteria to be tested
   put plate in closed box in the 21 C incubator
   check on number of dauers after three days of growth, should be no more then 10
   if the batch is approved, it can be stored undiluted at 4 C for several weeks
      Protocol
      Thursday:
chunk 9 cm plates (take 1 plate/96-well plate to be filled)
grow in middle incubator at 15 C (preferably same shelf)
   Monday: Bleach Plates
wash off in M9
10 plates/falcon 15 ml
put washed off plates back in 15 C incubator (only uncontaminated ones)
spin down at 1300 rpm/3 min
suck off M9
add bleach
when most worms are broken, add sucrose, shake, add 2 ml M9
spin at 1300 rpm/3 min
carefully remove eggs from bottom of layer of M9, bring in new falcon
add M9 to 15 ml
spin down 1300 rpm/3 min
add M9
spin down 1300 rpm/3 min
suck away M9 to 1 ml
divide eggs from one falcon over 3 unseeded plates
put plates at 15 C to let eggs hatch
   Tuesday
   a) Preparation of the Compound-Plates
dilute aliquot of compound in 96-well plate to 200 μM in S-buffer (DMSO conc. 2%).
replicate plates: four plates 10 μl 200 μM compound per well
write number and replicate number on plates
if there was no DMSO in col 1 and 12 of the aliquoted plate it has to be added (add 11 μl of 2% DMSO)
write number of the plate and the replicate on the lid of the plates
   b) Preparation of the Worms Solution
1) “bleached L1's”
   wash L1 off plates in S-complete, 4 plates/15 ml falcon
   spin down at 1300 rpm/3 min
   add fresh S-complete to 100 ml
   count worms in 10 μl
   keep worm suspension at 15 C while counting
   dilute further to approximately 50 worms/80 μl, count again
   mix well
2) “washed L1's”
   wash off plates that were washed yesterday
   spin down (1300 rpm/3 min), add S-complete, wash twice
   filter suspension over 11 micron mesh over embroidery hoop into lid of 9 cm plate
   wash L1's one more time
   dilute to 50 worms/80 μl in the same way as bleached L1
      c) Final Steps:
   add 1.25% freshly diluted HB101 bacteria to worm suspension so that final concentration is 0.125% (1 volume of bacteria to 8 of worms)
   add 90 μl of worm-bacteria suspension/well with electronic pipette
   put plates in closed boxes with wet tissues in 21° C. incubator
   monitor temperature in control box in incubator while growing (try to put boxes at the same shelf, avoid contact of the boxes to metal of cooling device!)
      Friday: Scoring:
1. count 8 negative control wells/plate
2. plot the average and variance of the negative controls from each plate
3. check for differences between boxes, differently treated L1's and replicates
4. if necessary define several groups, remove outliers
5. make a distribution of the negative controls per group (plot # of wells to the number of worms/well)
6. for each defined group: fit a negative binomial distribution to the negative controls and determine the number of adults for a cut-off confidentiality of about 1% and about 0.1% (both sides for screen of dauer rescue and dauer enhancers)
7. screening for dauer rescue is possible if average of negative control is between 0 and 15 adults/well, screening for dauer enhancers is possible if the average is above 5
8. screen through the plates and count the wells with high number of adults
9. if the number of adults in the well is below the cut-off value leave it
10. if the number of adults is above or at the 1% cut-off value circle the well as positive (for each of the replicate with a different color) and write the number in the circle
11. if the number of adults is above the 0.1% cut-off value estimate the number of adults
12. Put the lids of the 4 replicates of the same plate on top of each other
13. Search for wells with 2 or more positives in the 4 (or 3) replicates
14. Write down the number of the adults of each of the 4 (or 3) replicates
   Robustness

While the controls active in the pathway show the sensitivity of the assay (see FIGS. 2 and 3), its specificity is determined by testing arrange of compounds outside the pathway. Together with the reference compounds acting in the insulin signalling pathway, of which only Wortmannin and vanadates were active, anti-diabetics with a mode of action outside the insulin pathway, including 3 guanidine derivatives (acting on glucose uptake and metabolism), 5 PPARγ ligands (stimulating adipocyte differentiation) and 6 sulphonylureas (which act by increasing insulin secretion) were tested. None was found to be active in the assay. Further confirmation of the specificity of the screen is derived from testing a library of 800 compounds from Tocris-Cookson, containing mainly neurological actives, at 20 μM in triplicates. Only 4 compounds rescued dauer formation, a rate not higher than for random libraries (see results).

TABLE 2

| | | | | | Concentrations |
|---|---|---|---|---|---|
| | | | | | tested in μM |
| | | | | | (lethal) |
| | | | | | rescue, |
| Name of compound | supply | MW | drug class/disease area/action(s) | solvent | dauer enhancer |
| Synthalin | ICN | 354.5 | guanidine derivative, also NMDA | DMSO | (333; 166.7; 83.3; |
| | | | antagonist | | 33.3); 20; 16.6; |
| | | | | | 8.3; 3.3 |
| Metformin HCl (1,1- | Sigma | 165.6 | guanidine derivative, biguanides, | DMSO | 333; 166.7; 83.3; |
| dimethylbiguanide) | | | MOA?: decrease hepatic glucose | | 33.3; 20 |
| | | | production | | |
| Phenformin HCl | Sigma | 241.7 | guanidine derivative, biguanides, | DMSO | 333; 166.7; 83.3; |
| (phenethylbiguanide) | | | MOA?: decrease hepatic glucose | | 33.3; 20 |
| | | | production | | |
| HNMPA(AM)3 | Calbiochem | 454.4 | insulin receptor tyrosine kinase inhibitor | DMSO | 20 |
| Rapamycin | ICN | 914.2 | insulin signalling enhancer, inhibitor of | DMSO | 33.3; 16.6; 8.3; |
| | | | the mammalian target of rapamycin | | |
| | | | (mTOR) which is a downstream target | | |
| | | | of Akt and implicated in Akt's negative | | |
| | | | regulation of insulin signalling i.e. | | |
| | | | serine/threonine phosphorylation of | | |
| | | | IRS-1 | | |
| Quercetin | Sigma | 338.3 | insulin signalling inhibitor, inhibitor of | DMSO | 20 |
| | | | phosphatidylinositol 3-kinase and of | | |
| | | | several other ATP-requiring enzymes | | |
| | | | e.g. PI4K, PKC, EGFR, calcium, | | |
| | | | SERCA activator by interacting with | | |
| | | | nucleotide binding site to mask PLB | | |
| | | | inhibition | | |
| okadaic acid | Calbiochem | 805 | insulin signalling inhibitor, inhibits PP2A | DMSO | 10; 5; 2.5; 0.6 |
| | | | and PP1 | | |
| PD 98059 | Calbiochem | 267.3 | insulin signalling inhibitor, MEK1 | DMSO | 20 |
| | | | inhibitor | | |
| Wortmannin | Sigma | 428.4 | insulin signalling inhibitor, | DMSO | 20 |
| | | | phosphatidylinositol 3-kinase | | |
| | | | inhibitor (potent and specific), | | |
| | | | inhibitor of neutrophil activation and | | |
| | | | of FMLP-mediated phospholipase D | | |
| | | | activation | | |
| LY 294002 | Sigma | 307.3 | insulin signalling inhibitor, | DMSO | 100, 20 |
| | | | phosphatidylinositol 3-kinase inhibitor | | |
| | | | (specific) | | |
| phorbol 12-myristate | Biomol | 616.8 | insulin signalling inhibitor, PKC activator | DMSO | 20 |
| 13-acetate (PMA) | | | (elicits serine/threonine phosphorylation | | |
| | | | of IRS-1) | | |
| Phosphatidylinositol- | Alexis | 1123.1 | insulin signalling, identical to | DMSO | 2.8; 1.4; 0.7 |
| 3,4,5-trisphosphate | | | endogenous PI(3,4,5)P3 (not an analog | | |
| [stearyl, arachidonoyl, | | | containing only saturated fatty acid | | |
| tetraammonium salt) | | | residues, therefore greater biological | | |
| | | | activity), activates Ca2+-insensitive | | |
| | | | PKC, activates Akt (a serine/threonine | | |
| | | | kinase) by directly interacting with the | | |
| | | | Akt pleckstrin homology (PH) domain | | |
| Phosphatidylinositol- | Calbiochem | 1056.2 | insulin signalling, mimics endogenous | DMSO | 3.17; 1.9; 1.58; |
| 3,4-bisphosphate [L- | | | PI(3,4)P2, activates Ca2+-insensitive | | 0.79 |
| alpha-] (dipalmitoyl, | | | PKC, activates Akt (a serine/threonine | | |
| pentaammonium salt) | | | kinase) by directly interacting with the | | |
| | | | Akt pleckstrin homology (PH) domain | | |
| Phosphatidylinositol- | Calbiochem | 1170.2 | insulin signalling, mimics endogenous | DMSO | 2.96; 1.74; 1.48 |
| 3,4,5-trisphosphate | | | PI(3,4,5)P3, activates Ca2+-insensitive | | |
| [L-alpha-] | | | PKC, activates Akt (a serine/threonine | | |
| (dipalmitoyl, | | | kinase) by directly interacting with the | | |
| heptaammonium salt) | | | Akt pleckstrin homology (PH) domain | | |
| Thalidomide | ICN | 258.2 | insulin signalling, TNF inhibitor | DMSO | 333; 166.7; 83.3; |
| | | | | | 33.3; 20 |
| Perhexiline | Sigma | 393.6 | insulin, carbohydrate metabolism, | DMSO | (333; 166.7; 83.3; |
| | | | inhibitor of myocardial carnitine | | 33.3); 20; 16.6; |
| | | | palmitoyltransferase-1 (“antidiabetics”), | | 8.3; 3.3 |
| | | | sodium, calcium, dual Na+/Ca2+ (T- | | |
| | | | type) channel blocker, anti-angina | | |
| | | | (coronary vasodilator), diuretic | | |
| L-arginine | Sigma | 174.2 | nitric oxide, insulin secretagogue (NO | water | 333; 166.7; 83.3; |
| | | | dependent) | | 33.3; 20 |
| D-arginine | Sigma | 174.2 | nitric oxide, negative control of L- | water | 20 |
| | | | arginine (insulin secretagogue) | | |
| LY 171883 | Sigma | 318.4 | PPARgamma activator (weak), | DMSO | 20 |
| | | | selective LTD4 antagonist | | |
| linoleic acid (9,12- | Sigma | 280.4 | PPARgamma ligand | DMSO | (333; 166.7; 83.3; |
| octadecadienoic acid) | | | | | 33.3); 20; 16.6; |
| | | | | | 8.3; 3.3 |
| Linolenic acid | Sigma | 278.4 | PPARgamma ligand | DMSO | (333; 166.7; 83.3; |
| (9,12,15- | | | | | 33.3); 20; 16.6; |
| octadecatrienoic acid) | | | | | 8.3; 3.3 |
| Eicosatetraynoic acid | ICN | 296.5 | PPARgamma ligand, insulin sensitizers, | DMSO | 333; 166.7; 83.3; |
| [5,8,11,14-] (ETYA) | | | eicosanoid | | 33.3; 20 |
| Rosiglitazone (BRL49653) | | 359 | PPARgamma-specific agonist (insulin- | water | 909; 500; 263; |
| | | | sensitizing properties, used in type II | | 135; 55; 27.6; |
| | | | diabetes) | | 13.85 |
| Chelerythrine chloride | Sigma | 383.8 | protein kinase C inhibitor (potent, | DMSO | 10 |
| | | | selective, IC50 0.7 μM) | | |
| Cantharidic acid | Sigma | 214.2 | protein phosphatase 2A inhibitor (IC50 | DMSO | 20 |
| | | | 53 nM) | | |
| Phenylarsine oxide | Calbiochem | 168 | PTP inhibitor, also inhibits PI3-kinase | DMSO | 20 |
| | | | activity | | |
| Bromotetramisole | Biomol | 373.2 | PTP inhibitor, also well known inhibitor | water | 20 |
| oxalate [L-p-] | | | of alkaline phosphatase, mimics the | | |
| | | | action of orthovanadate in the | | |
| | | | potentiation of fluorouracil | | |
| | | | antiproliferative activity | | |
| Bromotetramisole | Biomol | 373.2 | PTP inhibitor, also well known inhibitor | water | 20 |
| oxalate [D-p-] | | | of alkaline phosphatase, mimics the | | |
| | | | action of orthovanadate in the | | |
| | | | potentiation of fluorouracil | | |
| | | | antiproliferative activity: inactive isomer, | | |
| | | | negative control | | |
| Dephostatin | Calbiochem | 168.2 | PTP inhibitor, IC50 7.7 μM, also nitric | DMSO | 333; 166.7; 83.3; |
| | | | oxide donor (stable NO donor for S- | | 20 |
| | | | nitrosation of proteins) | | |
| vanadium(II) chloride | Aldrich- | 121.85 | PTP inhibitor, vanadium compound | DMSO | 20 |
| | Sigma | | | | |
| vanadium(III) chloride | Aldrich- | 157.3 | PTP inhibitor, vanadium compound | DMSO | 1000; 500; 250; |
| | Sigma | | | | 100; 20 |
| vanadium(III) oxide | Aldrich- | 149.88 | PTP inhibitor, vanadium compound | DMSO | 20 |
| | Sigma | | | | |
| vanadium(IV) oxide | Aldrich- | 165.88 | PTP inhibitor, vanadium compound | DMSO | 20 |
| | Sigma | | | | |
| vanadium(V) oxide | Aldrich- | 181.88 | PTP inhibitor, vanadium compound | DMSO | 20 |
| | Sigma | | | | |
| vanadyl sulfate | Aldrich- | 163 | PTP inhibitor, vanadium compound | DMSO | 1000; 500; 250; |
| | Sigma | | | | 100; 20 |
| vanadyl trifluoride | Fluka- | 123.94 | PTP inhibitor, vanadium compound | DMSO | 20 |
| | Sigma | | | | |
| mpV (Pic) (monoperxo | Calbiochem | 257.1 | PTP inhibitor, vanadium compound | DMSO | 1000; 500; |
| (picolinato) | | | | | 100; 20 |
| oxovanadate(V)) | | | | | |
| sodium | Sigma | 121.9 | PTP inhibitor, vanadium compound, | water | 1000; 500; 250; |
| metavanadate | | | also inhibits ATPase and alkaline | | 100; 20 |
| | | | phosphatase | | |
| sodium | Sigma | 183.9 | PTP inhibitor, vanadium compound, | water | 1000; 500; 250; |
| orthovanadate | | | also inhibits ATPase and alkaline | | 100; 20 |
| | | | phosphatase | | |
| bpV (Phen) | Calbiochem | 404.3 | PTP inhibitor, vanadium compound, | DMSO | 1000; 500; 250; |
| (Potassium | | | potent | | 100; 20 |
| Bisperoxo (1,10- | | | | | |
| phen anthroline) | | | | | |
| oxovanadate(V)) | | | | | |
| bpV(bipy) (potassium | Alexis | 326.2 | PTP inhibitor, vanadium compound, | DMSO | 1000; 500; 250; |
| bisperoxo(bipyridine) | | | potent | | 100; 20 |
| oxovanadate(V) | | | | | |
| bpV(Hopic) (dipotassium | Alexis | 347.2 | PTP inhibitor, vanadium compound, | DMSO | 1000; 500; 250; |
| bisperoxo | | | potent | | 100; 20 |
| (5-hydroxy | | | | | |
| pyridine-2- | | | | | |
| carboxyl)- | | | | | |
| oxovanadate(V) | | | | | |
| bpV(pic) | Alexis | 367.3 | PTP inhibitor, vanadium compound, | DMSO | 1000; 500; 250; |
| (dipotassium | | | potent | | 100; 20 |
| bisperoxo(picolinato) | | | | | |
| oxovanadate(V) | | | | | |
| acetohexamide | ICN | 324.4 | sulfonylureas, first generation, MOA: | DMSO | 333; 166.7; 83.3; |
| | | | insulin secretagogue by blocking | | 33.3; 20 |
| | | | K+(ATP) channels | | |
| chlorpropamide | Sigma | 276.7 | sulfonylureas, first generation, MOA: | DMSO | 333; 166.7; 83.3; |
| | | | insulin secretagogue by blocking | | 33.3; 20 |
| | | | K+(ATP) channels | | |
| tolazamide | Sigma | 311.4 | sulfonylureas, first generation, MOA: | DMSO | 333; 166.7; 83.3; |
| | | | insulin secretagogue by blocking | | 33.3; 20 |
| | | | K+(ATP) channels | | |
| tolbutamide | Sigma | 270.3 | sulfonylureas, first generation, MOA: | DMSO | 333; 166.7; 83.3; |
| | | | insulin secretagogue by blocking | | 33.3; 20 |
| | | | K+(ATP) channels | | |
| glipizide | RBI | 445.53 | sulfonylureas, second generation, | DMSO | 333; 166.7; 83.3; |
| | | | MOA: insulin secretagogue by blocking | | 33.3; 20 |
| | | | K+(ATP) channels | | |
| glyburide | Tocris | 494.1 | sulfonylureas, second generation, | DMSO | 333; 166.7; 83.3; |
| (glybenclamide) | | | MOA: insulin secretagogue by blocking | | 33.3; 20 |
| | | | K+(ATP) channels | | |
| diazoxide | Tocris | 230.7 | potassium, K+ channel opener, | DMSO | 333; 166.7; 83.3; |
| | | | avtivates ATP-sensitive K+ channels, | | 33.3; 20 |
| | | | antihypertensive, also stimulates K+ | | |
| | | | channels in pancreatic islet cells | | |
| | | | (prodiabetic side effects), diabetes | | |

Data Aquisition

All screening was done at 20 μM compound concentration in quadruplicates, except 2000 compounds of Diverset in triplicates. Confirmation was done at 4 concentrations. Questionable dose responses were repeated, if necessary at lower concentrations and/or 2 fold dilution steps. All worms that bypassed dauer stage, L4s and adults, were counted under a Leica MZ12 dissection scope and together referred to as number of adults per well. First, the 8 negative controls (column 1) of all plates were counted, typically between 800 and 1280 (25 to 40 plates times 4 per screening session). Data were transferred to Excel files and average and variance of the 8 controls of each plate calculated and plotted.

Outliers of unusual high average or variance were removed for calculation, since they were found to have an inappropriately large effect on the calculations below (3 plates in the example of FIG. 5a). Counting errors were found to have a rather weak effect. The number of wells was plotted against the number of adults per well and a negative binomial distribution fitted by maximum likelihood. In some cases it was necessary to split a session in two or three different subsessions mainly due to differences in incubator location or worm handling.

Then the number of adults per well where the cumulative negative binomial distribution was closest to 99% was determined and referred to as 1% cut-off. In the example shown in FIG. 6, 20 adults per well were at 1.10% indicating that the probability to have 20 or more adults per well is 1.10%. This calculates to a 4% chance for a single false positive in quadruplicates, but only to a 0.07% chance for a double false positive. Therefore a compound is positive, if at least 2 replicates have values at the cut-off or higher. In addition the 0.1% cut-off was determined similarly (24 adults in the example shown in FIG. 6) and if at least 2 replicates were reaching that stronger value the compound was referred to as strong positive.

The plates were then screened through quickly to find wells with a high number adults, which were counted and if found to reach the cut-off value the position on the lid was circled and the exact value written in the circle. For higher numbers above the 0.1% cut-off an estimate rather than an exact count proved sufficient. Finally the transparent lids of the 4 replicate plates were stacked on top of each other and by looking through them it was determined whether 2 or more lids were circled in any position. For those positions all the positive values were written into an excel file.

For confirmation by dose response fresh compound in 100% DMSO was used and from an initial dilution to 2% DMSO three further dilutions in 3.16 fold steps with a 2% DMSO solution in S-buffer were prepared. In that way 4 concentrations, 20 μM, 6.3 μM, 2 μM and 0.63 μM were tested, all in 0.2% DMSO background. Both columns 1 and 12 contained 0.2% DMSO as control. Each plate contained 20 different compounds, with 4 replica-plates of them.

TABLE 3

| | | comp1 | comp2 | comp3 | Comp4 | comp5 | comp6 | comp7 | comp8 | comp9 | comp1 | |
|---|---|---|---|---|---|---|---|---|---|---|---|---|
| | 1 | 2 | 3 | 4 | 5 | 6 | 7 | 8 | 9 | 10 | 11 | 12 |
| A | cntrl | 20 μM | 20 μM | 20 μM | 20 μM | 20 μM | 20 μM | 20 μM | 20 μM | 20 μM | 20 μM | cntrl |
| B | cntrl | 6 μM | 6 μM | 6 μM | 6 μM | 6 μM | 6 μM | 6 μM | 6 μM | 6 μM | 6 μM | cntrl |
| C | cntrl | 2 μM | 2 μM | 2 μM | 2 μM | 2 μM | 2 μM | 2 μM | 2 μM | 2 μM | 2 μM | cntrl |
| D | cntrl | 0.6 μM | 0.6 μM | 0.6 μM | 0.6 μM | 0.6 μM | 0.6 μM | 0.6 μM | 0.6 μM | 0.6 μM | 0.6 μM | cntrl |
| E | cntrl | 20 μM | 20 μM | 20 μM | 20 μM | 20 μM | 20 μM | 20 μM | 20 μM | 20 μM | 20 μM | cntrl |
| F | cntrl | 6 μM | 6 μM | 6 μM | 6 μM | 6 μM | 6 μM | 6 μM | 6 μM | 6 μM | 6 μM | cntrl |
| G | cntrl | 2 μM | 2 μM | 2 μM | 2 μM | 2 μM | 2 μM | 2 μM | 2 μM | 2 μM | 2 μM | cntrl |
| H | cntrl | 0.6 μM | 0.6 μM | 0.6 μM | 0.6 μM | 0.6 μM | 0.6 μM | 0.6 μM | 0.6 μM | 0.6 μM | 0.6 μM | cntrl |
| | | comp1 | comp1 | comp1 | Comp1 | comp1 | comp1 | comp1 | comp1 | comp1 | comp2 | |
| | | 1 | 2 | 3 | 4 | 5 | 6 | 7 | 8 | 9 | 0 | |

“Cntrl”—abbreviation for control

For some compounds an additional dose response with 7 concentrations was made, mostly with 2 fold dilutions to obtain 20 μM, 10 μM, 5 μM, 2.5 μM, 1.25 μM, 0.63 μM and 0.31 μM. In that case also row H contained controls. Each plate contained 10 different compounds, with 4 replica-plates of them. An example of the 26 negative controls of 16 plates showes the variability of the mean while the standard deviation remained fairly constant (FIG. 5b). Furthermore, the negative controls expressed as percentage of the plate mean were approximately normal distributed (FIG. 7). Therefore all data were normalized according to the calculation below which centers value of no effect at 0 and calibrates the y-axis to standard deviations. The concentrations are on the x-axis in logarithmic scale. All 4 replicates are plotted, in addition a smoothed line through the averages is plotted.
value in SD=(number of adults of the well−1)/SD of the controls of the set average controls of the plate

A compound was determined as confirmed and designated a hit when either the average or two of the 4 values reached 2.5 SD (corresponds to 99.3% confidence) at any concentration and a reasonable dose-response is apparent.

Results

From 23.040 compounds a total of 300 positives were obtained during the screening, of which 173 could be reconfirmed.

TABLE 4

| | | | confirmed | % re- | |
|---|---|---|---|---|---|
| library name | size | Positives | hits | confirmed | hit rate |
| Library 1 | 2000 | 33 | 3 | 9% | 0.15% |
| Library 2 | 5040 | 92 | 62 | 67% | 1.23% |
| Library 3 | 16000 | 175 | 108 | 62% | 0.68% |
| TOTAL | 23040 | 300 | 173 | 57% | 0.75% |

To estimate the potency of the screen, that is to estimate what fraction of compounds that could have been identified with the assay have actually been identified during the screen, an analysis on 47 compounds defining 11 chemical clusters has been performed: 36 of these compounds have been confirmed. Another 40 compounds, which were not found to be active in the original screen but are members of those clusters, were submitted to dose response confirmation. 4 more hits have been identified. In total 40 compounds could be confirmed, 36 of the screen positives and 4 from the extra set. Hence 90% of the final hits of these clusters were detected in the original screen and 10% were missed.

TABLE 5

| | | confirmed | similar | extra | final |
|---|---|---|---|---|---|
| Cluster | positives | hits | negatives | hits | hits |
| 1 | 5 | 4 | 1 | 0 | 4 |
| 3 | 6 | 6 | 7 | 1 | 7 |
| 4 | 7 | 6 | 1 | 0 | 6 |
| 5 | 4 | 4 | 1 | 0 | 4 |
| 6 | 3 | 3 | 5 | 1 | 4 |
| 7 | 5 | 3 | 1 | 0 | 3 |
| 8 | 3 | 1 | 7 | 1 | 2 |
| 9 | 5 | 4 | 13 | 0 | 4 |
| 12 | 5 | 2 | 1 | 0 | 2 |
| 13 | 2 | 2 | 2 | 0 | 2 |
| 15 | 2 | 1 | 1 | 1 | 2 |
| Total | 47 | 36 | 40 | 4 | 40 |

Conclusions
1. A mutation in the C. elegans insulin receptor, daf-2(m41), was used successfully in an pharmacological assay for compounds acting in the downstream pathway.
2. The assay is sensitive enough to screen at 20 μM compound concentrations, at which there were nearly no problems due to lethality (27 of 23,040).
3. A hit rate of 0.75% from combinatorial chemistry libraries has been obtained, strongly dependent on the library.
4. The screen is specific for the insulin receptor pathway and is unlikely to yield many hits upstream e.g. stimulating insulin release.
5. The active compounds are candidates to cure insulin resistance and therefore of potential therapeutic use in type II diabetes and obesity.
6. Since the compounds bypass the need of insulin they are also of potential use in type I diabetes.
7. The major mode of compound entry in C. elegans is the gut which pre-selects for orally active compounds.

8. The activity is retrieved from a whole-organism readout leaving intact tissue-specific insulin signalling and feedback loops.

TABLE 6
Retest of 94 compounds at 20 μM on 3 different
daf-2 alleles, m41 at 211C, e1368 and e1370 at 251C.
Values: 3: all replicates above 99% threshold, 2:
median above 99.9% threshold, 1: median above 99%
threshold, 0: median below 99% threshold.

| ID | MW | Plate | Row | Col | m41 | e1368 | e1370 |
|---|---|---|---|---|---|---|---|
| 217485 | 547.18 | 1 | A | 2 | 1 | 1 | 0 |
| 211706 | 472.55 | 1 | A | 3 | 3 | 3 | 0 |
| 181141 | 459.51 | 1 | A | 4 | 3 | 1 | 0 |
| 259910 | 384.53 | 1 | A | 5 | 0 | 0 | 0 |
| 194326 | 393.49 | 1 | A | 6 | 2 | 0 | 0 |
| 217336 | 420.04 | 1 | A | 7 | 3 | 3 | 0 |
| 267546 | 372.51 | 1 | A | 8 | 0 | 0 | 0 |
| 228433 | 405.56 | 1 | A | 9 | 0 | 0 | 0 |
| 264792 | 436.94 | 1 | A | 10 | 3 | 0 | 0 |
| 255126 | 431.50 | 1 | A | 11 | 3 | 0 | 0 |
| 100718 | 399.88 | 1 | B | 2 | 3 | 0 | 0 |
| 182576 | 486.39 | 1 | B | 3 | 0 | 0 | 0 |
| 232839 | 475.30 | 1 | B | 4 | 3 | 1 | 0 |
| 217339 | 394.00 | 1 | B | 5 | 3 | 1 | 0 |
| 217341 | 394.00 | 1 | B | 6 | 3 | 2 | 0 |
| 118776 | 437.52 | 1 | B | 7 | 2 | 0 | 0 |
| 118783 | 452.35 | 1 | B | 8 | 3 | 2 | 0 |
| 118789 | 442.35 | 1 | B | 9 | 2 | 1 | 0 |
| 248144 | 440.89 | 1 | B | 10 | 3 | 0 | 0 |
| 234291 | 462.76 | 1 | B | 11 | 0 | 0 | 0 |
| 212465 | 367.39 | 1 | C | 2 | 0 | 0 | 0 |
| 144331 | 363.98 | 1 | C | 3 | 0 | 0 | 0 |
| 138263 | 372.51 | 1 | C | 4 | 2 | 1 | 0 |
| 264982 | 352.48 | 1 | C | 5 | 1 | 1 | 0 |
| 267659 | 386.93 | 1 | C | 6 | 1 | 0 | 0 |
| 115771 | 391.50 | 1 | C | 7 | 3 | 0 | 0 |
| 105359 | 326.40 | 1 | C | 8 | 3 | 0 | 0 |
| 267467 | 419.37 | 1 | C | 9 | 0 | 0 | 0 |
| 236867 | 480.25 | 1 | C | 10 | 0 | 0 | 0 |
| 225671 | 365.44 | 1 | C | 11 | 0 | 0 | 0 |
| 225858 | 444.33 | 1 | D | 2 | 0 | 1 | 0 |
| 225615 | 523.23 | 1 | D | 3 | 0 | 1 | 0 |
| 101025 | 431.42 | 1 | D | 4 | 1 | 0 | 0 |
| 255192 | 420.38 | 1 | D | 5 | 3 | 1 | 0 |
| 217850 | 391.27 | 1 | D | 6 | 3 | 0 | 0 |
| 214475 | 329.36 | 1 | D | 7 | 3 | 1 | 0 |
| 114446 | 479.71 | 1 | D | 8 | 2 | 0 | 0 |
| 261736 | 378.40 | 1 | D | 9 | 2 | 0 | 0 |
| 210145 | 373.84 | 1 | D | 10 | 0 | 0 | 0 |
| 114816 | 304.40 | 1 | D | 11 | 2 | 0 | 0 |
| 210877 | 445.34 | 1 | E | 2 | 0 | 0 | 0 |
| 189119 | 379.38 | 1 | E | 3 | 3 | 1 | 0 |
| 203845 | 379.38 | 1 | E | 4 | 1 | 0 | 0 |
| 190303 | 303.36 | 1 | E | 5 | 0 | 0 | 0 |
| 253121 | 524.23 | 1 | E | 6 | 3 | 1 | 0 |
| 228525 | 462.45 | 1 | E | 7 | 2 | 1 | 0 |
| 118761 | 381.89 | 1 | E | 8 | 2 | 0 | 0 |
| 228489 | 428.55 | 1 | E | 9 | 1 | 0 | 0 |
| 250480 | 332.36 | 1 | E | 10 | 2 | 1 | 0 |
| 118765 | 416.33 | 1 | E | 11 | 3 | 0 | 0 |
| 254230 | 425.24 | 1 | F | 2 | 0 | 0 | 0 |
| 255339 | 427.69 | 1 | F | 3 | 2 | 1 | 0 |
| 250001 | 383.24 | 1 | F | 4 | 2 | 0 | 0 |
| 255335 | 383.24 | 1 | F | 5 | 2 | 2 | 0 |
| 263986 | 330.86 | 1 | F | 6 | 0 | 0 | 0 |
| 236861 | 486.21 | 1 | F | 7 | 0 | 0 | 0 |
| 104926 | 280.35 | 1 | F | 8 | 0 | 1 | 0 |
| 133891 | 272.30 | 1 | F | 9 | 0 | 0 | 0 |
| 154290 | 364.27 | 1 | F | 10 | 2 | 0 | 0 |
| 189005 | 363.76 | 1 | F | 11 | 1 | 0 | 0 |
| 195094 | 346.29 | 1 | G | 2 | 2 | 0 | 0 |
| 203897 | 408.21 | 1 | G | 3 | 3 | 0 | 0 |
| 210775 | 510.21 | 1 | G | 4 | 1 | 0 | 0 |
| 214387 | 376.64 | 1 | G | 5 | 3 | 0 | 0 |
| 219414 | 318.33 | 1 | G | 6 | 1 | 0 | 0 |
| 228301 | 311.36 | 1 | G | 7 | 0 | 0 | 0 |
| 228488 | 414.53 | 1 | G | 8 | 1 | 0 | 0 |
| 230672 | 376.21 | 1 | G | 9 | 0 | 0 | 0 |
| 231561 | 365.88 | 1 | G | 10 | 0 | 0 | 0 |
| 236341 | 386.41 | 1 | G | 11 | 0 | 0 | 0 |
| 249726 | 422.19 | 1 | H | 2 | 1 | 0 | 0 |
| 249746 | 373.33 | 1 | H | 3 | 2 | 0 | 0 |
| 253051 | 311.57 | 1 | H | 4 | 0 | 0 | 0 |
| 257516 | 380.73 | 1 | H | 5 | 0 | 0 | 0 |
| 258687 | 305.36 | 1 | H | 6 | 0 | 0 | 0 |
| 260067 | 357.18 | 1 | H | 7 | 0 | 0 | 0 |
| 265080 | 346.29 | 1 | H | 8 | 0 | 1 | 0 |
| 268434 | 372.42 | 1 | H | 9 | 0 | 0 | 0 |
| 273546 | 443.05 | 1 | H | 10 | 0 | 0 | 0 |
| 276545 | 337.70 | 1 | H | 11 | 1 | 0 | 0 |
| 278617 | 430.05 | 2 | A | 2 | 0 | 0 | 0 |
| 279528 | 316.34 | 2 | A | 3 | 0 | 0 | 0 |
| 281078 | 344.25 | 2 | A | 4 | 3 | 0 | 0 |
| 283400 | 390.31 | 2 | A | 5 | 0 | 0 | 0 |
| 284204 | 301.26 | 2 | A | 6 | 0 | 0 | 0 |
| 284316 | 385.22 | 2 | A | 7 | 0 | 0 | 0 |
| 286676 | 354.15 | 2 | A | 8 | 0 | 0 | 0 |
| 301158 | 475.86 | 2 | A | 9 | 3 | 2 | 0 |
| 304896 | 432.26 | 2 | A | 10 | 0 | 0 | 0 |
| 307069 | 362.82 | 2 | A | 11 | 0 | 0 | 0 |
| 309471 | 453.32 | 2 | B | 2 | 0 | 0 | 0 |
| 310513 | 318.13 | 2 | B | 3 | 2 | 1 | 0 |
| 313944 | 416.29 | 2 | B | 4 | 0 | 0 | 0 |
| 316982 | 516.85 | 2 | B | 5 | 2 | 0 | 0 |

| | | | |
|---|---|---|---|
| number or compounds active | 53 | 25 | 0 |
| percentage of compounds active | 56% | 27% | 0% |

### EXAMPLE 2

### Automatic Data Aquisition with Nile Red staining

Material:

Hardware:

microtiterplates: 96 well black U-shaped plates (DYNEX Microfluor7 2)
Wallac 1420 plate reader (Victor 2):
Nile Red protocol:
   excitation=530 nm
   emission=590 nm
Counting time: 1 second
CW lamp energy: 30445
Emission aperture: damp
Counter position: top
Measurement height: 3 mm from bottom of the plate
   Consumables:
Nile Red (Sigma, N-3013).
Ivermectin (ICN, 196009)
   Method:
Prepare a 100 mM solution of Nile Red (Nile Blue A Oxazone) in pure methanol. Centrifugate to remove the saturated solution from the undissolved Nile Red.
Dilute in steps of 10 with buffer to 500 μM.
Add 1:1 Nile Red to the worms and incubate for 30 min at room temperature.
Add 10 μM ivermectin final concentration and incubate for 30 min at room temperature.
Measure.

### EXAMPLE 3

### Automatic Data Aquisition with a Vit-2::Luciferase Reporter

Material:

Hardware:

microtiterplates: 96 well white U-shaped plates (DYNEX Microfluor â 2)
Wallac 1420 plate reader (Victor 2):
Luciferase protocol
Emission-Filter: no filter
Counting time: 3 seconds
Emission aperture: normal
   Consumables:
Triton X-100 (BDH, 306324N)
Dual-Luciferaseâ Reporter Assay System (Promega, E4550)
   Method:
Add Triton X-100 (1% final concentration) to lyse the worms.
Shake for 1 minute and freeze.
Thaw the plates and add 1:1 luciferine.
Shake for 1 minute and measure.

### EXAMPLE 4

### Construction of ctl-1::Luciferase and sod-3::Luciferase Reporters

1) Construction of pGQ1

1.1 PCR

PCR (turbo pfu) on N2 genomic DNA with:

oGQ1:ctl-1::GFP fw (PstI):

5′ AAAACTGCAGCCAATGCATTGGAAGAGATATTTTGCGCGTCAAATAT
GTTTTGTGTCC3′
oGQ2bis:ctl-1::GFP rv (BamHI)
5′ CGCGGATCCGGCCGATTCTCCAGCGACCG3′

1.2 Cloning
Digest of the PCR fragment with PstI and BamHI
Ligation into pDW2020 and transformation into DH10B
   2) Construction of pGQ2
   2.1 PCR

PCR (turbo pfu) on N2 genomic DNA with:

oGQ3:ctl-1::luciferase fw (StuI):

5′ CCAGGCCTGAGATATTTTGCGCGTCAAATATGTTTTGTGTCC3′
oGQ4:ctl-1::luciferase rv (SacI)
5′ CGGAGCTCCGATTGGATGTGGTGAGCAGG3′

2.2 Cloning
Digest of the PCR fragment with StuI and SacI
Ligation into pCluc6 and transformation into DH10B
   3) Construction of pGQ3
   3.1 PCR

PCR (turbo pfu) on N2 genomic DNA with:

oGQ7:sod-3 fw:

5′GCAGAATTTGCAAAACGAGCAGGAAAGTC3′
oGQ6:sod-3::luciferase rv (AscI)
5′TTGGCGCGCCAAGCCTTAATAGTGTCCATCAGC3′

3.2 Cloning
Digest of the PCR fragment with PstI and AscI
Ligation into pDW2020 and transformation into HD10B
   4) Construction of pGQ4
   4.1 PCR

PCR (turbo pfu) on N2 genomic DNA with:

oGQ7:sod-3 fw:

5′GCAGAATTTGCAAAACGAGCAGGAAAGTC3′
oGQ8:sod-3::luciferase rv (SacI)
5′CTGAGCTCGGCTTAATAGTGTCCATCAGC3′

4.2 Cloning
Digest of the PCR fragment with PstI and SacII
Ligation into pCluc6 and transformation into HD10B

### EXAMPLE 5

### Construction of pCluc6

Vector:

Restriction digest of pCluc2 with HindIII
Purification, protocol: Jetsorb
   Insert:

PCR the vit-2 promoter (248 bp in front of exon1 just before ATG) with primers (designed from ACeDB C42D8.2) that contain HindIII RE sites out of N2 genomic DNA:

vit-2F:
5′CCCCCAAGCTTCCATGTGCTAGCTGAGTTTCATCATGTCC3′
vit-2R:
5′CCCCCCAAGCTTGGCTGAACCGTGATTGG3′

Restriction digest on PCR product with HindIII
Purification, protocol: Jetsorb
   pCluc6:
T4 DNA ligation of vector and insert
Transformation into DH10B
Mini DNA preparation, protocol: Wizard SV Miniprep
determine direction of insert by RE cleavage XbaI/NheI
Maxi DNA preparation, protocol: Jetstar
Check maxiprep by sequencing with o-PUCI primer.
   Standard Methods and Worm Strains

Standard methods for culturing nematodes are described in Methods in Cell biology Vol. 48, 1995, ed. by Epstein and Shakes, Academic press. Standard methods are known for creating mutant worms with mutations in selected C. elegans genes, for example see J. Sutton and J. Hodgkin in “The Nematode Caenorhabditis elegans”, Ed. by William B. Wood and the Community of C. elegans Researchers CSHL, 1988 594-595; Zwaal et al, “Target-Selected Gene Inactivation in Caenorhabditis elegans by using a Frozen Transposon Insertion Mutant Bank” 1993, Proc. Natl. Acad. Sci. USA 90 pp 7431-7435; Fire et al, Potent and Specific Genetic Interference by Double-Stranded RNA in C. elegans 1998, Nature 391, 860-811. A population of worms can be subjected to random mutagenesis by using EMS, TMP-UV or radiation (Methods in Cell Biology, Vol 48, ibid). Several selection rounds of PCR could then be performed to select a mutant worm with a deletion in a desired gene.

A range of specific C. elegans mutants are available from the C. elegans mutant collection at the C. elegans Genetic Center, University of Minnesota, St Paul, Minn.

E. coli strain OP50 can be obtained from the C. elegans Genetics Center, University of Minnesota, St Paul, Minn., USA.

1. A method for the identification of a compound which is capable of modulating insulin signalling pathways, which method comprises:
providing C. elegans dauer larvae;
contacting said larvae with a test compound; and
screening for release from the dauer larval state, wherein the C. elegans dauer larvae possess a sensitized genetic background, as compared to the reference daf-2 mutant e1370.

2. Method according to claim 1, in which the dauer larvae belong to a nematode strain which has an Insulin Sensitivity Value (“ISV”) that is greater than the ISV for the reference nematode strain CB1370, in particular more than 1% greater, preferably more than 5% greater, more preferably more than 10% greater, even more preferably more than 20% greater.

3. Method according to claim 1 and/or 2, in which the dauer larvae belong to a nematode strain which has an ISV that is >30%, preferably >40%, even more preferably >50%.

4. A method as claimed in claim 1 wherein the C. elegans dauer larvae are daf-2(m41) mutants.

5. A method as claimed in claim 1 wherein the C. elegans dauer larvae comprise a daf-2 class I allele other than daf-2(m41).

6. A method as claimed in claim 1 wherein the C. elegans dauer larvae comprise at least one loss-of-function or reduction-of-function mutation in a gene(s) downstream of the insulin receptor in the insulin signalling pathway.

7. A method as claimed in claim 6 wherein the C. elegans dauer larvae comprise a loss-of-function or reduction-of-function mutation in the age-1 gene.

8. A method as claimed in claim 6 wherein the C. elegans dauer larvae comprise loss-of-function or reduction-of-function mutations in the akt-1 gene and the akt-2 gene.

9. A method as claimed in claim 6 wherein the C. elegans dauer larvae comprise a loss-of-function or reduction-of-function mutation in the pdk-1 gene.

10. A method as claimed in claim 9 wherein the C. elegans dauer larvae are pdk-1(sa680) mutants.

11. A method as claimed in claim 1 wherein the C. elegans dauer larvae are larvae wherein the dauer phenotype is induced by treatment with an inhibitor inhibitor of at least one component of the insulin receptor signalling pathway.

12. A method as claimed in claim 11 wherein the inhibitor compound is an inhibitor of the C. elegans PI3-kinase.

13. A method as claimed in claim 12 wherein the inhibitor compound is wortmannin or LY294002.

14. A method as claimed in claim 1 wherein expression of at least one gene downstream of the insulin receptor in the insulin receptor signalling pathway in said C. elegans dauer larvae is inhibited by RNAi inhibition.

15. A method as claimed in claim 1 wherein the C. elegans dauer larvae comprise a gain-of-function mutation in the daf-16 gene.

16. A method as claimed in claim 1 wherein the C. elegans dauer larvae comprise a gain-of-function mutation in the daf-18 gene.

17. A method as claimed in claim 1 wherein the C. elegans dauer larvae comprise a gain-of-function mutation in the C. elegans homologue of the SHIP2 gene.

18. A method as claimed in claim 1 wherein the C. elegans larvae dauer comprise a gain-of-function mutation in the C. elegans homologue of the PTP-1B gene.

19. A method as claimed in claim 1 wherein the C. elegans dauer larvae exhibit a defect in perception of environmental signals.

20. A method as claimed in claim 19 wherein the said C. elegans dauer larvae comprise a mutation in the tph-1 gene.

21. A method as claimed in claim 20 wherein the said C. elegans dauer larvae are tph-1(mg280) mutants.

22. A method as claimed in claim 1 wherein the C. elegans dauer larvae comprise a daf-c mutation in a daf gene selected from the group consisting of daf-1, daf-4, daf-7, daf-8, daf-11, daf-14, daf-21, daf-19 and daf-28.

23. A method as claimed in claim 1 wherein the C. elegans dauer larvae comprise a mutation in a gene encoding a neuronal G-protein.

24. A method as claimed in claim 1 wherein the c. elegans dauer larvae are unc-64(e264); unc-31 (e928) mutants.

25. A method as claimed in any one of claims 1 to 24 wherein the step of screening for release from the dauer larval state comprises screening for adult C. elegans.

26. A method as claimed in any one of claims 1 to 24 wherein the step of screening for release from the dauer larval state comprises screening for changes in fat storage.

27. A method as claimed in any one of claims 1 to 24 wherein said C. elegans dauer larvae further comprise a reporter transgene comprising a promoter which is capable of directing strong gene expression in adult C. elegans and no or weak expression in dauer larvae or vice versa operably linked to a reporter gene and the step of screening for release from the dauer larval state comprises screening for changes in expression of the said reporter gene.

28. A method for the identification of a compound which is capable of modulating insulin signalling pathways, which method comprises:
providing C. elegans dauer larvae;
contacting said larvae with a test compound; and
screening for release from the dauer larval state, wherein conditions of the assay are selected such that a basal level of release from the dauer larval state is observed in the absence of the test compound.

29. A method as claimed in claim 28 wherein the basal level of release from the dauer larval state is between 0.1% and 40%.

30. A method as claimed in claim 29 wherein the basal level of release from the dauer larval state is between 1% and 30%.

31. A method as claimed in claim 30 wherein the basal level of release from the dauer larval state is between 2% and 20%.

32. A method as claimed in any one of claims 28 to 31 wherein the C. elegans dauer larvae are daf-2(m41) mutants.

33. A method as claimed in any one of claims 28 to 31 wherein the C. elegans dauer larvae are daf-2; daf-18 double mutants.

34. A method as claimed in any one of claims 28 to 31 wherein the C. elegans dauer larvae are Daf-d mutants.

35. A method as claimed in any one of claims 28 to 31 wherein the C. elegans dauer larvae comprise a gain-of-function mutation in the pdk-1 gene.

36. A method as claimed in claim 35 wherein the C. elegans dauer larvae are pdk-1(mg142) mutants.

37. A method as claimed in any one of claims 28 to 31 wherein the C. elegans dauer larvae comprise a gain-of-function mutation in the akt-1 gene.

38. A method as claimed in claim 37 wherein the C. elegans dauer larvae are akt-1(mg144) mutants.

39. A method as claimed in any one of claims 28 to 31 wherein the C. elegans dauer larvae are daf-16; daf-2 double mutants and further comprise a transgene capable of expressing a mammalian homolog of the daf-16 protein.

40. A method as claimed in claim 39 wherein the mammalian homolog of the daf-16 protein is the human FKHR protein, the human FKHRL1 protein or the human AFX protein.

41. A method as claimed in claim 28 wherein said C. elegans dauer larvae are larvae which have been treated with pheromone to reduce that fraction of worms growing to adults to below 40%.

42. A method as claimed in claim 41 wherein said C. elegans dauer larvae are larvae which have been treated with pheromone to reduce that fraction of worms growing to adults to below 30%.

43. A method as claimed in claim 42 wherein said C. elegans dauer larvae are larvae which have been treated with pheromone to reduce that fraction of worms growing to adults to below 20%.

44. A method as claimed in any one of claims 28 to 43 wherein the step of screening for release from the dauer larval state comprises screening for adult C. elegans.

45. A method as claimed in any one of claims 28 to 43 wherein said C. elegans larvae further comprise a reporter transgene comprising a promoter which is capable of directing strong gene expression in adult C. elegans and no or weak expression in dauer larvae or vice versa operably linked to a reporter gene and the step of screening for rescue of the daf-2 mutation comprises screening for expression of the said reporter gene.

46. A method as claimed in any one of claims 28 to 43 wherein the step of screening for release from the dauer larval state comprises screening for changes in fat storage.

47. A method for the identification of a compound which is capable of modulating insulin signalling pathways, which method comprises:
a) providing a sample of nematode worms (preferably eggs, L1 or L2 worms, and most preferably L1 worms);
b) keeping said sample under conditions such, without the presence of any compound(s) to be tested, at least 50%, and preferably at least 60%, and more preferably at least 70%, even more preferably at least 80%, such as 85-100% of the nematodes present in said sample would enter the dauer state (at least during the time used for the assay);
c) exposing the sample to the compound(s) to be tested;
d) measuring either the number of worms that enter the dauer state, and/or measuring the number of worms that grow into adults.

48. Method according to claim 47, in which the conditions used in step b) are such that, in the presence of a reference compound at a suitable concentration, the amount of worms that enter the dauer state is at least 10% less, preferably at least 20% less, more preferably at least 30% less, than the amount of worms that would enter the dauer state without the presence of any such reference compound (at least during the time used for the assay).

49. Method according to claim 46 and/or 47, in which the conditions used in step b) are such that, in the presence of a reference compound at a suitable concentration, the amount of worms that enter the dauer state is less than 50%, preferably less than 40%, even more preferably less than 30% (at least during the time used for the assay).

50. Method according to any of claims 47-49, in which the nematode worms that form the sample belong to a nematode strain that has an Insulin Sensitivity Value (“ISV”) that is greater than the ISV for the reference nematode strain CB1370, in particular more than 1% greater, preferably more than 5% greater, more preferably more than 10% greater, even more preferably more than 20% greater.

51. Method according to any of claims 47-50, in which the nematode worms that form the sample belong to a nematode strain which has an ISV that is >30%, preferably >40%, even more preferably >50%.

52. Method according to any of claims 47-50, in which the nematodes used in the sample are daf-2(m41) mutants.

53. Use of at least one nematode worm, which has an increased sensitivity of the insulin signalling pathway, in an assay for the identification of a compound which is capable of modulating insulin signalling pathways.

54. Use according to claim 53, in which the nematode worm belongs to a strain that has an Insulin Sensitivity Value (“ISV”) that is greater than the ISV for the reference nematode strain CB1370, in particular more than 1% greater, preferably more than 5% greater, more preferably more than 10% greater, even more preferably more than 20% greater.

55. Use according to claim 53 and/or 54, in which the nematode worm belongs to a strain that has an Insulin Sensitivity Value (“ISV”) that is >30%, preferably >40%, even more preferably >50%

56. Use according to any of claims 53-55, in which the nematode worm used is a daf-2(m41) mutant.

57. Use according to any of claims 53-56, in an assay that is carried out in a multi-well plate format.

58. Use according to any of claims 53-57, in an assay that is carried out in an automated fashion.

59. Use according to any of claims 53-58, in an assay based on the dauer phenotype as a biological read out, such as on the entry into, the bypass of and/or the rescue from the dauer state, and/or on any other property which results from and/or is associated with the so-called dauer decision.

60. Use according to claim 59, in an assay based on entry into the dauer state and/or bypass of the dauer state as a biological read out.

61. Use according to claim 59, in an assay based on rescue from the dauer state as a biological read out.

62. Use according to any of claims 53-61, for the identification of a small molecule and/or a small, peptide.


SEQUENCE LISTING (15 sequences)

SEQ ID NO: 1 - DNA, 58 bp, Artificial Sequence
  Oligonucleotide oGQ1ctl-1::GFP fw (PstI)
aaaactgcagccaatgcattggaagagatattttgcgcgtcaaatatgttttgtgtcc

SEQ ID NO: 2 - DNA, 29 bp, Artificial Sequence
  Oligonucleotide oGQ2bisctl-1::GFP rv (BamHI)
cgcggatccggccgattctccagcgaccg

SEQ ID NO: 3 - DNA, 42 bp, Artificial Sequence
  Oligonucleotide oGQ3ctl-1::luciferase fw (StuI)
ccaggcctgagatattttgcgcgtcaaatatgttttgtgtcc

SEQ ID NO: 4 - DNA, 29 bp, Artificial Sequence
  Oligonucleotide oGQ4ctl-1::luciferase rv (SacI)
cggagctccgattggatgtggtgagcagg

SEQ ID NO: 5 - DNA, 29 bp, Artificial Sequence
  Oligonucleotide oGQ7sod-3 fw
gcagaatttgcaaaacgagcaggaaagtc

SEQ ID NO: 6 - DNA, 33 bp, Artificial Sequence
  Oligonucleotide oGQ6sod-3::luciferase rv (AscI)
ttggcgcgccaagccttaatagtgtccatcagc

SEQ ID NO: 7 - DNA, 29 bp, Artificial Sequence
  Oligonucleotide oGQ8sod-3::luciferase rv (SacI)
ctgagctcggcttaatagtgtccatcagc

SEQ ID NO: 8 - DNA, 40 bp, Artificial Sequence
  Oligonucleotide vit-2F
cccccaagcttccatgtgctagctgagtttcatcatgtcc

SEQ ID NO: 9 - DNA, 29 bp, Artificial Sequence
  Oligonucleotide vit-2R
ccccccaagcttggctgaaccgtgattgg

SEQ ID NO: 10 - DNA, 4292 bp, Artificial Sequence
  Plasmid pDW2020
atgaccatgattacgccaagcttgcatgcctgcaggtcgactctagaggatccccgggattggccaaaggacccaaaggtatgtttcgaatgatactaacataacatagaacattttcaggaggacccttggctagcgtcgacggtaccatggggcgcgccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatgggtaagtttaaacatatatatactaactaaccctgattatttaaattttcagccaacacttgtcactactttctgttatggtgttcaatgcttctcgagatacccagatcatatgaaacggcatgactttttcaagagtgccatgcccgaaggttatgtacaggaaagaactatatttttcaaagatgacgggaactacaagacacgtaagtttaaacagttcggtactaactaaccatacatatttaaattttcaggtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttgtaagtttaaacttggacttactaactaacggattatatttaaattttcagaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagggccggccgagctccgcatcggccgctgtcatcagatcgccatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagct

SEQ ID NO: 11 - DNA, 5442 bp, Artificial Sequence
  Plasmid pGQ1
atgaccatgattacgccaagcttgcatgcctgcagccaatgcattggaagagatattttgcgcgtcaaatatgttttgtgtccccgtaatatttttttaaatcaaatttcacattttaaccataaaaaactctttcaaaagtgtaattttctacgcaaaaatgccgttcggatgaaaaattacttttgaaaaacaaactcgaaactacggtacgcaaaaaagtacatcggtgtttgcacataagtgaaaacaatgttgtttttttgtaattaaaatcgattaattttttttcccggaaaacaaaaacgttttcagcgtggatttctattgtttcttgcgtaaaaaaaaattatttaccaattttaaacgataatttccacgaattttcgccattaatctctcgattttgttgattcttgactccgagcaatctctccggttttcgcaaacgattatattatttatttgttttccttttcagtgccgattctcggaaattcaacagtaaatcttcaaaatgccaatgcttccccacatggtcaatctaagtgagtttctttgttacaaaatacacgtgatgtcagattgtctcatttcggtttgatctacgtagatctacaaaaaatgcgggaattgagccgcagagttctcaactgctttcgcatggttaagaacgtgcggacgtcaaattgttttgggcaaaaattcccgcattttttgtagatcaaaccgtaatgggacagtctggcaccacgtgactatatatttttagcggtcaacgacacaaaacccggaccaatggctgaggatcagctgaaagcttatagagatagaaatcaggtgagaaaaatcaatttcagcgattttcttcgcaatttatataaaaactgatttttccaggaaccccacctgctcaccacatccaatggagctccgatctactcgaagaccgccgtgctcaccgccggacgacgtggtccaatgctaatgcaggacatcgtttatatggacgagatggctcatttcgatcgtgaacgcatcccggagcgtgtcgtccatgccaaaggtggtggtgctcatggatacttcgaggtcacccatgacatcaccaagtactgtaaggccgatatgttcaacaaggtcggaaaacagacaccacttctcgttcgtttttcaacggtcgctggagaatcggccggatccccgggattggccaaaggacccaaaggtatgtttcgaatgatactaacataacatagaacattttcaggaggacccttggctagcgtcgacggtaccatggggcgcgccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatgggtaagtttaaacatatatatactaactaaccctgattatttaaattttcagccaacacttgtcactactttctgttatggtgttcaatgcttctcgagatacccagatcatatgaaacggcatgactttttcaagagtgccatgcccgaaggttatgtacaggaaagaactatatttttcaaagatgacgggaactacaagacacgtaagtttaaacagttcggtactaactaaccatacatatttaaattttcaggtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttgtaagtttaaacttggacttactaactaacggattatatttaaattttcagaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagggccggccgagctccgcatcggccgctgtcatcagatcgccatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagct

SEQ ID NO: 12 - DNA, 5686 bp, Artificial Sequence
  Plasmid pCluc6
atgactgctccaaagaagaagcgtaaggtaccggtagaaaaaatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctaggaattccaactgagcgccggtcgctaccattaccaacttgtctggtgtcaaaaataataggggccgctgtcatcagagtaagtttaaactgagttctactaactaacgagtaatatttaaattttcagcatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgtaagtttaaacatgatcttactaactaactattctcatttaaattttcagagcttaaaaatggctgaaatcactcacaacgatggatacgctaacaacttggaaatgaaataagcttgcatgcctgcaggccttggtcgactctagaggatcaaactgtattacttgaaacaatttagttatatgtttagaacccctcattcaaaattaatagacagggctctcaccgaatgttgcaatttgtttctgataagggtcacaaagcggagcgaatgcttgaatgtgtccatcaatgagcttatcaatgcgctaaaacgctataacttccatatgaagtcaatcgaacatatgtcaatctttagccgtatataaaggtgcactgaaaacagtccaatcacggttcagccatgaggtcgatccccggccgggattggccaaaggacccaaaggtatgtttcgaatgatactaacataacatagaacattttcaggaggacccttggagggtaccggggattggccaaaggacccaaaggtatgtttcgaatgatactaacataacatagaacattttcaggaggacccttgcttggagggtaccgagctcagaaaaa

SEQ ID NO: 13 - DNA, 6099 bp, Artificial Sequence
  Plasmid pGQ2
atgactgctccaaagaagaagcgtaaggtaccggtagaaaaaatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctaggaattccaactgagcgccggtcgctaccattaccaacttgtctggtgtcaaaaataataggggccgctgtcatcagagtaagtttaaactgagttctactaactaacgagtaatatttaaattttcagcatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgtaagtttaaacatgatcttactaactaactattctcatttaaattttcagagcttaaaaatggctgaaatcactcacaacgatggatacgctaacaacttggaaatgaaataagcttgcatgcctgcaggcctgagatattttgcgcgtcaaatatgttttgtgtccccgtaatatttttttaaatcaaatttcacattttaaccataaaaaactctttcaaaagtgtaattttctacgcaaaaatgccgttcggatgaaaaattacttttgaaaaacaaactcgaaactacggtacgcaaaaaagtacatcggtgtttgcacataagtgaaaacaatgttgtttttttgtaattaaaatcgattaattttttttcccggaaaacaaaaacgttttcagcgtggatttctattgtttcttgcgtaaaaaaaaattatttaccaattttaaacgataatttccacgaattttcgccattaatctctcgattttgttgattcttgactccgagcaatctctccggttttcgcaaacgattatattatttatttgttttccttttcagtgccgattctcggaaattcaacagtaaatcttcaaaatgccaatgcttccccacatggtcaatctaagtgagtttctttgttacaaaatacacgtgatgtcagattgtctcatttcggtttgatctacgtagatctacaaaaaatgcgggaattgagccgcagagttctcaactgctttcgcatggttaagaacgtgcggacgtcaaattgttttgggcaaaaattcccgcattttttgtagatcaaaccgtaatgggacagtctggcaccacgtgactatatatttttagcggtcaacgacacaaaacccggaccaatggctgaggatcagctgaaagcttatagagatagaaatcaggtgagaaaaatcaatttcagcgattttcttcgcaatttatataaaaactgatttttccaggaaccccacctgctcaccacatccaatcggagctcagaaaaa

SEQ ID NO: 14 - DNA, 5941 bp, Artificial Sequence
  Plasmid pGQ3
cgcgccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatgggtaagtttaaacatatatatactaactaaccctgattatttaaattttcagccaacacttgtcactactttctgttatggtgttcaatgcttctcgagatacccagatcatatgaaacggcatgactttttcaagagtgccatgcccgaaggttatgtacaggaaagaactatatttttcaaagatgacgggaactacaagacacgtaagtttaaacagttcggtactaactaaccatacatatttaaattttcaggtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttgtaagtttaaacttggacttactaactaacggattatatttaaattttcagaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagggccggccgagctccgcatcggccgctgtcatcagatcgccatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagtgattcagagaggttgagaattattttcaaaaacattcaatgttttcccttggagtgactatgcaaatatgaaaatgttttccaaaaatatttggatgccctgataaaaagtaggtgaaatttcgcaggggaacatcatattaaaatgttgaatttttagaagaaatggaaatgtttgtcggtggtatgctcgaatatttgagatattatatatttactgttaaatccgaaatttttgacaaacggaaaaaatttgtgtcgaaatactacattttcgataacacaaaggtacttccataacacttataaaaactgtttgactatcttatttcaggaaaaaaaaatccaagaataaacatttttcagaatttgaactttctaatggctgattaataaaacaaagttatacaactattcaaagcagttgctcaatctggcattttcttgtgtttttttttgaatatttcatcagcaagatgttgataattttgtgttaattctaattgttttctacaatttttcaaaccgaaaattgacctttgactttgtttactttgttctcgtgggttaactgttcactgatttctattgctgttgatgaggtctttgatcaaatttgtattgtttttatactgcatattgcttcaattctaaatcatctaatatattgtcaaacaacttcttgttttttttttcattcaaaacttctgcaaaaacgttctcttaacaaaggttcacacaacaactctcctctccatctctttctctcaacaacaatgtgctggccttgcatgtttgccagtgcgggttgtttacgcgttttcaagatttttggtctcctatctaacgtcccgaaatgcattttttcctttcatttggtttttttctgttcgagaaaagtgaccgtttgtcaaatcttctaattttcagtgaataaaatgctgcaatctactgctcgcactgcttcaaagcttgttcaaccggttgcggggtaagtcaaaatgaaattttcgtttaaaaattggttttttttggtattatagataaaacttataccaaaacaaaacatatttagaaaaactttaatagagaataattgtttaataattaatttttgcaagctccttttaaattaagacatctaaaacagttttcagcttgattgttttaatggtttagaaagcaatatttgtattttgtgttaaactgaaaatatctaggaaatactacttttaaaatatttgaaacttgaaattttaaaattccaaataattttactcatttcctaaagtgtttgagtatttgtatcctgtgctgacaccgaaatgttctcaattttggaaaaaaaagatttttatccgtatcttcagtcttacaatttttttcaccttttttttcatttcagagttctcgccgtccgctccaagcacactctcccagatctcccattcgactatgcagatttggaacctgtaatcagccatgaaatcatgcagcttcatcatcaaaagcatcatgccacctacgtgaacaatctcaatcagatcgaggagaaacttcacgaggctgtttcgaaaggttttttaatcagaagattttgaaatgaattttttttttggtatatagggaatctaaaagaagcaattgctctccaaccagcgctgaaattcaatggtggtggacacatcaatcattctatcttctggaccaacttggctaaggatggtggagaaccttcaaaggagctgatggacactattaaggcttgg

SEQ ID NO: 15 - DNA, 6980 bp, Artificial Sequence
  Plasmid pGQ4
gtgattcagagaggttgagaattattttcaaaaacattcaatgttttcccttggagtgactatgcaaatatgaaaatgttttccaaaaatatttggatgccctgataaaaagtaggtgaaatttcgcaggggaacatcatattaaaatgttgaatttttagaagaaatggaaatgtttgtcggtggtatgctcgaatatttgagatattatatatttactgttaaatccgaaatttttgacaaacggaaaaaatttgtgtcgaaatactacattttcgataacacaaaggtacttccataacacttataaaaactgtttgactatcttatttcaggaaaaaaaaatccaagaataaacatttttcagaatttgaactttctaatggctgattaataaaacaaagttatacaactattcaaagcagttgctcaatctggcattttcttgtgtttttttttgaatatttcatcagcaagatgttgataattttgtgttaattctaattgttttctacaatttttcaaaccgaaaattgacctttgactttgtttactttgttctcgtgggttaactgttcactgatttctattgctgttgatgaggtctttgatcaaatttgtattgtttttatactgcatattgcttcaattctaaatcatctaatatattgtcaaacaacttcttgttttttttttcattcaaaacttctgcaaaaacgttctcttaacaaaggttcacacaacaactctcctctccatctctttctctcaacaacaatgtgctggccttgcatgtttgccagtgcgggttgtttacgcgttttcaagatttttggtctcctatctaacgtcccgaaatgcattttttcctttcatttggtttttttctgttcgagaaaagtgaccgtttgtcaaatcttctaattttcagtgaataaaatgctgcaatctactgctcgcactgcttcaaagcttgttcaaccggttgcggggtaagtcaaaatgaaattttcgtttaaaaattggttttttttggtattatagataaaacttataccaaaacaaaacatatttagaaaaactttaatagagaataattgtttaataattaatttttgcaagctccttttaaattaagacatctaaaacagttttcagcttgattgttttaatggtttagaaagcaatatttgtattttgtgttaaactgaaaatatctaggaaatactacttttaaaatatttgaaacttgaaattttaaaattccaaataattttactcatttcctaaagtgtttgagtatttgtatcctgtgctgacaccgaaatgttctcaattttggaaaaaaaagatttttatccgtatcttcagtcttacaatttttttcaccttttttttcatttcagagttctcgccgtccgctccaagcacactctcccagatctcccattcgactatgcagatttggaacctgtaatcagccatgaaatcatgcagcttcatcatcaaaagcatcatgccacctacgtgaacaatctcaatcagatcgaggagaaacttcacgaggctgtttcgaaaggttttttaatcagaagattttgaaatgaattttttttttggtatatagggaatctaaaagaagcaattgctctccaaccagcgctgaaattcaatggtggtggacacatcaatcattctatcttctggaccaacttggctaaggatggtggagaaccttcaaaggagctgatggacactattaagccgagctcagaaaaaatgactgctccaaagaagaagcgtaaggtaccggtagaaaaaatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctaggaattccaactgagcgccggtcgctaccattaccaacttgtctggtgtcaaaaataataggggccgctgtcatcagagtaagtttaaactgagttctactaactaacgagtaatatttaaattttcagcatctcgcgcccgtgcctctgacttctaagtccaattactcttcaacatccctacatgctctttctccctgtgctcccaccccctatttttgttattatcaaaaaaacttcttcttaatttctttgttttttagcttcttttaagtcacctctaacaatgaaattgtgtagattcaaaaatagaattaattcgtaataaaaagtcgaaaaaaattgtgctccctccccccattaataataattctatcccaaaatctacacaatgttctgtgtacacttcttatgttttttttacttctgataaattttttttgaaacatcatagaaaaaaccgcacacaaaataccttatcatatgttacgtttcagtttatgaccgcaatttttatttcttcgcacgtctgggcctctcatgacgtcaaatcatgctcatcgtgaaaaagttttggagtatttttggaatttttcaatcaagtgaaagtttatgaaattaattttcctgcttttgctttttgggggtttcccctattgtttgtcaagagtttcgaggacggcgtttttcttgctaaaatcacaagtattgatgagcacgatgcaagaaagatcggaagaaggtttgggtttgaggctcagtggaaggtgagtagaagttgataatttgaaagtggagtagtgtctatggggtttttgccttaaatgacagaatacattcccaatataccaaacataactgtttcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgtaagtttaaacatgatcttactaactaactattctcatttaaattttcagagcttaaaaatggctgaaatcactcacaacgatggatacgctaacaacttggaaatgaaataagcttgcatgcctgca